SEQUENCE LISTING (11 sequences)

SEQ ID NO: 1 - DNA, 4107 bp, Synthetic construct
  source 1..4107: mol_type = other DNA
atggacaagaagtactctatcggcctggacatcggcaccaacagcgtgggctgggccgtcatcaccgacgagtacaaggtgccttctaagaagttcaaggtgctgggcaacaccgaccgccattctatcaagaagaacctgatcggcgccctgctgttcgactctggcgagaccgccgaggccaccagactgaagcggaccgcccgacgccgatacaccagacggaagaacagaatctgctaccttcaggagatcttcagcaacgagatggccaaggtggacgactctttcttccatcgcctggaggagagcttcctggtggaggaggacaagaagcatgagcgccatcctatcttcggcaacatcgtggacgaggtggcctaccatgagaagtaccctaccatctaccatctgaggaagaagctggtggactctacggacaaggccgacctgagacttatctacctggccctggcccatatgatcaagttccggggccatttcctcatcgagggcgacctcaaccccgacaacagcgacgtggacaagctgttcatccagttggtgcagacctacaaccagcttttcgaggagaaccccatcaacgcctctggcgtggacgccaaggccatcctgagtgcccgcctgtctaagagccgcagacttgagaacctgatcgcccagctgcccggcgagaagaagaacggcctgttcggcaaccttatcgccctgtctctgggccttacccctaacttcaagtctaacttcgacctggccgaggacgccaagctgcagcttagcaaggacacctacgacgacgacttggacaacctgcttgcccagatcggcgaccagtacgccgacctgttcctggccgccaagaacttgagcgacgccatcctgcttagcgacatcctgagagtcaacaccgagatcaccaaggcccctctgtctgccagcatgatcaagcggtacgacgagcatcaccaggacctgaccctgttgaaggccctcgtgcgacagcagctgcctgagaagtacaaggagatcttctttgaccagagcaagaacggctacgccggctacatcgacggcggcgcctctcaggaggagttctacaagttcatcaagcccatcctggagaagatggacggcaccgaggagcttctggtcaagctgaacagggaggacctgcttaggaagcagcgcaccttcgacaacggctcaatccctcatcagatccacctgggcgagttgcatgccatcctcagacgccaggaggacttctaccccttcctgaaggacaacagggagaagatcgagaagatcctgaccttccgaatcccctactacgtgggccctctggcccgaggcaactctcgattcgcctggatgacccgcaagtctgaggagaccatcaccccttggaacttcgaggaggtcgtggacaagggcgcctctgcccagtcattcatcgagcggatgaccaacttcgacaagaacctgcccaacgagaaggtgctgcctaagcattctttgctgtacgagtacttcaccgtgtacaacgagctgaccaaggtgaagtacgtgaccgagggcatgcgcaagcctgccttcctgtctggcgagcagaagaaggccatcgtggacctgttgttcaagaccaaccggaaggtgaccgtgaagcagctgaaggaggactacttcaagaagatcgagtgcttcgactctgtggagatcagcggcgtggaggaccgcttcaacgcctctctgggcacctaccatgacctgttgaagatcatcaaggacaaggacttcctggacaacgaggagaacgaggacatcctggaggacatcgtgctgaccttgaccctgttcgaggaccgggagatgatcgaggagcggctgaagacctacgcccatctgttcgacgacaaggtgatgaagcagctgaagcggagaaggtacaccggctggggcagactgtctagaaagctgatcaacggcatccgcgacaagcagtctggcaagaccatcctggacttcctgaagtctgacggcttcgccaaccggaacttcatgcagctgatccatgacgactctctgaccttcaaggaggacatccagaaggcccaggtgtctggccagggcgactctctgcatgagcatatcgccaacctggccggctctcccgccatcaagaagggcatcctgcagaccgtgaaggtggtcgacgagctggtgaaggtcatgggcaggcataagcccgagaacatcgtgatcgagatggcccgcgagaaccagaccacccagaagggccagaagaactctcgggagagaatgaagaggatcgaggagggcatcaaggagctgggctctcagatcctgaaggagcatcctgtggagaacacccagctgcagaacgagaagctgtacctgtactacctgcagaacgggcgggacatgtacgtggaccaggagctggacatcaacagactctctgactacgacgttgaccatatcgtgcctcagagcttcctgaaggacgactctatcgacaacaaggtgctgacccgctctgacaagaaccggggcaagtctgacaacgtgccttctgaggaggtggtgaagaagatgaagaactactggcgccagctgcttaacgccaagctgatcacccagagaaagttcgacaacctgaccaaggccgagcgaggcggcctctctgagctggacaaggccggcttcatcaagagacagctggtggagaccagacagatcaccaagcatgtggcccagatcctggactctagaatgaacaccaagtacgacgagaacgacaagctgatccgggaggtgaaggtgatcaccctgaagtctaagctggtcagcgacttccgcaaggacttccagttctacaaggtgagagagatcaacaactaccatcacgcccatgacgcctacctgaacgccgtggtcggcaccgccttgatcaagaagtaccctaagctggagtctgagttcgtgtacggcgactacaaggtgtacgacgtgagaaagatgatcgccaagtctgagcaggagatcggcaaggccaccgccaagtacttcttctactctaacatcatgaacttcttcaagaccgagatcaccctggccaacggcgagatcagaaagcggcccctgatcgagaccaacggcgagaccggcgagatcgtgtgggacaagggcagagacttcgccaccgtcagaaaggtcctgtctatgccccaggtgaacatcgtgaagaagaccgaggtgcagaccggcggcttctctaaggagtctatcctgcccaagcggaacagcgacaagctgatcgccagaaagaaggactgggaccccaagaagtacggcggcttcgactctcccaccgtggcctactctgtcctggtggtcgccaaggtcgagaagggcaagtctaagaagctgaagtctgtgaaggagctgctcggcatcaccatcatggagagaagctctttcgagaagaaccctatcgacttcctggaggccaagggctacaaggaggtgaagaaggacctgatcatcaagctgcccaagtactctctgttcgagctggagaacggccggaagagaatgctggcctctgccggcgagctgcagaagggcaacgagctggccttgccttctaagtacgtgaacttcttgtacctggcctctcactacgagaagctgaagggctctcccgaggacaacgagcagaagcagctgttcgtggagcagcataagcattacctggacgagatcatcgagcagatcagcgagttctctaagcgggtgatcctggccgacgccaacctggacaaggtcctgtctgcctacaacaagcatagagacaagcccatcagagagcaggccgagaacatcatccacctgttcaccctgaccaacctgggcgcccccgccgccttcaagtacttcgacaccaccatcgacagaaagcggtacaccagcaccaaggaggtgctcgacgccaccctgatccatcagtctatcaccggcctgtacgagaccagaatcgacctgagccagctgggcggcgactga

SEQ ID NO: 2 - DNA, 4107 bp, Synthetic construct
  source 1..4107: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctactggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactag

SEQ ID NO: 3 - DNA, 4107 bp, Synthetic construct
  source 1..4107: mol_type = other DNA
atggacaagaagtactctatcggcctggacatcggcaccaacagcgtgggctgggccgtcatcaccgacgagtacaaggtgccttctaagaagttcaaggtgctgggcaacaccgaccgccattctatcaagaagaacctgatcggcgccctgctgttcgactctggcgagaccgccgaggccaccagactgaagcggaccgcccgacgccgatacaccagacggaagaacagaatctgctaccttcaggagatcttcagcaacgagatggccaaggtggacgactctttcttccatcgcctggaggagagcttcctggtggaggaggacaagaagcatgagcgccatcctatcttcggcaacatcgtggacgaggtggcctaccatgagaagtaccctaccatctaccatctgaggaagaagctggtggactctacggacaaggccgacctgagacttatctacctggccctggcccatatgatcaagttccggggccatttcctcatcgagggcgacctcaaccccgacaacagcgacgtggacaagctgttcatccagttggtgcagacctacaaccagcttttcgaggagaaccccatcaacgcctctggcgtggacgccaaggccatcctgagtgcccgcctgtctaagagccgcagacttgagaacctgatcgcccagctgcccggcgagaagaagaacggcctgttcggcaaccttatcgccctgtctctgggccttacccctaacttcaagtctaacttcgacctggccgaggacgccaagctgcagcttagcaaggacacctacgacgacgacttggacaacctgcttgcccagatcggcgaccagtacgccgacctgttcctggccgccaagaacttgagcgacgccatcctgcttagcgacatcctgagagtcaacaccgagatcaccaaggcccctctgtctgccagcatgatcaagcggtacgacgagcatcaccaggacctgaccctgttgaaggccctcgtgcgacagcagctgcctgagaagtacaaggagatcttctttgaccagagcaagaacggctacgccggctacatcgacggcggcgcctctcaggaggagttctacaagttcatcaagcccatcctggagaagatggacggcaccgaggagcttctggtcaagctgaacagggaggacctgcttaggaagcagcgcaccttcgacaacggctcaatccctcatcagatccacctgggcgagttgcatgccatcctcagacgccaggaggacttctaccccttcctgaaggacaacagggagaagatcgagaagatcctgaccttccgaatcccctactacgtgggccctctggcccgaggcaactctcgattcgcctggatgacccgcaagtctgaggagaccatcaccccttggaacttcgaggaggtcgtggacaagggcgcctctgcccagtcattcatcgagcggatgaccaacttcgacaagaacctgcccaacgagaaggtgctgcctaagcattctttgctgtacgagtacttcaccgtgtacaacgagctgaccaaggtgaagtacgtgaccgagggcatgcgcaagcctgccttcctgtctggcgagcagaagaaggccatcgtggacctgttgttcaagaccaaccggaaggtgaccgtgaagcagctgaaggaggactacttcaagaagatcgagtgcttcgactctgtggagatcagcggcgtggaggaccgcttcaacgcctctctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctactggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactga

SEQ ID NO: 4 - DNA, 3687 bp, Synthetic construct
  source 1..3687: mol_type = other DNA
atgagtaagctggagaagttcaccaactgctacagcctgagcaagaccctgaggtttaaggccatccctgtgggcaagacccaggagaacatcgacaacaagcgactcctggtggaggacgagaagagggccgaggactacaagggcgtcaagaagctgcttgaccgctactacctgagtttcatcaacgacgtgctccatagcatcaagctgaagaaccttaacaactacatcagcctgtttcggaagaagacccggaccgagaaggagaataaggagcttgagaacctggagatcaacctccggaaggagatcgccaaggccttcaagggcaacgagggctacaagtccctgttcaagaaggacatcatagagaccatcctgcccgagttccttgacgacaaggacgagatcgccctggtgaacagcttcaacggcttcaccaccgccttcaccggcttcttcgacaaccgggagaacatgtttagcgaggaggccaagtctaccagcatcgccttcaggtgcatcaacgagaaccttactcggtacatcagcaacatggacatcttcgagaaggtggacgcgatcttcgacaagcatgaggtgcaggagatcaaggagaagatcctcaacagcgactacgacgtcgaggacttcttcgagggggagttcttcaacttcgtgcttacccaggaaggcatcgacgtgtacaacgccatcatcggcggcttcgtgaccgagtctggcgagaagatcaagggcctgaacgagtacatcaatctctacaatcagaagaccaaacagaagcttcccaagttcaaacccctgtacaagcaggtgctgtctgaccgggagtctcttagcttctacggcgagggatacacctctgacgaggaggtgctggaggtattccggaacaccctgaataagaacagtgagatcttcagctctatcaagaaactggagaagcttttcaagaattttgacgagtacagcagtgctggcatcttcgtgaaaaacggcccagccatcagtaccatctctaaggacatcttcggcgagtggaacgtgatcagggacaagtggaacgccgagtacgacgacatccaccttaagaagaaggcagtcgtgaccgagaagtacgaggacgacagacggaagtctttcaagaagatcggaagcttcagcttggagcagctccaagagtacgcagacgctgacctgtccgtggtggagaagctgaaggagattattatccagaaggtggacgagatttacaaggtgtacggctctagcgagaagcttttcgacgccgacttcgtgctggagaaatctctgaagaaaaacgacgccgtggtggccattatgaaggacctgctggactctgtgaagagcttcgagaactacatcaaggccttcttcggcgaaggaaaggagaccaacagagacgagagcttctacggcgacttcgtgctggcctacgacatcctgctgaaggtggaccacatttacgacgccattagaaactacgtgacccagaagccttacagcaaggacaaattcaagctttacttccagaacccccagttcatggggggctgggacaaggacaaggagaccgactacagagccaccatccttagatacggatctaagtactaccttgccatcatggacaagaagtacgccaagtgcctgcagaagattgacaaggacgacgtgaacggaaactacgagaagattaactacaagctgctgcccggccctaacaagatgcttcccaaggtgttcttcagcaagaagtggatggcctactacaaccctagtgaggacattcagaagatctacaagaatggcaccttcaagaagggcgacatgttcaaccttaacgactgccacaagctgatcgacttctttaaggacagcatcagtagataccccaagtggtccaacgcctacgacttcaacttctctgagacagagaagtataaggacattgctggtttttacagggaggtggaggagcagggctacaaggtgagcttcgagtctgccagcaagaaggaggtggacaaactggtggaggagggcaagctgtacatgtttcaaatttacaataaggacttcagcgacaagagccacggcactcctaatctgcacaccatgtacttcaaactgcttttcgacgagaacaatcatggccagatcagactgtccggcggcgccgagttgttcatgagaagagccagcctgaagaaggaggagctggtggtgcaccccgccaattctcccatcgctaacaagaaccccgacaaccccaagaagactaccacccttagctacgacgtatacaaggacaagcggtttagcgaggaccagtacgagctgcacatccccatcgccatcaacaagtgcccgaagaatattttcaagatcaacactgaggtgagagtcctgctgaagcacgacgacaacccctacgtgatcggcatcgacagaggcgagagaaacctcctgtacatcgtggtggtggacggcaagggcaatatcgtggagcagtacagccttaacgagattatcaacaacttcaacggcatcagaattaagaccgactaccactccctgctggacaagaaggaaaaggagagattcgaggccaggcagaactggacaagcattgagaacatcaaggagctgaaggccggctacatcagccaagttgtgcacaagatttgcgagctggtggagaaatacgacgccgtgatcgccttggaggacctcaacagcggcttcaagaactctcgggtgaaggtggagaagcaggtgtaccagaagttcgagaagatgctgattgacaagctgaactatatggtggacaagaagagcaacccctgcgccacaggcggcgctctgaagggctaccaaatcaccaacaagttcgagagcttcaagtcaatgtctacccagaacggcttcatcttctacatccctgcctggcttacctccaagatcgatccgagcaccggctttgtgaatttgcttaagactaagtacacttctatcgccgactccaaaaagttcattagctctttcgacagaatcatgtatgtgcccgaagaggacctgttcgaatttgccctcgactacaagaatttctccaggactgacgctgactatatcaagaagtggaagctgtacagctatggcaacagaatccgaatcttccgcaacccaaagaagaacaatgtcttcgattgggaggaggtgtgcttgactagcgcctacaaggagctgttcaacaagtatggcattaactatcagcaaggcgacatccgggcactgctgtgtgagcaatctgacaaagccttttacagctcttttatggctcttatgtctctcatgttgcagatgagaaacagcatcaccggcagaactgacgtggacttcctcatttctcccgtgaagaactccgacggcatcttctacgactctagaaactacgaagcccaggagaacgccatcctgcccaaaaacgccgacgccaacggcgcctacaacatcgccagaaaggtgctgtgggccatcgggcagttcaagaaagccgaggacgagaagcttgacaaagtgaagatcgccatcagcaacaaggagtggctggagtacgcccagaccagcgtgaagcactga

SEQ ID NO: 5 - DNA, 2925 bp, Synthetic construct
  source 1..2925: mol_type = other DNA
atgagcgaggccagcatcgagaagaagaaatctttcgccaagggcatgggcgtgaagagcaccctggtgtctggcagcaaggtgtacatgaccaccttcgccgagggctctgacgcccggctggagaagatagttgagggcgacagcatccggagcgtgaacgagggcgaggccttctcagccgagatggccgacaagaacgccggctacaagattgggaacgcgaagtttagtcatcccaagggctacgccgtggtggccaacaaccccctgtacaccggccccgtgcagcaggacatgctgggcctgaaggagaccctggagaagaggtacttcggcgagtctgccgacggcaacgacaacatctgcatccaggtgatccacaacatcctggacatcgagaagatcctggccgagtacatcaccaacgccgcctacgccgtgaacaacatcagcggcctggacaaggacattatcggctttggcaagttttctaccgtgtacacctacgacgagttcaaagaccctgaacatcatcgggccgccttcaacaacaacgataagctgattaacgccatcaaggcccagtacgacgagttcgacaacttcctggacaacccacgactgggctactttggccaggctttcttcagcaaggagggaagaaactacatcatcaactacggaaacgagtgctatgacattctcgccctcctgtctggcctgagacactgggtcgtacacaacaacgaggaggagtctcggattagcagaacctggctgtacaacctggataaaaacctcgacaacgagtacatctctacccttaactacctgtacgacagaatcaccaacgagctcaccaattctttctctaagaactctgccgccaacgtgaactacattgccgagaccctggggattaaccccgccgagttcgccgagcagtacttcagattcagcattatgaaggagcagaagaacctgggctttaacatcaccaagctgagagaggtgatgctggacaggaaggacatgagcgagatccgaaagaaccataaggtgttcgacagcatcaggaccaaggtgtacaccatgatggacttcgtcatctacaggtactacatcgaggaggacgccaaggtggctgcggcaaacaagagcctgcctgataacgagaagagcctgtctgagaaggacatcttcgtgatcaatctgagaggttctttcaacgacgaccaaaaggacgccctgtactatgacgaagccaacaggatttggcgaaagctggagaacatcatgcacaacatcaaggagttcaggggcaataagacacgcgagtacaagaagaaggacgcccccagactgcccagaattctgcccgccggcagggatgtgagcgccttctctaagctgatgtatgccctgaccatgtttctggacggcaaagagatcaacgacctgttgaccaccttgatcaacaaatttgacaacatccagagcttcctgaaggtgatgcccttgatcggcgtgaacgccaagttcgtggaggagtacgccttcttcaaagactctgccaagattgccgacgaactgagactgatcaagtctttcgccaggatgggagagcccatcgccgatgccaggagggccatgtacatcgatgccatccggatcctgggcaccaacctgtcttacgacgagctgaaagccctggccgacaccttttccctggacgagaacggcaacaagcttaagaagggcaagcacggcatgagaaacttcatcatcaacaacgtgatcagcaacaagaggttccattacctgatcaggtacggcgaccccgcccatctgcacgagattgccaagaacgaagccgtggtgaagttcgtgctgggccggattgctgacatccagaagaagcaaggccagaacggcaagaaccagatcgacaggtactacgaaacctgtattggcaaggacaagggcaagagcgtgtctgagaaggtggacgccctcaccaagatcattaccggcatgaactacgaccagttcgacaagaagaggtctgtgattgaagacaccggacgggagaacgccgagagagaaaagttcaagaagattatcagcttgtacctgaccgtgatttaccatatcctgaagaacatcgtgaacatcaacgcccggtacgtgatcggcttccactgcgtggagcgggacgcccagctgtacaaggagaagggctacgatatcaatctgaaaaagctggaggagaagggcttctccagcgtgaccaagctgtgcgccggcatcgacgagaccgcccccgacaagcggaaagacgtggagaaggagatggccgagagggccaaggagtctatcgactctctggagtctgccaaccccaagctttatgcgaattacatcaagtacagcgacgagaaaaaggccgaggagtttaccaggcagatcaatcgggagaaggccaaaaccgccctgaacgcctacctgcgcaacaccaagtggaacgtgattatccgggaggacctgctgcggattgacaacaagacctgcaccttgttcaggaacaaggccgtgcatctggaggtggccaggtacgtgcacgcctacattaacgacatcgccgaggtgaattcctactttcagctgtaccactacataatgcaaagaatcatcatgaacgaaaggtacgagaagagcagcggcaaggtgagcgagtacttcgacgccgtgaacgacgagaagaaatacaacgatcggctcctgaagctgctgtgtgtgccctttggctactgcatccctagattcaagaacctttctatcgaggccctgttcgaccggaacgaggccgccaagtttgataaggagaaaaagaaggtgagcggcaacagcggcagcggctga

SEQ ID NO: 6 - DNA, 3687 bp, Synthetic construct
  source 1..3687: mol_type = other DNA
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcactag

SEQ ID NO: 7 - DNA, 2925 bp, Synthetic construct
  source 1..2925: mol_type = other DNA
atgagcgaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggataa

SEQ ID NO: 8 - DNA, 294 bp, Homo sapiens
  source 1..294: mol_type = genomic DNA
atgagggagatcgtgcacatccaggccggccagtgcggcaaccagatcggggccaagttctgggaagtcatcagtgatgagcatggcatcgaccccagcggcaactacgtgggcgactcggacttgcagctggagcggatcagcgtctactacaacgaggcctcttctcacaagtacgtgcctcgagccattctggtggacctggaacccggaaccatggacagtgtccgctcaggggcctttggacatctcttcaggcctgacaatttcatctttggtccacatctgctttga

SEQ ID NO: 9 - DNA, 777 bp, Escherichia coli
  source 1..777: mol_type = genomic DNA
atgattgttttacccagacgcctgtcagacgaggttgccgatcgtgtgcgggcgctgattgatgaaaaaaacctggaagcgggcatgaagttgcccgctgagcgccaactggcgatgcaactcggcgtatcacgtaattcactgcgcgaggcgctggcaaaactggtgagtgaaggcgtgctgctcagtcgacgcggcggcgggacgtttattcgctggcgtcatgacacatggtcggagcaaaacatcgtccagccgctaaaaacactgatggccgatgatccggattacagtttcgatattctggaagcccgctacgccattgaagccagcaccgcatggcatgcggcaatgcgcgccacacctggcgacaaagaaaagattcagctttgctttgaagcaacgctaagtgaagacccggatatcgcctcacaagcggacgttcgttttcatctggcgattgccgaagcctcacataacatcgtgctgctgcaaaccatgcgcggtttcttcgatgtcctgcaatcctcagtgaagcatagccgtcagcggatgtatctggtgccaccggttttttcacaactgaccgaacaacatcaggctgtcattgacgccatttttgccggtgatgctgacggggcgcgtaaagcaatgatggcgcaccttagttttgttcacaccaccatgaaacgattcgatgaagatcaggctcgccacgcacggattacccgcctgcccggtgagcataatgagcattcgagggagaaaaacgcatga

SEQ ID NO: 10 - DNA, 777 bp, Synthetic construct
  source 1..777: mol_type = other DNA
atgatagtattgccccgacgacttagtgacgaggtcgcagatcgagtcagagcccttattgatgagaaaaaccttgaagcaggaatgaagcttcccgcagaacggcagctcgcgatgcaacttggggtgtcccgcaactccttgcgcgaagcactcgcgaaactggtgagcgaaggtgtgctcttgagtcgcaggggcggtggtacattcatcaggtggagacatgacacgtggtcagagcaaaacattgttcaacctctcaaaactctcatggcagatgatcctgactattcatttgacattctcgaggcccggtacgccatagaggcgagcactgcgtggcatgccgccatgcgagccacgccgggcgataaggagaagatacaactctgcttcgaggccaccctgtcagaggatcctgacattgcgagtcaggcagatgttcgattccacctcgcaatagcagaagcctctcacaacatcgtcctgttgcagactatgcgcggattttttgatgtcttgcaatccagcgtcaaacactcacgccaaaggatgtacttggtcccacctgtgttctcccaactgactgagcagcaccaagctgtaatcgacgcaatttttgcgggcgacgctgatggtgcaaggaaggcaatgatggctcatcttagctttgtccacacaactatgaagagatttgatgaagaccaagcaaggcatgcgagaataacaaggctgcctggagaacacaatgaacacagtagagaaaaaaatgcttga

SEQ ID NO: 11 - DNA, 777 bp, Synthetic construct
  source 1..777: mol_type = other DNA
atgatcgtgctccccagaaggctgtccgacgaggtggccgacagagtcagagccctgatcgacgagaagaacctggaggccggcatgaagctgcccgccgagcgacagctggccatgcagctgggcgtgagcagaaacagcctgcgcgaggccctggccaagctcgtgtctgagggcgtcctgctgtctagaagaggaggcggaaccttcatccgctggagacacgacacctggagcgagcaaaatatcgtgcagcctctgaagaccctgatggcggacgaccccgactatagcttcgacatactggaggccaggtacgccattgaagcatccaccgcgtggcacgccgctatgagggccacccccggagacaaggagaagatccagctgtgcttcgaggccactctgagcgaggaccctgacattgccagccaggccgacgtgaggttccacctggccatcgctgaggccagccacaacatcgtgctgctgcagaccatgagaggcttcttcgacgtcctgcagagcagcgtgaagcactcaagacagagaatgtacctcgtcccccctgtgttctcccagttgacagagcagcaccaggccgtgatagacgctatctttgccggagatgccgacggcgccagaaaggccatgatggcccacctgagcttcgtgcataccaccatgaagcgcttcgacgaggaccaggctagacacgccagaatcaccagactgcccggcgagcacaacgagcactccagagagaagaacgcctga

(19) United States
(12) Patent Application Publication
(10) Pub. No.: US 2024/0301402 A1
(43) Pub. Date: Sep. 12, 2024

(54) PROTEIN EXPRESSION

(71) Applicant: Cambridge Enterprise Limited, Cambridge (GB)

(72) Inventor: Emmanouil Metzakopian, Cambridge (GB)

(21) Appl. No.: 18/677,399

(22) Filed: May 29, 2024

Related U.S. Application Data
(63) Continuation of application No. PCT/GB2022/053106, filed on Dec. 6, 2022.

(30) Foreign Application Priority Data
Dec. 6, 2021 (GB) 2117583.1

(51) Int. Cl. C12N 15/10; C12N 5/071; C12N 5/074; C12N 9/22

(52) U.S. Cl. CPC C12N 15/1079; C12N 5/0686; C12N 5/0696; C12N 9/22; C12N 2506/45

(57) ABSTRACT

This invention relates to a method for codon optimising a target nucleic acid sequence for expression in a host cell. The invention also relates to codon optimised nucleic acids for improved expression in a host cell, and to vectors and host cells comprising codon optimised nucleic acids.

### CROSS-REFERENCE TO RELATED APPLICATIONS

This application is a continuation of International (PCT) Patent Application No. PCT/GB2022/053106, filed Dec. 6, 2022, which claims the benefit of and priority to United Kingdom Patent Application No. 2117583.1, filed on Dec. 6, 2021, the disclosures of each of which are hereby incorporated by reference in their entireties for all purposes.

### SEQUENCE LISTING

The instant application contains a Sequence Listing XML which has been submitted electronically and is hereby incorporated by reference in its entirety. Said XML copy, created on May 29, 2024, is named BBIO-009USWOC1_SL.xml, and is 38,673 bytes in size.

### FIELD OF THE INVENTION

This invention relates to a method for codon optimising a target nucleic acid sequence for expression in a host cell. The invention also relates to codon optimised nucleic acids for improved expression in a host cell, and to vectors and host cells comprising codon optimised nucleic acids.

### BACKGROUND OF THE INVENTION

A codon is a trinucleotide sequence of DNA or RNA which encodes a specific amino acid or signals the termination of translation (“termination” or “stop” codon). Degeneracy exists within the genetic code because more codon sequences exist than there are amino acids or stop codons. In fact, 18 of the 20 common amino acids are encoded by multiple ‘synonymous codons’ (i.e. different codons which encode the same amino acid). Codon usage can vary significantly between species: different species typically display “bias” towards certain codons and some species use particular codons only very rarely or not at all. When a gene of interest contains codons that are rarely used by a host, that gene encounters stalled translation within a cell from that host, thereby reducing the efficiency of expression or preventing expression entirely. Codon optimisation approaches account for differences in codon biases between species and are designed to improve the codon composition of a target nucleic acid sequence by replacing codons that are rarely used by the host with synonymous codons that are used with a higher frequency by the host and are thus “preferred” by the host.

Codon usage has recently been spotlighted as a key determinant of translation elongation rates and co-translational protein folding, with host preferred codons enhancing translational efficiency and folding fidelity. The unequal usage of synonymous codons, referred as “codon bias” and the universal nature of this bias, from yeast to humans, suggests the existence of a secondary code within the more familiar genetic code. This secondary code is emerging as a major regulator of translational speed and co-translational protein folding and thereby a significant determinant of the cellular levels of specific proteins.

To identify the codon biases of a particular host, the frequency of codon usage is typically determined across several hundred or thousand coding DNA sequences (CDS). To codon optimise a gene of interest, codons within the gene that are present at low frequency (or not at all) in the host (which may be referred to as “non-preferred codons”) are replaced with synonymous codons that are more commonly used by the host (which may be referred to as “preferred codons”). Codon optimisation aims to improve the expression efficiency of genes of interest without altering the sequence of the encoded proteins.

Although well-established codon optimisation methods are known in the art, some genes remain challenging to express and, in some instances, codon optimised genes do not achieve sufficiently high expression levels or are unable to maintain sufficient expression levels over time.

For example, human induced pluripotent stem cells (hiPSC/iPSCs) represent a powerful tool for research with the potential to differentiate into multiple cell types. However, application of these cells in genome wide genetic screens using the CRISPR (clustered regularly interspaced short palindromic repeats)-Cas (CRISPR associated protein) gene editing system has been prevented by the inability of these cells to efficiently express Cas proteins, e.g. Cas9, despite the genes encoding these proteins being codon optimised for expression in human cell lines. The mechanisms by which Cas genes are silenced in differentiated cell types derived from iPSCs are currently unknown.

There exists an urgent and unmet need for improved codon optimisation methods that enable the efficient expression of target nucleic acid sequences in host cells.

### SUMMARY OF THE INVENTION

The inventors have developed a novel method for codon optimising a target nucleic acid sequence for expression in a host cell. According to the invention, codon optimisation utilises the codon usage frequency of a gene encoding a protein that is highly expressed by the host cell or the codon usage frequency of a gene encoding a protein that is highly expressed in a cell from the same species as the host cell. Codons within the target nucleic acid that are used with low frequency by the gene encoding the highly expressed protein are replaced with synonymous codons that are used with high frequency by the gene encoding the highly expressed protein.

The current “gold standard” for codon optimising target nucleic acids is based upon species level codon biases which are derived from hundreds or thousands of coding sequences. Surprisingly, the inventors found that codon optimising target nucleic acid sequences based on the codon biases of genes encoding highly expressed proteins significantly improved the expression efficiency compared to corresponding nucleic acids optimised using the current gold standard. Codon optimisation according to the invention achieves high level and sustained expression, even in cell types that do not typically express the gene comprising the nucleic acid sequence on which the codon optimisation was based.

Importantly, codon optimisation according to the invention achieves high level and sustained protein expression in iPSCs and in differentiated cell lines derived from iPSCs, which significantly improves the potential application of these cells in research.

The invention provides a method for codon optimising a target nucleic acid sequence for expression in a host cell comprising altering the codon usage frequency of the target nucleic acid sequence based on the codon usage frequency of a gene encoding a protein that is highly expressed in the host cell or the codon usage frequency of a gene encoding a protein that is highly expressed in a cell from the same species as the host cell.

In some embodiments, the method comprises substituting one or more non-preferred codons within the target nucleic acid sequence with preferred synonymous codons, wherein: (a) non-preferred codons are codons used with low frequency by the gene encoding the highly expressed protein; and (b) preferred codons are codons used with high frequency by the gene encoding the highly expressed protein.

In some embodiments, non-preferred codons are codons used with lower frequency by the gene encoding the highly expressed protein than would be expected if each synonymous codon was used at random.

In some embodiments, non-preferred codons are used by the gene encoding the highly expressed protein with a frequency of less than 50%, less than 45%, less than 40%, less than 35%, less than 33%, less than 30%, less than 25%, less than 20%, less than 16%, less than 15%, less than 10%, less than 5%, or 0%.

In some embodiments, preferred codons are codons used with higher frequency by the gene encoding the highly expressed protein than would be expected if each synonymous codon was used at random.

In some embodiments, preferred codons are used by the gene encoding the highly expressed protein with a frequency of at least 50%, at least 55%, at least 60%, at least 65%, at least 70%, at least 75%, at least 80%, at least 85%, at least 90%, at least 95%, or 100%.

In some embodiments, the method comprises replacing at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of non-preferred codons within the target nucleic acid with preferred synonymous codons.

In some embodiments, the method comprises replacing all non-preferred codons within the target nucleic acid that are used with a frequency of 0% by the gene encoding the highly expressed protein with a preferred synonymous codon.

In some embodiments, the method comprises replacing all non-preferred codons with a preferred synonymous codon in a region of the target nucleic acid that encodes the N-terminal region of a protein.

In some embodiments, the method comprises replacing all non-preferred codons with a preferred synonymous codon in the 5′ region of the target nucleic acid, optionally the first at least 100, at least 200, at least 300, at least 400, at least 500, at least 600, at least 700, at least 800, at least 900, or at least 1000 codons starting from the 5′ end of the target nucleic acid.

In some embodiments, the protein that is highly expressed is a housekeeping protein or a cell marker protein. In some embodiments, the protein that is highly expressed is selected from GAPDH, β-tubulin, β-actin, and tubulin III. In some embodiments, the protein that is highly expressed is tubulin III. In some embodiments, the one or more non-preferred codons are selected from: alanine codons GCA, GCG and GCT; arginine codons AGA and CGT; cysteine codon TGT; glutamine codon CAA; isoleucine codon ATA; leucine codons CTA and TTA; lysine codon AAA; proline codon CCG; serine codon TCC; threonine codons ACA, ACG and ACT; tyrosine codon TAT; valine codons GTA and GTT; and stop codons TAA and TAG.

In some embodiments, the one or more non-preferred codons are selected from: asparagine codon AAT; aspartic acid codon GAT; glutamic acid codon GAA; glycine codons GGA, GGG and GGT; histidine codon CAC; isoleucine codon ATT; leucine codons CTC, CTT and TTG; phenylalanine codon TTT; proline codon CCA; serine codons TCA and TCG; and valine codon GTC.

In some embodiments, preferred codons are selected from: alanine codon GCC; cysteine codon TGC; glutamine codon CAG; lysine codon AAG; threonine codon ACC; tyrosine codon TAC; and the stop codon TGA.

In some embodiments, preferred codons are selected from: arginine codons AGG, CGA, CGC and CGG; asparagine codon AAC; aspartic acid codon GAC; glutamic acid codon GAG; glycine codon GGC; histidine codon CAT; isoleucine codon ATC; leucine codon CTG; phenylalanine codon TTC; proline codons CCC and CCT; serine codons AGC, AGT and TCT; and valine codon GTG.

In some embodiments, the host cell is selected from a human cell, a bacterial cell, a yeast cell and a fungal cell. In some embodiments, the host cell is a human cell. In some embodiments, the host cell is a HEK293 cell. In some embodiments, the host cell is a human induced pluripotent stem cell (iPSC). In some embodiments, the host cell is a differentiated cell derived from an iPSC, optionally wherein the host cell is selected from an iPSC derived neuron such as a cortical neuron, dopaminergic neuron or a motor neuron, an iPSC derived macrophage, an iPSC derived cardiomyocytes, and an iPSC derived hepatocyte.

In some embodiments, the target nucleic acid encodes a Cas protein, optionally wherein the Cas protein is selected from Cas9, Cas12a and Cas13Rx.

The invention also provides a nucleic acid comprising a nucleic acid sequence that has been codon optimised by the method of the invention.

The invention also provides a codon optimised nucleic acid for improved expression in a host cell wherein the codon usage frequency of the nucleic acid corresponds to the codon usage frequency of a gene encoding a protein that is highly expressed by the host cell or the codon usage frequency of a gene encoding a protein that is highly expressed in a cell from the same species as the host cell.

In some embodiments, the codon optimised nucleic acid comprises a lower frequency of non-preferred codons than a non-optimised nucleic acid sequence encoding the same amino acid sequence.

In some embodiments, the codon optimised nucleic acid comprises a higher frequency of preferred codons than a non-optimised nucleic acid sequence encoding the same amino acid sequence.

The invention also provides a nucleic acid encoding Cas9 and comprising a nucleic acid sequence having at least 70%, at least 75%, at least 80%, at least 85%, at least 90%, at least 95% or 100% sequence identity to SEQ ID NO: 1 or SEQ ID NO: 3.

The invention also provides a nucleic acid encoding Cas12a and comprising a nucleic acid sequence having at least 70%, at least 75%, at least 80%, at least 85%, at least 90%, at least 95% or 100% sequence identity to SEQ ID NO: 4.

The invention also provides a nucleic acid encoding Cas13Rx and comprising a nucleic acid sequence having at least 70%, at least 75%, at least 80%, at least 85%, at least 90%, at least 95% or 100% sequence identity to SEQ ID NO: 5.

The invention also provides a vector comprising a nucleic acid according to the invention.

The invention also provides a host cell comprising a nucleic acid according to the invention or a vector according to the invention.

In some embodiments, the host cell is selected from a human cell, a bacterial cell, a yeast cell and a fungal cell. In some embodiments, the host cell is a human cell. In some embodiments, the host cell is a HEK293 cell. In some embodiments, the host cell is a human induced pluripotent stem cell (iPSC). In some embodiments, the host cell is a differentiated cell derived from an iPSC, optionally wherein the host cell is selected from an iPSC derived neuron such as a cortical neuron, dopaminergic neuron or a motor neuron, an iPSC derived macrophage, an iPSC derived cardiomyocytes, and an iPSC derived hepatocyte.

### DESCRIPTION OF THE DRAWINGS

FIG. 1. (A) Schematic of the PiggyBac (PB) transposon plasmid comprising the starting Cas9 (old-Cas9) sequence. (B) Cas9 and (C) GAPDH (glyceraldehyde-3-phosphate dehydrogenase) mRNA levels in iPSC Cas9 cells at 0 (iPSC), 10 and 20 days during differentiation to dopaminergic neurons. Cas9 mRNA decreases by approx. 60% by Day 20.

FIG. 2. (A) Schematic showing homology directed recombination used to knock-in Cas9 upstream of GAPDH. (B) Levels of Cas9 and GAPDH mRNA during iPSC differentiation to neurons in GapdhCas9-iNgn2 iPSC. (C) Levels of GAPDH mRNA in the GapdhCas9 and WT (no Cas9) iNgn2 cells during iPSC differentiation to neurons.

FIG. 3. (A) Western blot demonstrating Cas9 and GAPDH protein expression during differentiation of iPSCs to neurons. (B) Densitometry quantification of Cas9 protein levels from (A) in Bob-iNgn2 Gapdh-Cas9 cells during differentiation to neurons. (C) Schematic of fluorescence reporter construct used. (D) Flow cytometry plots showing dual fluorescence of the reporter construct (left) and showing loss of GFP fluorescence when in presence of Cas9 (right). (E) Cas9 cutting efficiency quantified by loss of GFP fluorescence reporter 4 days post reporter transduction.

FIG. 4. (A) Cas9 protein expression in the presence of either MG132 inhibitor to block proteasome degradation or Bafilomycin A1 (BafA1) to block the autophagy-lysosome pathway. (B) Cas9 protein expression in different media compositions. DMEM=Dulbecco's Modified Eagle Medium; NEAA=non-essential amino acid.

FIG. 5. Comparison between bacterial (E. coli) codon usage frequency (black bars) and human generic codon usage frequency (grey bars). Dashed boxes indicate codons with significant differences in usage frequency between humans and bacteria.

FIG. 6. Comparison between old-Cas9 codon usage frequency (black bars) and human generic codon usage frequency (grey bars). Solid boxes indicate optimised codons, dashed boxes indicate codons with random distribution.

FIG. 7. Comparison between tubulin III codon usage frequency (black bars) and human generic codon usage frequency (grey bars). Dashed boxes indicate codons that are not used by tubulin III; and triangles indicate codons with higher usage in tubulin III than in humans generally.

FIG. 8. Comparison between codon optimised Cas9 (CodOpt-Cas9; SEQ ID NO: 1) codon usage frequency (black bars) and human generic codon usage frequency (grey bars). Dashed boxes indicate codons that are not used by CodOpt-Cas9; and triangles indicate codons with higher usage in CodOpt-Cas9.

FIG. 9. Schematics of Old-Cas9 (left) and CodOpt-Cas9 (right) expression constructs.

FIG. 10. (A) Old-Cas9 and CodOpt-Cas9 mRNA levels in HEK293-Cas9 lines. (B) and (C) Protein levels of Old-Cas9 and CodOpt-Cas9 protein levels in HEK293 cells (B) and its quantification (C). (D) Cas9 editing efficiency in HEK293 cells using reporter plasmid.

FIG. 11. Formation of indels by Cas9 guided to edit a non-essential gene (ST6GALNAC6) in HEK-Cas9 and control lines. The graphs are TIDE profiles obtained by tracking indel formation at http://shinyapps.datacurators.nl/tide/. Y axis=% of sequences.

FIG. 12. (A) Old-Cas9 and CodOpt-Cas9 mRNA levels in Bob-iNgn2 Cas9 iPSCs cells generated using PiggyBac. (B) and (C) Western blot of Old-Cas9 and CodOpt-Cas9 protein levels in Bob-iNgn2 iPSCs cells together with its quantification (C).

FIG. 13. Western blot showing levels of Cas9 protein in Old-Cas9 and CodOpt-Cas9 Bob-iNgn2 iPSC lines during different time points of differentiation to neurons and its relative quantification.

FIG. 14. Schematics of Old-Cas9 (left); NOpt-Cas9 (middle) and CodOpt-Cas9 (right) expression constructs.

FIG. 15. Flow cytometry plots showing loss of GFP reporter fluorescence in Bob-iNgn2 iPSCs that harbour either the Old-Cas9, NOpt-Cas9 or CodOpt-Cas9. Editing efficiency of the Cas9 variants were assessed during differentiation to neurons (iPSC, Day 4 and Day 10).

FIG. 16. Quantification of Cas9 cutting efficiency by Old-Cas9, NOpt-Cas9, CodOpt-Cas9 or no Cas9 (WT) in Bob-iNgn2 iPSCs and at various time points of neuronal differentiation. The highlighted box emphasizes the differences in Cas9 editing as neurons differentiate in the protocol.

FIG. 17. Cas9 mRNA (A) and protein (B) levels produced by Old-Cas9, CodOpt-Cas9 or NOpt-Cas9 in Bob-iNgn2 iPSCs and during differentiation to neurons. Protein level quantification is represented in (C)-highlighted grey/black boxes emphasize the differences in Cas9 levels as neurons differentiate and mature.

FIG. 18. (A) Cas9 protein levels in iPSC derived hepatocytes that contain either Old-Cas9 or CodOpt-Cas9. (B) Quantification shows higher levels of CodOpt Cas9 in day 10 differentiated hepatoblastoma cells (grey/black boxes).

FIG. 19. Comparison between Cas12a codon usage frequency (black bars) and human generic codon usage frequency (grey bars). Dashed boxes indicate amino acids that are biased toward a particular codon.

FIG. 20. Comparison between codon optimised Cas12a codon usage frequency (black bars) and human generic codon usage frequency (grey bars).

FIG. 21. Comparison between Cas13Rx codon usage frequency (black bars) and human generic codon usage frequency (grey bars).

FIG. 22. Comparison between codon optimised Cas13Rx codon usage frequency (black bars) and human generic codon usage frequency (grey bars).

FIG. 23. Schematics of plasmids comprising LIDr optimised using the existing gold standard method based on human codon usage frequency (denoted “normal codon optimization”) and using the codon biases of tubulin III as described herein (denoted “novel codon optimization”).

FIG. 24. (A) Transfection efficiency of normal optimization and novel optimization plasmids in HEK293 and iPSC cells. (B) Western blot demonstrating LIDr (c-Myc) and GAPDH protein expression in Bob-iNgn2 iPSCs and HEK293 cells 5 days post-transfection. (C) Densitometry quantification of LIDr (c-myc) levels relative to Gapdh levels from (B) in Bob-iNgn2 iPSC and HEK293 cells. The existing gold standard codon optimization method is denoted “normal optimization” and the optimization method using the codon biases of tubulin III is denoted “novel optimization”.

### DETAILED DESCRIPTION

The invention is based on the surprising discovery that, by codon optimising a target nucleic acid sequence for expression in a host cell (or a cell from the same species as the host cell) based on the codon usage frequency of a gene encoding a protein that is highly expressed in the host cell (or the codon usage frequency of a gene encoding a protein that is highly expressed in a cell from the same species as the host cell), the expression of the target nucleic acid may be significantly improved. In particular, the inventors discovered that this approach achieves efficient and sustained expression of target nucleic acids that have previously been difficult to express, even when codon optimised using the current gold standard of codon optimisation based on species level codon biases.

Transgene expression is difficult in many host cell types. One example of host cells in which transgene expression can be challenging is cells derived from human induced pluripotent stem cells (hiPSC/iPSCs). iPSCs represent a powerful tool for research with the potential to differentiate into multiple cell types so there is a great desire to improve transgene expression in such cells. In recent years, a number of hiPSC based cell lines have been generated that allow controlled and quick differentiation into various cell types including macrophages (immune cells), cardiomyocytes (muscle cells) and neurons (nerve cells). These cell lines have applications in a wide range of research fields. For example, hiPSC derived neurons provide a powerful replacement to immortalized human cell lines and non-human primary neuronal cells for use in in vitro research, to understand neurodegenerative disorders, because they can be differentiated into specific neuronal sub-types that are found to be affected in these disorders. Several differentiation protocols have been optimized to generate specific neuronal subtypes such as cortical neurons, dopaminergic neurons and even motor neurons that can been utilized robustly to model Alzheimer's, Parkinson's or Motor Neuron Disease respectively.

Another powerful research tool is the CRISPR-Cas gene editing system which has revolutionized the molecular approaches that help in delineating cellular mechanisms, e.g. mechanisms of neuron degeneration. CRISPR-Cas9 genetic screens in multiple cell types have been essential in identifying novel cellular pathways and genetic targets that could aid in translational research. While a large number of these studies have relied on initiating a genome wide screen at iPSC/progenitor stage and extrapolating findings to iPSC-derived cell types, performing CRISPR-Cas9 screens in differentiated cells has been challenging, largely due to the inability to efficiently express Cas9 in iPSC derived cell lines. The mechanisms through which Cas9 is rendered inactive in iPSC derived differentiated cell types, including neurons, is currently unknown. The inability to efficiently express this key component of the CRISPR-Cas9 system dramatically limits the research potential of iPSC derived cell types.

Multiple approaches have been investigated in attempts to overcome Cas9 silencing during iPSC differentiation. Such approaches include integrating multiple copies of Cas9 into the genome using lentivirus/transposons; testing Cas9 expression under various mammalian expression promoters; and targeting Cas9 to specific locations in the genome, e.g. genomic safe harbour sites. Despite these efforts, Cas9 protein levels dramatically decrease in differentiated cells compared to levels observed in iPSCs. The inventors attempted to circumvent Cas9 silencing by inserting Cas9 at the site of a house-keeping gene (glyceraldehyde-3-phosphate dehydrogenase (GAPDH)) to help achieve continued expression. Despite successful knock-in at endogenous GAPDH gene, the housekeeping gene's promoter was unable to maintain constitutive expression of Cas9 protein during differentiation to neuronal cell types. Interestingly, despite a decrease in protein levels, mRNA levels of Cas9 remained detectable during differentiation suggesting that transcription and translation had become uncoupled.

The Cas9 gene typically used in experimental studies (herein “old-Cas9”) is derived from Streptococcus pyogenes and is codon optimised for expression in humans using human generic codon usage (which represents the current gold standard codon optimisation approach). Based on the uncoupling of Cas9 transcription and translation observed in iPSC derived cell lines, the inventors hypothesised that Cas9 may require further codon optimisation to be functional in differentiated cell types.

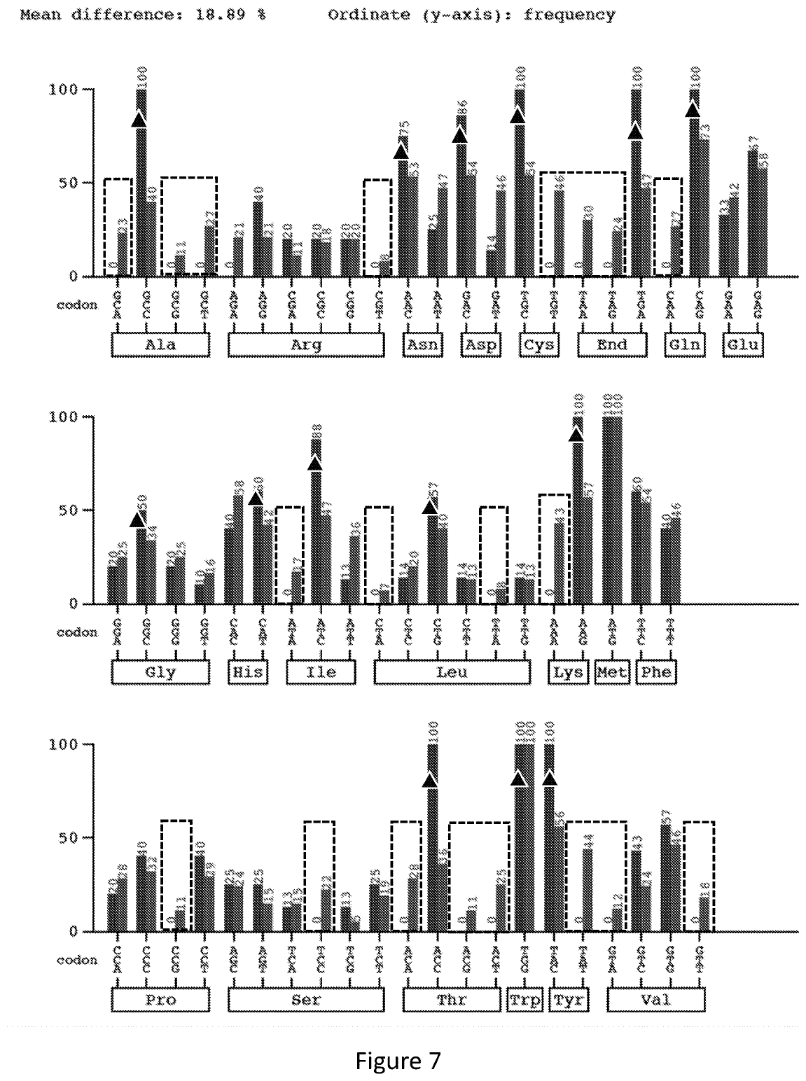

The inventors sought to identify whether genes that are highly expressed in iPSC derived differentiated cells exhibit specific codon biases by comparing the codon usage frequencies of tubulin III (Ensembl Transcript: TUBB3-208 ENST00000555576.5; SEQ ID NO: 8), a marker gene that is highly expressed in neuronal cells, with generic codon usage frequencies in humans (FIG. 7). Human generic codon usage is typically derived from tens of thousands of human coding DNA sequences (CDS). As used herein, human generic codon usage is derived from the Codon Usage Database provided by the Kazusa DNA Research Institute which is based on the codon usage of 93,487 human CDS (Nakamura, Y. et al. Nucleic acids research 2000 28(1):292). Unless otherwise specified, references herein to codon “usage frequency in humans” or “human generic codon usage” refers to the codon usage frequency stated in the Homo sapiens codon usage table in the Codon Usage Database by Kazusa.

The inventors found that tubulin III exhibits different codon biases for several codons compared to human generic codon usage. Surprisingly, tubulin III does not use several codons that are commonly used in humans, e.g. the cysteine codon TGT (46% usage frequency in humans); the lysine residue AAA (43% usage frequency in humans); and the tyrosine residue TAT (44% usage frequency in humans). In addition, tubulin III exhibits a strict preference for the alanine codon GCC (40% usage frequency in humans) and the threonine codon ACC (36% usage frequency in humans) which are used exclusively, despite the availability of three additional synonymous codons for each of these amino acids. Tubulin III also exhibits greater preference for specific codons, e.g. tubulin III uses the histidine residue CAT with higher frequency (60% usage frequency) than CAC (40% usage frequency), whereas CAC is preferred in human generic codon usage (58% usage frequency). The inventors suggest that high expression levels achieved by tubulin III in neuronal cells is due to these codon biases contributing to efficient expression in these cell types.

The inventors utilised tubulin III codon biases to generate a codon optimised version of Cas9 (CodOpt-Cas9) that more closely mirrors the codon usage frequency of tubulin III. The CodOpt-Cas9 sequence obtained after these alterations is represented by SEQ ID NO: 1:

(SEQ ID NO: 1)
ATGGACAAGAAGTACTCTATCGGCCTGGACATCGGCACCAACAGCGTGGGCTGGGCCGTCATCACCGACGAG
TACAAGGTGCCTTCTAAGAAGTTCAAGGTGCTGGGCAACACCGACCGCCATTCTATCAAGAAGAACCTGATCG
GCGCCCTGCTGTTCGACTCTGGCGAGACCGCCGAGGCCACCAGACTGAAGCGGACCGCCCGACGCCGATACA
CCAGACGGAAGAACAGAATCTGCTACCTTCAGGAGATCTTCAGCAACGAGATGGCCAAGGTGGACGACTCTT
TCTTCCATCGCCTGGAGGAGAGCTTCCTGGTGGAGGAGGACAAGAAGCATGAGCGCCATCCTATCTTCGGCA
ACATCGTGGACGAGGTGGCCTACCATGAGAAGTACCCTACCATCTACCATCTGAGGAAGAAGCTGGTGGACT
CTACGGACAAGGCCGACCTGAGACTTATCTACCTGGCCCTGGCCCATATGATCAAGTTCCGGGGCCATTTCCTC
ATCGAGGGCGACCTCAACCCCGACAACAGCGACGTGGACAAGCTGTTCATCCAGTTGGTGCAGACCTACAAC
CAGCTTTTCGAGGAGAACCCCATCAACGCCTCTGGCGTGGACGCCAAGGCCATCCTGAGTGCCCGCCTGTCTA
AGAGCCGCAGACTTGAGAACCTGATCGCCCAGCTGCCCGGCGAGAAGAAGAACGGCCTGTTCGGCAACCTTA
TCGCCCTGTCTCTGGGCCTTACCCCTAACTTCAAGTCTAACTTCGACCTGGCCGAGGACGCCAAGCTGCAGCTT
AGCAAGGACACCTACGACGACGACTTGGACAACCTGCTTGCCCAGATCGGCGACCAGTACGCCGACCTGTTCC
TGGCCGCCAAGAACTTGAGCGACGCCATCCTGCTTAGCGACATCCTGAGAGTCAACACCGAGATCACCAAGG
CCCCTCTGTCTGCCAGCATGATCAAGCGGTACGACGAGCATCACCAGGACCTGACCCTGTTGAAGGCCCTCGT
GCGACAGCAGCTGCCTGAGAAGTACAAGGAGATCTTCTTTGACCAGAGCAAGAACGGCTACGCCGGCTACAT
CGACGGCGGCGCCTCTCAGGAGGAGTTCTACAAGTTCATCAAGCCCATCCTGGAGAAGATGGACGGCACCGA
GGAGCTTCTGGTCAAGCTGAACAGGGAGGACCTGCTTAGGAAGCAGCGCACCTTCGACAACGGCTCAATCCC
TCATCAGATCCACCTGGGCGAGTTGCATGCCATCCTCAGACGCCAGGAGGACTTCTACCCCTTCCTGAAGGAC
AACAGGGAGAAGATCGAGAAGATCCTGACCTTCCGAATCCCCTACTACGTGGGCCCTCTGGCCCGAGGCAAC
TCTCGATTCGCCTGGATGACCCGCAAGTCTGAGGAGACCATCACCCCTTGGAACTTCGAGGAGGTCGTGGACA
AGGGCGCCTCTGCCCAGTCATTCATCGAGCGGATGACCAACTTCGACAAGAACCTGCCCAACGAGAAGGTGC
TGCCTAAGCATTCTTTGCTGTACGAGTACTTCACCGTGTACAACGAGCTGACCAAGGTGAAGTACGTGACCGA
GGGCATGCGCAAGCCTGCCTTCCTGTCTGGCGAGCAGAAGAAGGCCATCGTGGACCTGTTGTTCAAGACCAA
CCGGAAGGTGACCGTGAAGCAGCTGAAGGAGGACTACTTCAAGAAGATCGAGTGCTTCGACTCTGTGGAGAT
CAGCGGCGTGGAGGACCGCTTCAACGCCTCTCTGGGCACCTACCATGACCTGTTGAAGATCATCAAGGACAA
GGACTTCCTGGACAACGAGGAGAACGAGGACATCCTGGAGGACATCGTGCTGACCTTGACCCTGTTCGAGGA
CCGGGAGATGATCGAGGAGCGGCTGAAGACCTACGCCCATCTGTTCGACGACAAGGTGATGAAGCAGCTGA
AGCGGAGAAGGTACACCGGCTGGGGCAGACTGTCTAGAAAGCTGATCAACGGCATCCGCGACAAGCAGTCT
GGCAAGACCATCCTGGACTTCCTGAAGTCTGACGGCTTCGCCAACCGGAACTTCATGCAGCTGATCCATGACG
ACTCTCTGACCTTCAAGGAGGACATCCAGAAGGCCCAGGTGTCTGGCCAGGGCGACTCTCTGCATGAGCATAT
CGCCAACCTGGCCGGCTCTCCCGCCATCAAGAAGGGCATCCTGCAGACCGTGAAGGTGGTCGACGAGCTGGT
GAAGGTCATGGGCAGGCATAAGCCCGAGAACATCGTGATCGAGATGGCCCGCGAGAACCAGACCACCCAGA
AGGGCCAGAAGAACTCTCGGGAGAGAATGAAGAGGATCGAGGAGGGCATCAAGGAGCTGGGCTCTCAGAT
CCTGAAGGAGCATCCTGTGGAGAACACCCAGCTGCAGAACGAGAAGCTGTACCTGTACTACCTGCAGAACGG
GCGGGACATGTACGTGGACCAGGAGCTGGACATCAACAGACTCTCTGACTACGACGTTGACCATATCGTGCCT
CAGAGCTTCCTGAAGGACGACTCTATCGACAACAAGGTGCTGACCCGCTCTGACAAGAACCGGGGCAAGTCT
GACAACGTGCCTTCTGAGGAGGTGGTGAAGAAGATGAAGAACTACTGGCGCCAGCTGCTTAACGCCAAGCTG
ATCACCCAGAGAAAGTTCGACAACCTGACCAAGGCCGAGCGAGGCGGCCTCTCTGAGCTGGACAAGGCCGG
CTTCATCAAGAGACAGCTGGTGGAGACCAGACAGATCACCAAGCATGTGGCCCAGATCCTGGACTCTAGAAT
GAACACCAAGTACGACGAGAACGACAAGCTGATCCGGGAGGTGAAGGTGATCACCCTGAAGTCTAAGCTGG
TCAGCGACTTCCGCAAGGACTTCCAGTTCTACAAGGTGAGAGAGATCAACAACTACCATCACGCCCATGACGC
CTACCTGAACGCCGTGGTCGGCACCGCCTTGATCAAGAAGTACCCTAAGCTGGAGTCTGAGTTCGTGTACGGC
GACTACAAGGTGTACGACGTGAGAAAGATGATCGCCAAGTCTGAGCAGGAGATCGGCAAGGCCACCGCCAA
GTACTTCTTCTACTCTAACATCATGAACTTCTTCAAGACCGAGATCACCCTGGCCAACGGCGAGATCAGAAAGC
GGCCCCTGATCGAGACCAACGGCGAGACCGGCGAGATCGTGTGGGACAAGGGCAGAGACTTCGCCACCGTC
AGAAAGGTCCTGTCTATGCCCCAGGTGAACATCGTGAAGAAGACCGAGGTGCAGACCGGCGGCTTCTCTAAG
GAGTCTATCCTGCCCAAGCGGAACAGCGACAAGCTGATCGCCAGAAAGAAGGACTGGGACCCCAAGAAGTA
CGGCGGCTTCGACTCTCCCACCGTGGCCTACTCTGTCCTGGTGGTCGCCAAGGTCGAGAAGGGCAAGTCTAAG
AAGCTGAAGTCTGTGAAGGAGCTGCTCGGCATCACCATCATGGAGAGAAGCTCTTTCGAGAAGAACCCTATC
GACTTCCTGGAGGCCAAGGGCTACAAGGAGGTGAAGAAGGACCTGATCATCAAGCTGCCCAAGTACTCTCTG
TTCGAGCTGGAGAACGGCCGGAAGAGAATGCTGGCCTCTGCCGGCGAGCTGCAGAAGGGCAACGAGCTGGC
CTTGCCTTCTAAGTACGTGAACTTCTTGTACCTGGCCTCTCACTACGAGAAGCTGAAGGGCTCTCCCGAGGACA
ACGAGCAGAAGCAGCTGTTCGTGGAGCAGCATAAGCATTACCTGGACGAGATCATCGAGCAGATCAGCGAGT
TCTCTAAGCGGGTGATCCTGGCCGACGCCAACCTGGACAAGGTCCTGTCTGCCTACAACAAGCATAGAGACAA
GCCCATCAGAGAGCAGGCCGAGAACATCATCCACCTGTTCACCCTGACCAACCTGGGCGCCCCCGCCGCCTTC
AAGTACTTCGACACCACCATCGACAGAAAGCGGTACACCAGCACCAAGGAGGTGCTCGACGCCACCCTGATC
CATCAGTCTATCACCGGCCTGTACGAGACCAGAATCGACCTGAGCCAGCTGGGGGCGACTGA

The starting Cas9 (old-Cas9) sequence is represented by SEQ ID NO: 2:

(SEQ ID NO: 2)
ATGGACAAGAAGTACAGCATCGGCCTGGACATCGGCACCAACTCTGTGGGCTGGGCCGTGATCACCGACGAG
TACAAGGTGCCCAGCAAGAAATTCAAGGTGCTGGGCAACACCGACCGGCACAGCATCAAGAAGAACCTGATC
GGAGCCCTGCTGTTCGACAGCGGCGAAACAGCCGAGGCCACCCGGCTGAAGAGAACCGCCAGAAGAAGATA
CACCAGACGGAAGAACCGGATCTGCTATCTGCAAGAGATCTTCAGCAACGAGATGGCCAAGGTGGACGACAG
CTTCTTCCACAGACTGGAAGAGTCCTTCCTGGTGGAAGAGGATAAGAAGCACGAGCGGCACCCCATCTTCGG
CAACATCGTGGACGAGGTGGCCTACCACGAGAAGTACCCCACCATCTACCACCTGAGAAAGAAACTGGTGGA
CAGCACCGACAAGGCCGACCTGCGGCTGATCTATCTGGCCCTGGCCCACATGATCAAGTTCCGGGGCCACTTC
CTGATCGAGGGCGACCTGAACCCCGACAACAGCGACGTGGACAAGCTGTTCATCCAGCTGGTGCAGACCTAC
AACCAGCTGTTCGAGGAAAACCCCATCAACGCCAGCGGCGTGGACGCCAAGGCCATCCTGTCTGCCAGACTG
AGCAAGAGCAGACGGCTGGAAAATCTGATCGCCCAGCTGCCCGGCGAGAAGAAGAATGGCCTGTTCGGAAA
CCTGATTGCCCTGAGCCTGGGCCTGACCCCCAACTTCAAGAGCAACTTCGACCTGGCCGAGGATGCCAAACTG
CAGCTGAGCAAGGACACCTACGACGACGACCTGGACAACCTGCTGGCCCAGATCGGCGACCAGTACGCCGAC
CTGTTTCTGGCCGCCAAGAACCTGTCCGACGCCATCCTGCTGAGCGACATCCTGAGAGTGAACACCGAGATCA
CCAAGGCCCCCCTGAGCGCCTCTATGATCAAGAGATACGACGAGCACCACCAGGACCTGACCCTGCTGAAAG
CTCTCGTGCGGCAGCAGCTGCCTGAGAAGTACAAAGAGATTTTCTTCGACCAGAGCAAGAACGGCTACGCCG
GCTACATTGACGGCGGAGCCAGCCAGGAAGAGTTCTACAAGTTCATCAAGCCCATCCTGGAAAAGATGGACG
GCACCGAGGAACTGCTCGTGAAGCTGAACAGAGAGGACCTGCTGCGGAAGCAGCGGACCTTCGACAACGGC
AGCATCCCCCACCAGATCCACCTGGGAGAGCTGCACGCCATTCTGCGGCGGCAGGAAGATTTTTACCCATTCC
TGAAGGACAACCGGGAAAAGATCGAGAAGATCCTGACCTTCCGCATCCCCTACTACGTGGGCCCTCTGGCCA
GGGGAAACAGCAGATTCGCCTGGATGACCAGAAAGAGCGAGGAAACCATCACCCCCTGGAACTTCGAGGAA
GTGGTGGACAAGGGCGCTTCCGCCCAGAGCTTCATCGAGCGGATGACCAACTTCGATAAGAACCTGCCCAAC
GAGAAGGTGCTGCCCAAGCACAGCCTGCTGTACGAGTACTTCACCGTGTATAACGAGCTGACCAAAGTGAAA
TACGTGACCGAGGGAATGAGAAAGCCCGCCTTCCTGAGCGGCGAGCAGAAAAAGGCCATCGTGGACCTGCT
GTTCAAGACCAACCGGAAAGTGACCGTGAAGCAGCTGAAAGAGGACTACTTCAAGAAAATCGAGTGCTTCGA
CTCCGTGGAAATCTCCGGCGTGGAAGATCGGTTCAACGCCTCCCTGGGCACATACCACGATCTGCTGAAAATT
ATCAAGGACAAGGACTTCCTGGACAATGAGGAAAACGAGGACATTCTGGAAGATATCGTGCTGACCCTGACA
CTGTTTGAGGACAGAGAGATGATCGAGGAACGGCTGAAAACCTATGCCCACCTGTTCGACGACAAAGTGATG
AAGCAGCTGAAGCGGCGGAGATACACCGGCTGGGGCAGGCTGAGCCGGAAGCTGATCAACGGCATCCGGG
ACAAGCAGTCCGGCAAGACAATCCTGGATTTCCTGAAGTCCGACGGCTTCGCCAACAGAAACTTCATGCAGCT
GATCCACGACGACAGCCTGACCTTTAAAGAGGACATCCAGAAAGCCCAGGTGTCCGGCCAGGGCGATAGCCT
GCACGAGCACATTGCCAATCTGGCCGGCAGCCCCGCCATTAAGAAGGGCATCCTGCAGACAGTGAAGGTGGT
GGACGAGCTCGTGAAAGTGATGGGCCGGCACAAGCCCGAGAACATCGTGATCGAAATGGCCAGAGAGAACC
AGACCACCCAGAAGGGACAGAAGAACAGCCGCGAGAGAATGAAGCGGATCGAAGAGGGCATCAAAGAGCT
GGGCAGCCAGATCCTGAAAGAACACCCCGTGGAAAACACCCAGCTGCAGAACGAGAAGCTGTACCTGTACTA
CCTGCAGAATGGGCGGGATATGTACGTGGACCAGGAACTGGACATCAACCGGCTGTCCGACTACGATGTGGA
CCATATCGTGCCTCAGAGCTTTCTGAAGGACGACTCCATCGACAACAAGGTGCTGACCAGAAGCGACAAGAA
CCGGGGCAAGAGCGACAACGTGCCCTCCGAAGAGGTCGTGAAGAAGATGAAGAACTACTGGCGGCAGCTGC
TGAACGCCAAGCTGATTACCCAGAGAAAGTTCGACAATCTGACCAAGGCCGAGAGAGGCGGCCTGAGCGAA
CTGGATAAGGCCGGCTTCATCAAGAGACAGCTGGTGGAAACCCGGCAGATCACAAAGCACGTGGCACAGATC
CTGGACTCCCGGATGAACACTAAGTACGACGAGAATGACAAGCTGATCCGGGAAGTGAAAGTGATCACCCTG
AAGTCCAAGCTGGTGTCCGATTTCCGGAAGGATTTCCAGTTTTACAAAGTGCGCGAGATCAACAACTACCACC
ACGCCCACGACGCCTACCTGAACGCCGTCGTGGGAACCGCCCTGATCAAAAAGTACCCTAAGCTGGAAAGCG
AGTTCGTGTACGGCGACTACAAGGTGTACGACGTGCGGAAGATGATCGCCAAGAGCGAGCAGGAAATCGGC
AAGGCTACCGCCAAGTACTTCTTCTACAGCAACATCATGAACTTTTTCAAGACCGAGATTACCCTGGCCAACGG
CGAGATCCGGAAGCGGCCTCTGATCGAGACAAACGGCGAAACCGGGGAGATCGTGTGGGATAAGGGCCGG
GATTTTGCCACCGTGCGGAAAGTGCTGAGCATGCCCCAAGTGAATATCGTGAAAAAGACCGAGGTGCAGACA
GGCGGCTTCAGCAAAGAGTCTATCCTGCCCAAGAGGAACAGCGATAAGCTGATCGCCAGAAAGAAGGACTG
GGACCCTAAGAAGTACGGCGGCTTCGACAGCCCCACCGTGGCCTATTCTGTGCTGGTGGTGGCCAAAGTGGA
AAAGGGCAAGTCCAAGAAACTGAAGAGTGTGAAAGAGCTaCTGGGGATCACCATCATGGAAAGAAGCAGCT
TCGAGAAGAATCCCATCGACTTTCTGGAAGCCAAGGGCTACAAAGAAGTGAAAAAGGACCTGATCATCAAGC
TGCCTAAGTACTCCCTGTTCGAGCTGGAAAACGGCCGGAAGAGAATGCTGGCCTCTGCCGGCGAACTGCAGA
AGGGAAACGAACTGGCCCTGCCCTCCAAATATGTGAACTTCCTGTACCTGGCCAGCCACTATGAGAAGCTGAA
GGGCTCCCCCGAGGATAATGAGCAGAAACAGCTGTTTGTGGAACAGCACAAGCACTACCTGGACGAGATCAT
CGAGCAGATCAGCGAGTTCTCCAAGAGAGTGATCCTGGCCGACGCTAATCTGGACAAAGTGCTGTCCGCCTA
CAACAAGCACCGGGATAAGCCCATCAGAGAGCAGGCCGAGAATATCATCCACCTGTTTACCCTGACCAATCTG
GGAGCCCCTGCCGCCTTCAAGTACTTTGACACCACCATCGACCGGAAGAGGTACACCAGCACCAAAGAGGTG
CTGGACGCCACCCTGATCCACCAGAGCATCACCGGCCTGTACGAGACACGGATCGACCTGTCTCAGCTGGGA
GGCGACTAG

Old-Cas9 is a commercially available Cas9 sequence that has been codon optimised using human generic codon usage. To codon optimise this sequence using tubulin III codon usage frequencies, the inventors replaced 33% of codons with synonymous codons that are preferred by tubulin III (463 codons of the original Cas9 sequence were replaced). For example, 100% of the alanine, glutamine, lysine and tyrosine codons in CodOpt-Cas9 are provided by the tubulin III preferred codons GCC, CAG, AAG and TAC, respectively (these codons are used at 93%, 96%, 77% and 87% frequency, respectively, in Old-Cas9). In addition, Old-Cas9 codons that are not used by tubulin III were replaced with tubulin III preferred codons, e.g. lysine AAA codons were replaced with AAG and tyrosine TAT codons were replaced with TAC.

To test whether CodOpt-Cas9 could be expressed, both Old-Cas9 and CodOpt-Cas9 were expressed in human embryonic kidney 293 (HEK293) cells. Surprisingly, CodOpt-Cas9 exhibited higher expression than old-Cas9 at both the mRNA and protein levels in HEK293 cells. These high levels of Cas9 contributed to CodOpt-Cas9 HEK293 cells demonstrating higher nuclease activity and faster cutting efficiency compared to HEK293 cells containing old-Cas9. However, it should be noted that Tubulin III is not highly expressed in HEK293 cells, and so these results suggest that tubulin III's codon biases are not unique to neurons.

The inventors then tested whether CodOpt-Cas9 could be readily expressed in iPSCs. Similar to results in HEK293 cells, CodOpt-Cas9 achieved higher expression levels in iPSCs than old-Cas9. As mentioned previously, old-Cas9 expression drops dramatically during differentiation of iPSCs to neuronal cell types and so the inventors next sought to differentiate iPSCs expression CodOpt-Cas9.

Advantageously, the inventors discovered that CodOpt-Cas9 was expressed throughout differentiation of iPSCs and that CodOpt-Cas9 remained detectable in differentiated neuronal cells, whereas old-Cas9 showed a sharp decrease in expression levels and ultimately became undetectable as cells entered a more neuronal phenotype. These results confirm that codon optimising Cas9 based on the codon biases of tubulin III achieves efficient and sustained expression of Cas9 in iPSC derived differentiated neurons.

To test whether the advantageous results described above are limited to iPSC derived neuronal cells, the inventors attempted to express CodOpt-Cas9 in iPSC derived hepatocytes (which do not typically express tubulin III). Similar to iPSC derived neuronal cells, old-Cas9 exhibits a sharp decrease in expression levels during differentiation of hepatocytes, whereas CodOpt-Cas9 achieved and maintained significantly higher expression levels throughout differentiation. Advantageously, together with the increased expression observed in HEK293 cells, these results demonstrate that codon optimising a sequence based on the codon biases of tubulin III achieves increased and sustained expression in numerous different cells types, including those that do not typically express tubulin III.

The inventors next sought to determine whether expression of Cas9 could be ‘tuned’ through partial codon optimisation. A Cas9 variant was generated wherein the first 606 N-terminal amino acids were codon optimised using tubulin III preferred codons while the rest of the sequence remained unaltered. This N-terminal codon optimised Cas9 variant, referred to herein as NOpt-Cas9, is represented by SEQ ID NO: 3:

(SEQ ID NO: 3)
ATGGACAAGAAGTACTCTATCGGCCTGGACATCGGCACCAACAGCGTGGGCTGGGCCGTCATCACCGACGAG
TACAAGGTGCCTTCTAAGAAGTTCAAGGTGCTGGGCAACACCGACCGCCATTCTATCAAGAAGAACCTGATCG
GCGCCCTGCTGTTCGACTCTGGCGAGACCGCCGAGGCCACCAGACTGAAGCGGACCGCCCGACGCCGATACA
CCAGACGGAAGAACAGAATCTGCTACCTTCAGGAGATCTTCAGCAACGAGATGGCCAAGGTGGACGACTCTT
TCTTCCATCGCCTGGAGGAGAGCTTCCTGGTGGAGGAGGACAAGAAGCATGAGCGCCATCCTATCTTCGGCA
ACATCGTGGACGAGGTGGCCTACCATGAGAAGTACCCTACCATCTACCATCTGAGGAAGAAGCTGGTGGACT
CTACGGACAAGGCCGACCTGAGACTTATCTACCTGGCCCTGGCCCATATGATCAAGTTCCGGGGCCATTTCCTC
ATCGAGGGCGACCTCAACCCCGACAACAGCGACGTGGACAAGCTGTTCATCCAGTTGGTGCAGACCTACAAC
CAGCTTTTCGAGGAGAACCCCATCAACGCCTCTGGCGTGGACGCCAAGGCCATCCTGAGTGCCCGCCTGTCTA
AGAGCCGCAGACTTGAGAACCTGATCGCCCAGCTGCCCGGCGAGAAGAAGAACGGCCTGTTCGGCAACCTTA
TCGCCCTGTCTCTGGGCCTTACCCCTAACTTCAAGTCTAACTTCGACCTGGCCGAGGACGCCAAGCTGCAGCTT
AGCAAGGACACCTACGACGACGACTTGGACAACCTGCTTGCCCAGATCGGCGACCAGTACGCCGACCTGTTCC
TGGCCGCCAAGAACTTGAGCGACGCCATCCTGCTTAGCGACATCCTGAGAGTCAACACCGAGATCACCAAGG
CCCCTCTGTCTGCCAGCATGATCAAGCGGTACGACGAGCATCACCAGGACCTGACCCTGTTGAAGGCCCTCGT
GCGACAGCAGCTGCCTGAGAAGTACAAGGAGATCTTCTTTGACCAGAGCAAGAACGGCTACGCCGGCTACAT
CGACGGCGGCGCCTCTCAGGAGGAGTTCTACAAGTTCATCAAGCCCATCCTGGAGAAGATGGACGGCACCGA
GGAGCTTCTGGTCAAGCTGAACAGGGAGGACCTGCTTAGGAAGCAGCGCACCTTCGACAACGGCTCAATCCC
TCATCAGATCCACCTGGGCGAGTTGCATGCCATCCTCAGACGCCAGGAGGACTTCTACCCCTTCCTGAAGGAC
AACAGGGAGAAGATCGAGAAGATCCTGACCTTCCGAATCCCCTACTACGTGGGCCCTCTGGCCCGAGGCAAC
TCTCGATTCGCCTGGATGACCCGCAAGTCTGAGGAGACCATCACCCCTTGGAACTTCGAGGAGGTCGTGGACA
AGGGCGCCTCTGCCCAGTCATTCATCGAGCGGATGACCAACTTCGACAAGAACCTGCCCAACGAGAAGGTGC
TGCCTAAGCATTCTTTGCTGTACGAGTACTTCACCGTGTACAACGAGCTGACCAAGGTGAAGTACGTGACCGA
GGGCATGCGCAAGCCTGCCTTCCTGTCTGGCGAGCAGAAGAAGGCCATCGTGGACCTGTTGTTCAAGACCAA
CCGGAAGGTGACCGTGAAGCAGCTGAAGGAGGACTACTTCAAGAAGATCGAGTGCTTCGACTCTGTGGAGAT
CAGCGGCGTGGAGGACCGCTTCAACGCCTCTCTGGGCACATACCACGATCTGCTGAAAATTATCAAGGACAA
GGACTTCCTGGACAATGAGGAAAACGAGGACATTCTGGAAGATATCGTGCTGACCCTGACACTGTTTGAGGA
CAGAGAGATGATCGAGGAACGGCTGAAAACCTATGCCCACCTGTTCGACGACAAAGTGATGAAGCAGCTGAA
GCGGCGGAGATACACCGGCTGGGGCAGGCTGAGCCGGAAGCTGATCAACGGCATCCGGGACAAGCAGTCCG
GCAAGACAATCCTGGATTTCCTGAAGTCCGACGGCTTCGCCAACAGAAACTTCATGCAGCTGATCCACGACGA
CAGCCTGACCTTTAAAGAGGACATCCAGAAAGCCCAGGTGTCCGGCCAGGGCGATAGCCTGCACGAGCACAT
TGCCAATCTGGCCGGCAGCCCCGCCATTAAGAAGGGCATCCTGCAGACAGTGAAGGTGGTGGACGAGCTCGT
GAAAGTGATGGGCCGGCACAAGCCCGAGAACATCGTGATCGAAATGGCCAGAGAGAACCAGACCACCCAGA
AGGGACAGAAGAACAGCCGCGAGAGAATGAAGCGGATCGAAGAGGGCATCAAAGAGCTGGGCAGCCAGAT
CCTGAAAGAACACCCCGTGGAAAACACCCAGCTGCAGAACGAGAAGCTGTACCTGTACTACCTGCAGAATGG
GCGGGATATGTACGTGGACCAGGAACTGGACATCAACCGGCTGTCCGACTACGATGTGGACCATATCGTGCC
TCAGAGCTTTCTGAAGGACGACTCCATCGACAACAAGGTGCTGACCAGAAGCGACAAGAACCGGGGCAAGA
GCGACAACGTGCCCTCCGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAGCGAACTGGATAAGGC
CGGCTTCATCAAGAGACAGCTGGTGGAAACCCGGCAGATCACAAAGCACGTGGCACAGATCCTGGACTCCCG
GATGAACACTAAGTACGACGAGAATGACAAGCTGATCCGGGAAGTGAAAGTGATCACCCTGAAGTCCAAGCT
GGTGTCCGATTTCCGGAAGGATTTCCAGTTTTACAAAGTGCGCGAGATCAACAACTACCACCACGCCCACGAC
GCCTACCTGAACGCCGTCGTGGGAACCGCCCTGATCAAAAAGTACCCTAAGCTGGAAAGCGAGTTCGTGTAC
GGCGACTACAAGGTGTACGACGTGCGGAAGATGATCGCCAAGAGCGAGCAGGAAATCGGCAAGGCTACCGC
CAAGTACTTCTTCTACAGCAACATCATGAACTTTTTCAAGACCGAGATTACCCTGGCCAACGGCGAGATCCGG
AAGCGGCCTCTGATCGAGACAAACGGCGAAACCGGGGAGATCGTGTGGGATAAGGGCCGGGATTTTGCCAC
CGTGCGGAAAGTGCTGAGCATGCCCCAAGTGAATATCGTGAAAAAGACCGAGGTGCAGACAGGCGGCTTCA
GCAAAGAGTCTATCCTGCCCAAGAGGAACAGCGATAAGCTGATCGCCAGAAAGAAGGACTGGGACCCTAAG
AAGTACGGCGGCTTCGACAGCCCCACCGTGGCCTATTCTGTGCTGGTGGTGGCCAAAGTGGAAAAGGGCAAG
TCCAAGAAACTGAAGAGTGTGAAAGAGCTaCTGGGGATCACCATCATGGAAAGAAGCAGCTTCGAGAAGAAT
CCCATCGACTTTCTGGAAGCCAAGGGCTACAAAGAAGTGAAAAAGGACCTGATCATCAAGCTGCCTAAGTACT
CCCTGTTCGAGCTGGAAAACGGCCGGAAGAGAATGCTGGCCTCTGCCGGCGAACTGCAGAAGGGAAACGAA
CTGGCCCTGCCCTCCAAATATGTGAACTTCCTGTACCTGGCCAGCCACTATGAGAAGCTGAAGGGCTCCCCCG
AGGATAATGAGCAGAAACAGCTGTTTGTGGAACAGCACAAGCACTACCTGGACGAGATCATCGAGCAGATCA
GCGAGTTCTCCAAGAGAGTGATCCTGGCCGACGCTAATCTGGACAAAGTGCTGTCCGCCTACAACAAGCACC
GGGATAAGCCCATCAGAGAGCAGGCCGAGAATATCATCCACCTGTTTACCCTGACCAATCTGGGAGCCCCTGC
CGCCTTCAAGTACTTTGACACCACCATCGACCGGAAGAGGTACACCAGCACCAAAGAGGTGCTGGACGCCAC
CCTGATCCACCAGAGCATCACCGGCCTGTACGAGACACGGATCGACCTGTCTCAGCTGGGAGGCGACTGA

Similar to CodOpt-Cas9, NOpt-Cas9 exhibited improved cutting efficiency relative to old-Cas9 as iPSC cells progressed to a more neuronal cell type. In the later stages of differentiation, e.g. days 10 and 14, NOpt-Cas9 demonstrated reduced expression and therefore reduced cutting efficiency relative to CodOpt-Cas9 suggesting that the degree of codon optimisation directly impacts the level of protein production as neurons mature. Advantageously, these results indicate that it is not necessary to codon optimise the full Cas9 nucleic acid sequence to achieve increased expression, and Cas9 activity can be tuned by adjusting the level of codon optimisation with fully codon optimised Cas9 exhibiting higher activity than partially codon optimised variants.

The results described herein demonstrate that target nucleic acid sequences (e.g. genes of interest) that are codon optimised based on the codon biases exhibited by an endogenous gene encoding a protein which is highly expressed by the host cell, or based on the codon biases exhibited by an endogenous gene encoding a protein that is highly expressed in a cell from the same species as the host cell, achieve higher level and more sustained expression. Advantageously, sequences that are codon optimised according to the invention achieve higher expression than sequences that are codon optimised using current gold standard methods which typically rely on species level codon biases. In addition, the inventors have shown that gene expression can be adjusted by altering the degree to which sequences are codon optimised using the methods described herein.

The invention provides a method for codon optimising a target nucleic acid sequence for expression in a host cell comprising altering the codon usage frequency of the target nucleic acid sequence based on the codon usage frequency of a gene encoding a protein that is highly expressed in the host cell or the codon usage frequency of a gene encoding a protein that is highly expressed in a cell from the same species as the host cell. In some embodiments, the invention provides a method for codon optimising the target nucleic acid sequence for improved expression in the host cell. In some embodiments, the invention provides a method for codon optimising the target nucleic acid sequence for increased expression in the host cell. In some embodiments, the gene encoding a protein that is highly expressed in the host cell is an endogenous gene. In some embodiments, the gene encoding a protein that is highly expressed in a cell from the same species as the host cell is an endogenous gene.

As used herein “codon usage frequency” (also referred to herein as “codon frequency” or “usage frequency”) refers to the proportion of each synonymous codon (each codon encoding the same amino acid) that is present in a sequence or group of sequences. A codon usage frequency of 100% indicates exclusive use of the codon in question for a given amino acid. Methionine (met) and tryptophan (trp) are each encoded by a single codon and so these codons always have a usage frequency of 100%. A codon usage frequency of 0% indicates that the codon is not used by the sequence/group of sequences. A codon usage frequency of 25% for a given codon indicates that the codon in question accounts for 25% of all of the synonymous codons present in the sequence/group of sequences that encode the encoded amino acid (with the other synonymous codon(s) accounting for the remaining 75%). In some embodiments, the method comprises determining the codon usage frequency of the gene encoding a protein that is highly expressed by the host cell, or the codon usage frequency of a gene encoding a protein that is highly expressed by a cell from the same species as the host cell.

Hereinafter, a “gene encoding a highly expressed protein” refers to a gene encoding a protein that is highly expressed in the host cell or in a cell from the same species as the host cell.

In some embodiments, a non-preferred codon is a codon that is used with lower frequency by the gene encoding a highly expressed protein than would be expected if each synonymous codon was used at random. Random usage frequency depends on the number of synonymous codons available for a given amino acid. For example, for an amino acid that is encoded by two synonymous codons, each of these synonymous codons would have a random usage frequency of 50%. In this scenario, a codon usage frequency of less than 50% indicates that a codon is non-preferred. Similarly, for an amino acid that is encoded by six synonymous codons, each of these synonymous codons would have a random usage frequency of 16.67%, and a codon usage frequency of less than 16.67% indicates that a codon is non-preferred. In some embodiments, a non-preferred codon is a codon that is used with lower frequency by the gene encoding a highly expressed protein than other synonymous codon(s) encoding the same amino acid.

In some embodiments, a non-preferred codon is a codon that is used by the gene encoding a highly expressed protein with a frequency of less than 50%, less than 45%, less than 40%, less than 35%, less than 33%, less than 30%, less than 25%, less than 20%, less than 16%, less than 15%, less than 10%, less than 5%, or 0%. In some embodiments, non-preferred codons are used with less than 10% frequency by the gene encoding the highly expressed protein. In some embodiments, non-preferred codons are used with 0% frequency by the gene encoding the highly expressed protein.

In some embodiments, a preferred codon refers to a codon that is used with higher frequency by the gene encoding a highly expressed protein than would be expected if each synonymous codon was used at random. As mentioned above, random usage frequency depends on the number of synonymous codons available for a given amino acid. For example, for an amino acid that is encoded by two synonymous codons, each of these codons would have a random usage frequency of 50%, and so a codon usage frequency of more than 50% indicates a preference for that codon. For an amino acid that is encoded by six synonymous codons, each of these synonymous codon would have a random usage frequency of 16.67% and so a codon usage frequency of more than 16.67% indicates that a codon is preferred. In some embodiments, a preferred codon is a codon that is used with higher frequency by the gene encoding a highly expressed protein than other synonymous codon(s) encoding the same amino acid. In some embodiments, a preferred codon is a codon that is used exclusively by the gene encoding a highly expressed protein.

In some embodiments, a preferred codon is a codon that is used by the gene encoding a highly expressed protein with a frequency of at least 17%, at least 20%, at least 25%, at least 30%, at least 34%, at least 35%, at least 40%, at least 45%, at least 50%, at least 55%, at least 60%, at least 65%, at least 70%, at least 75%, at least 80%, at least 85%, at least 90%, at least 95%, or 100%. In some embodiments, preferred codons are used with at least 50% frequency by the gene encoding the highly expressed protein. In some embodiments, preferred codons are used with at least 75% frequency by the gene encoding the highly expressed protein.

In some embodiments, at least 50% of non-preferred codons within the target nucleic acid sequence are replaced with preferred synonymous codons. In some embodiments, the method comprises replacing at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of non-preferred codons within the target nucleic acid sequence with preferred synonymous codons. In some embodiments, the method comprises replacing all non-preferred codons within the target nucleic acid sequence that are used by the gene encoding the highly expressed protein with a frequency of 0% with preferred synonymous codons.

In some embodiments, the method comprises replacing all non-preferred codons within the target nucleic acid sequence with preferred synonymous codons in a specific region of the target nucleic acid, e.g. the 5′ end of the target nucleic acid (encoding the N-terminal region of the protein). In some embodiments, the method comprises replacing all non-preferred codons with preferred synonymous codons in the first at least 100, at least 200, at least 300, at least 400, at least 500, at least 600, at least 700, at least 800, at least 900, at least 1000, at least 1500 or at least 2000 codons starting from the 5′ end of the target nucleic acid.

As used herein, a highly expressed protein is a protein that is expressed constitutively by the host cell or by a related cell from the same species as the host cell. Typically, a highly expressed protein can be readily detected using methods known in the art, e.g. Western blotting and enzyme-linked immunosorbent assay (ELISA). Preferably, the highly expressed protein is one of the most highly and/or stably expressed proteins produced by the host cell or the related cell. In some embodiments, the highly expressed protein is among the top 10% most highly expressed proteins within the host cell or the cell from the same species as the host cell. The skilled person can readily identify highly expressed proteins using methods known in the art, e.g. proteomic approaches including gel electrophoresis and mass spectrometry. Highly expressed proteins can also be identified using an online protein expression database, e.g. the human protein atlas.

In some embodiments, the highly expressed protein is a housekeeping protein or a marker protein. As used herein, a “housekeeping protein” is a constitutively expressed protein that is required for the maintenance of basic cellular function in the host cell or cell from the same species as the host cell, e.g. in humans, GAPDH, β-tubulin and β-actin are considered housekeeping genes. In some embodiments, the gene encoding the highly expressed protein is the GAPDH gene. In some embodiments, the gene encoding the highly expressed protein is the β-actin gene. In some embodiments, the gene encoding the highly expressed protein is the β-tubulin gene. As used herein, a “cell marker protein” is a protein that is expressed by a particular cell type that can be used to identify that cell type, e.g. tubulin III (also referred to as β-tubulin III, class III β-tubulin or βIII-tubulin) which is a neuronal cell marker, myosin which is a muscle cell marker, and alpha-fetoprotein which is a hepatic stem cell marker. In some embodiments, the gene encoding the highly expressed protein is the tubulin III gene. In some embodiments, the gene encoding the highly expressed protein is a tubulin III gene transcript, e.g. the tubulin III transcript represented by SEQ ID NO: 8. In some embodiments, the gene encoding the highly expressed protein is the myosin gene. In some embodiments, the gene encoding the highly expressed protein is the alpha-fetoprotein gene. The highly expressed protein may be a lymphocyte marker protein, e.g. a T cell marker protein such as CD4. In some embodiments, the gene encoding the highly expressed protein is the CD4 gene. In some embodiments, non-preferred codons comprise codons that are used by the gene encoding the highly expressed protein with a frequency of 0%. For example, when the gene encoding the highly expressed protein is the tubulin III gene, non-preferred codons may include: alanine codons GCA, GCG and GCT; arginine codons AGA and CGT; cysteine codon TGT; glutamine codon CAA; isoleucine codon ATA; leucine codons CTA and TTA; lysine codon AAA; proline codon CCG; serine codon TCC; threonine codons ACA, ACG and ACT; tyrosine codon TAT; valine codons GTA and GTT; and stop or end codons TAA and TAG. In some embodiments, non-preferred codons comprise codons that are used by the gene encoding the highly expressed protein with lower frequency than would be expected if each synonymous codon was used at random. For example, when the gene encoding the highly expressed protein is the tubulin III gene, non-preferred codons may also include: asparagine codon AAT; aspartic acid codon GAT; glutamic acid codon GAA; glycine codons GGA, GGG and GGT; histidine codon CAC; isoleucine codon ATT; leucine codons CTC, CTT and TTG; phenylalanine codon TTT; proline codon CCA; serine codons TCA and TCG; and valine codon GTC.

In some embodiments, preferred codons comprise codons that are used by the gene encoding the highly expressed protein with a frequency of 100%. For example, when the gene encoding the highly expressed protein is the tubulin III gene, preferred codons may include: alanine codon GCC; cysteine codon TGC; glutamine codon CAG; lysine codon AAG; threonine codon ACC; tyrosine codon TAC; and the stop codon TGA. In some embodiments, preferred codons comprise codons that are used with higher frequency by the gene encoding the highly expressed protein than other synonymous codon(s) encoding the same amino acid. For example, when the gene is tubulin III gene, preferred codons may also include: arginine codons AGG, CGA, CGC and CGG; asparagine codon AAC; aspartic acid codon GAC; glutamic acid codon GAG; glycine codon GGC; histidine codon CAT; isoleucine codon ATC; leucine codon CTG; phenylalanine codon TTC; proline codons CCC and CCT; serine codons AGC, AGT and TCT; and valine codon GTG.

In some embodiments, the host cell is a human cell. In some embodiments, the host cell is an iPSC cell, or a differentiated cell derived from an iPSC. In some embodiments, the host cell is an iPSC derived neuron. In some embodiments, the host cell is a cortical neuron, dopaminergic neuron or a motor neuron. In some embodiments, the host cell is an iPSC derived macrophage. In some embodiments, the host cell is an iPSC derived cardiomyocytes. In some embodiments, the host cell is an iPSC derived hepatocyte. In some embodiments, the host cell is a HEK293 cell. For each of these embodiments, in some embodiments, the gene encoding the highly expressed protein is the tubulin III gene.

In some embodiments, the host cell is a bacterial cell. In some embodiments, the host cell is selected from Escherichia coli, Pseudomonas (e.g. P. aeruginosa, P. putida, P. fluorescens), Lactobacillus (e.g. L. lactis), Streptomyces (e.g. S. coelicolor), Bacillus (e.g. B. subtilis), Acinetobacter, Agrobacterium, Cupriavidus, Clostridium, Rhodobacter, Marinobacter, Klebsiella, Ralstonia, and Rhodococcus.

In some embodiments, the host cell is a yeast cell. In some embodiments, the host cell is selected from Saccharomyces (e.g. S. cerevisiae), Schizosaccharomyces (e.g. S. pombe), Candida (e.g. C. albicans), Pichia, Hansenula, Klockera, Schwanniomyces, Rhodosporidium, Yarrowia and Rhodotorula.

In some embodiments, the host cell is a fungal cell. In some embodiments, the host cell is selected from Aspergillus (e.g. A. niger), Penicillium, Rhizopus, Chrysosporium, Myceliophthora, Trichoderma (e.g. T. reesei), Humicola, Acremonium and Fusarium.

In some embodiments, the target nucleic acid is a heterologous nucleic acid. In some embodiments, the target nucleic acid is an endogenous nucleic acid.

In some embodiments, the target nucleic acid encodes a Cas enzyme. In some embodiments, the target nucleic acid encodes Cas9. In some embodiments, the target nucleic acid encodes Cas12a. In some embodiments, the target nucleic acid encodes Cas13Rx.

The invention provides a nucleic acid sequence that has been codon optimised by the method of the invention. In some embodiments, the invention provides a codon optimised nucleic acid encoding Cas9. In some embodiments, the codon optimised nucleic acid encoding Cas9 comprises at least 70%, at least 75%, at least 80%, at least 85%, at least 90%, at least 95% or 100% sequence identity to SEQ ID NO: 1.

In some embodiments, the invention provides a nucleic acid encoding Cas9 wherein the 5′ region of the nucleic acid is codon optimised by the method of the invention. In some embodiments, the nucleic acid comprises at least 70%, at least 75%, at least 80%, at least 85%, at least 90%, at least 95% or 100% sequence identity to SEQ ID NO: 3.

In some embodiments, the invention provides a codon optimised nucleic acid encoding Cas12a. In some embodiments, the codon optimised nucleic acid encoding Cas12a comprises at least 70%, at least 75%, at least 80%, at least 85%, at least 90%, at least 95% or 100% sequence identity to SEQ ID NO: 4.

In some embodiments, the invention provides a codon optimised nucleic acid encoding Cas13Rx. In some embodiments, the codon optimised nucleic acid encoding Cas13Rx comprises at least 70%, at least 75%, at least 80%, at least 85%, at least 90%, at least 95% or 100% sequence identity to SEQ ID NO: 5.

The invention also provides a vector comprising a nucleic acid that has been codon optimised by a method of the invention. In some embodiments, the vector comprises a nucleic acid of the invention.

Suitable vectors will depend on the host cell used, and can be readily identified by the skilled person. In some embodiments, the vector is selected from an adeno-associated virus (AAV) vector, a HIV-based lentivirus vector, equine immunodeficiency virus (EIV) vector, a feline immunodeficiency virus (FIV) vector, and a herpes simplex virus vector.

A vector may comprise one or more of an origin of replication, a promoter sequence operably linked to a nucleic acid of the invention and a reporter gene or selectable marker. The promoter may be homologous or heterologous. The promoter may be constitutive or inducible. In some embodiments, the promoter is inducible and is activated in the presence of an inducing agent. Inducing agents include, but are not limited to, sugars, metal salts, and antibiotics. Typically, the promoter is operable in the host cell of interest.

In some embodiments, the vector comprises a codon optimised nucleic acid encoding a Cas enzyme. In some embodiments, the vector comprises a codon optimised nucleic acid encoding Cas9, Cas12a or Cas13Rx. In some embodiments, the vector comprises a nucleic acid having at least at least 70%, at least 75%, at least 80%, at least 85%, at least 90%, at least 95% or 100% sequence identity to any of SEQ ID NOs: 1, 3, 4 or 5.

The invention also provides a host cell comprising a nucleic acid sequence that has been codon optimised by a method of the invention. In some embodiments, the host cell comprises a nucleic acid of the invention. In some embodiments, the host cell comprises a vector of the invention.

In some embodiments, the host cell is a human cell. In some embodiments, the host cell is an iPSC cell, or a differentiated cell derived from iPSCs. In some embodiments, the host cell is an iPSC derived neuron. In some embodiments, the host cell is an iPSC derived macrophage. In some embodiments, the host cell is an iPSC derived cardiomyocytes. In some embodiments, the host cell is an iPSC derived hepatocyte. In some embodiments, the host cell is a HEK293 cell. In

In some embodiments, the host cell is a bacterial cell. In some embodiments, the host cell is selected from Escherichia coli, Pseudomonas (e.g. P. aeruginosa, P. putida, P. fluorescens), Lactobacillus (e.g. L. lactis), Streptomyces (e.g. S. coelicolor), Bacillus (e.g. B. subtilis), Acinetobacter, Agrobacterium, Cupriavidus, Clostridium, Rhodobacter, Marinobacter, Klebsiella, Ralstonia, and Rhodococcus. In some embodiments, the host cell is a yeast cell. In some embodiments, the host cell is selected from Saccharomyces (e.g. S. cerevisiae), Schizosaccharomyces (e.g. S. pombe), Candida (e.g. C. albicans),

Pichia, Hansenula, Klockera, Schwanniomyces, Rhodosporidium, Yarrowia and Rhodotorula. In some embodiments, the host cell is a fungal cell. In some embodiments, the host cell is selected from Aspergillus (e.g. A. niger), Penicillium, Rhizopus, Chrysosporium, Myceliophthora, Trichoderma (e.g. T. reesei), Humicola, Acremonium and Fusarium.

In some embodiments, the host cell comprises a codon optimised nucleic acid encoding a Cas enzyme. In some embodiments, the host cell comprises a codon optimised nucleic acid encoding Cas9, Cas12a or Cas13Rx. In some embodiments, the host cell comprises a nucleic acid having at least 70%, at least 75%, at least 80%, at least 85%, at least 90%, at least 95% or 100% sequence identity to any of SEQ ID NOs: 1, 3, 4 or 5.

The invention provides an iPSC comprising a nucleic acid sequence encoding Cas9, wherein the nucleic acid sequence has been codon optimised by the method of the invention. In some embodiments, the iPSC comprises a nucleic acid having at least 70%, at least 75%, at least 80%, at least 85%, at least 90%, at least 95% or 100% sequence identity to any of SEQ ID NO: 1.

The invention provides a neuronal cell derived from an iPSC, wherein the neuronal cell comprises a nucleic acid sequence encoding Cas9, wherein the nucleic acid sequence has been codon optimised by the method of the invention. In some embodiments, the neuronal cell derived from an iPSC comprises a nucleic acid having at least 70%, at least 75%, at least 80%, at least 85%, at least 90%, at least 95% or 100% sequence identity to any of SEQ ID NO: 1.

The invention provides a hepatocyte derived from an iPSC, wherein the hepatocyte comprises a nucleic acid sequence encoding Cas9, wherein the nucleic acid sequence has been codon optimised by the method of the invention. In some embodiments, the hepatocyte derived from an iPSC comprises a nucleic acid having at least 70%, at least 75%, at least 80%, at least 85%, at least 90%, at least 95% or 100% sequence identity to any of SEQ ID NO: 1.

The invention provides a macrophage derived from an iPSC, wherein the macrophage comprises a nucleic acid sequence encoding Cas9, wherein the nucleic acid sequence has been codon optimised by the method of the invention. In some embodiments, the macrophage derived from an iPSC comprises a nucleic acid having at least 70%, at least 75%, at least 80%, at least 85%, at least 90%, at least 95% or 100% sequence identity to any of SEQ ID NO: 1.

The invention provides a cardiomyocyte derived from an iPSC, wherein the cardiomyocyte comprises a nucleic acid sequence encoding Cas9, wherein the nucleic acid sequence has been codon optimised by the method of the invention. In some embodiments, the cardiomyocyte derived from an iPSC comprises a nucleic acid having at least 70%, at least 75%, at least 80%, at least 85%, at least 90%, at least 95% or 100% sequence identity to any of SEQ ID NO: 1.

The invention provides an iPSC comprising a nucleic acid sequence encoding Cas9, wherein the nucleic acid sequence has been codon optimised by the method of the invention. In some embodiments, the iPSC comprises a nucleic acid having at least 70%, at least 75%, at least 80%, at least 85%, at least 90%, at least 95% or 100% sequence identity to any of SEQ ID NO: 3.

The invention provides a neuronal cell derived from an iPSC, wherein the neuronal cell comprises a nucleic acid sequence encoding Cas9, wherein the nucleic acid sequence has been codon optimised by the method of the invention. In some embodiments, the neuronal cell derived from an iPSC comprises a nucleic acid having at least 70%, at least 75%, at least 80%, at least 85%, at least 90%, at least 95% or 100% sequence identity to any of SEQ ID NO: 3.

The invention provides a hepatocyte derived from an iPSC, wherein the hepatocyte comprises a nucleic acid sequence encoding Cas9, wherein the nucleic acid sequence has been codon optimised by the method of the invention. In some embodiments, the hepatocyte derived from an iPSC comprises a nucleic acid having at least 70%, at least 75%, at least 80%, at least 85%, at least 90%, at least 95% or 100% sequence identity to any of SEQ ID NO: 3.

The invention provides a macrophage derived from an iPSC, wherein the macrophage comprises a nucleic acid sequence encoding Cas9, wherein the nucleic acid sequence has been codon optimised by the method of the invention. In some embodiments, the macrophage derived from an iPSC comprises a nucleic acid having at least 70%, at least 75%, at least 80%, at least 85%, at least 90%, at least 95% or 100% sequence identity to any of SEQ ID NO: 3.

The invention provides a cardiomyocyte derived from an iPSC, wherein the cardiomyocyte comprises a nucleic acid sequence encoding Cas9, wherein the nucleic acid sequence has been codon optimised by the method of the invention. In some embodiments, the cardiomyocyte derived from an iPSC comprises a nucleic acid having at least 70%, at least 75%, at least 80%, at least 85%, at least 90%, at least 95% or 100% sequence identity to any of SEQ ID NO: 3.

The invention provides an iPSC comprising a nucleic acid sequence encoding Cas12a, wherein the nucleic acid sequence has been codon optimised by the method of the invention. In some embodiments, the iPSC comprises a nucleic acid having at least 70%, at least 75%, at least 80%, at least 85%, at least 90%, at least 95% or 100% sequence identity to any of SEQ ID NO: 4.

The invention provides a neuronal cell derived from an iPSC, wherein the neuronal cell comprises a nucleic acid sequence encoding Cas12a, wherein the nucleic acid sequence has been codon optimised by the method of the invention. In some embodiments, the neuronal cell derived from an iPSC comprises a nucleic acid having at least 70%, at least 75%, at least 80%, at least 85%, at least 90%, at least 95% or 100% sequence identity to any of SEQ ID NO: 4.

The invention provides a hepatocyte derived from an iPSC, wherein the hepatocyte comprises a nucleic acid sequence encoding Cas12a, wherein the nucleic acid sequence has been codon optimised by the method of the invention. In some embodiments, the hepatocyte derived from an iPSC comprises a nucleic acid having at least 70%, at least 75%, at least 80%, at least 85%, at least 90%, at least 95% or 100% sequence identity to any of SEQ ID NO: 4.

The invention provides a macrophage derived from an iPSC, wherein the macrophage comprises a nucleic acid sequence encoding Cas12a, wherein the nucleic acid sequence has been codon optimised by the method of the invention. In some embodiments, the macrophage derived from an iPSC comprises a nucleic acid having at least 70%, at least 75%, at least 80%, at least 85%, at least 90%, at least 95% or 100% sequence identity to any of SEQ ID NO: 4.

The invention provides a cardiomyocyte derived from an iPSC, wherein the cardiomyocyte comprises a nucleic acid sequence encoding Cas12a, wherein the nucleic acid sequence has been codon optimised by the method of the invention. In some embodiments, the cardiomyocyte derived from an iPSC comprises a nucleic acid having at least 70%, at least 75%, at least 80%, at least 85%, at least 90%, at least 95% or 100% sequence identity to any of SEQ ID NO: 4.

The invention provides an iPSC comprising a nucleic acid sequence encoding Cas13Rx, wherein the nucleic acid sequence has been codon optimised by the method of the invention. In some embodiments, the iPSC comprises a nucleic acid having at least 70%, at least 75%, at least 80%, at least 85%, at least 90%, at least 95% or 100% sequence identity to any of SEQ ID NO: 5.

The invention provides a neuronal cell derived from an iPSC, wherein the neuronal cell comprises a nucleic acid sequence encoding Cas13Rx, wherein the nucleic acid sequence has been codon optimised by the method of the invention. In some embodiments, the neuronal cell derived from an iPSC comprises a nucleic acid having at least 70%, at least 75%, at least 80%, at least 85%, at least 90%, at least 95% or 100% sequence identity to any of SEQ ID NO: 5.

The invention provides a hepatocyte derived from an iPSC, wherein the hepatocyte comprises a nucleic acid sequence encoding Cas13Rx, wherein the nucleic acid sequence has been codon optimised by the method of the invention. In some embodiments, the hepatocyte derived from an iPSC comprises a nucleic acid having at least 70%, at least 75%, at least 80%, at least 85%, at least 90%, at least 95% or 100% sequence identity to any of SEQ ID NO: 5.

The invention provides a macrophage derived from an iPSC, wherein the macrophage comprises a nucleic acid sequence encoding Cas13Rx, wherein the nucleic acid sequence has been codon optimised by the method of the invention. In some embodiments, the macrophage derived from an iPSC comprises a nucleic acid having at least 70%, at least 75%, at least 80%, at least 85%, at least 90%, at least 95% or 100% sequence identity to any of SEQ ID NO: 5.

The invention provides a cardiomyocyte derived from an iPSC, wherein the cardiomyocyte comprises a nucleic acid sequence encoding Cas13Rx, wherein the nucleic acid sequence has been codon optimised by the method of the invention. In some embodiments, the cardiomyocyte derived from an iPSC comprises a nucleic acid having at least 70%, at least 75%, at least 80%, at least 85%, at least 90%, at least 95% or 100% sequence identity to any of SEQ ID NO: 5.

### EXAMPLES

The invention will be further clarified by the following non-limiting examples.

### Example 1

### Cas9 Silencing in Differentiated Cells

Multiple approaches have been used in an attempt to overcome Cas9 silencing during iPSC differentiation. Such approaches include integrating multiple copies of Cas9 into host cell genomes using lentivirus/transposons; testing Cas9 expression under various mammalian expression promoters; and targeting Cas9 to genomic safe harbour sites. Despite these efforts, researchers have observed that Cas9 protein levels dramatically decrease in differentiated cells compared to levels observed in iPSCs. Interestingly, despite a decrease in protein levels, Cas9 mRNA levels remain detectable during differentiation.

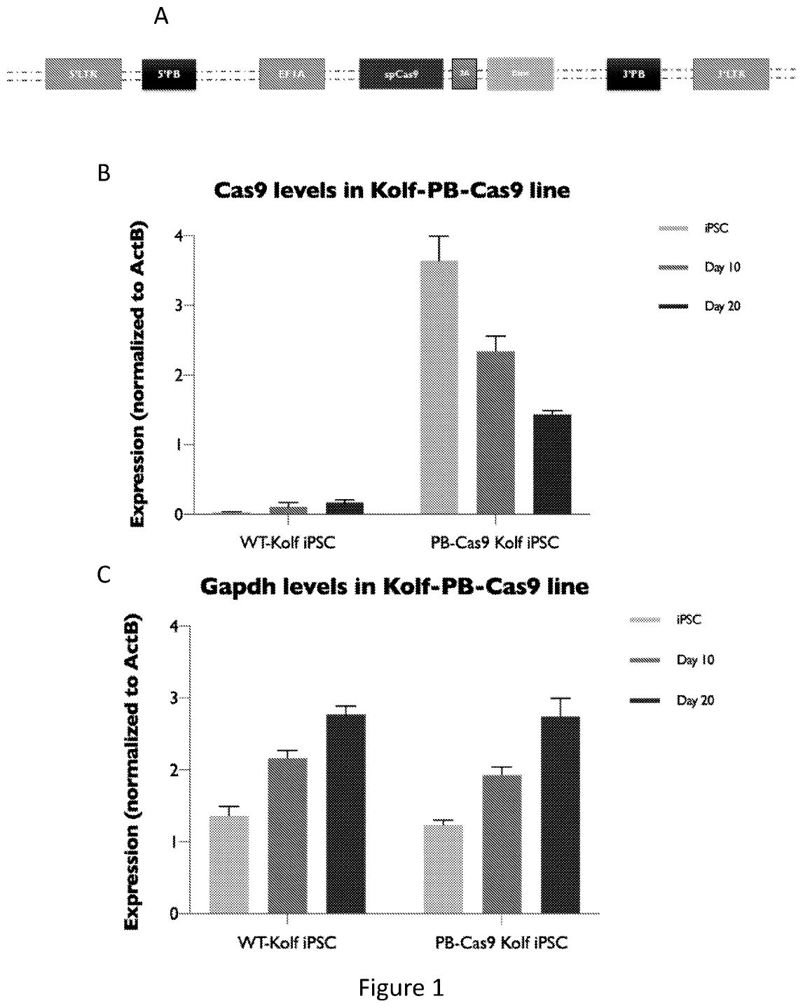

The inventors first confirmed that Cas9 expression decreases during differentiation of iPSCs (FIG. 1). A stable Cas9 expressing iPSC was generated using PiggyBac transposase (plasmid schematic FIG. 1A), and then differentiated to dopaminergic neurons. RNA collected at Day 10 and Day 20 was analysed for Cas9 expression and GAPDH expression during differentiation. Cas9 mRNA decreased by approx. 60% by Day 20 (FIG. 1B).

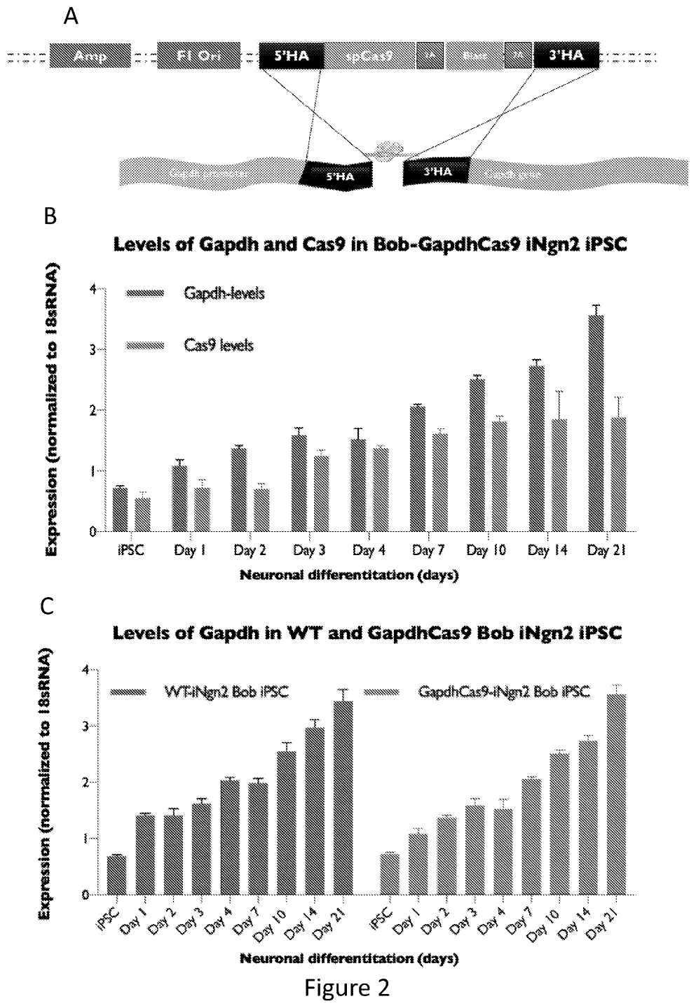

In an attempt to circumvent Cas9 silencing, the inventors generated a Bob-iNgn2 GAPDH-Cas9 iPSC line. Cas9 was inserted at the site of housekeeping gene GAPDH to ensure continued transcription. GAPDH was selected as a good housekeeping gene for knocking-in Cas9 because GAPDH levels were shown to be gradually increasing during iPSC derived neuronal differentiation protocol (FIG. 1C). Homology directed recombination was used to knock-in Cas9 just upstream of GAPDH (FIG. 2A).

Cas9 and GAPDH mRNA and protein levels were assessed during cortical neuron differentiation, which is rapid (14 days) and driven using the inducible Ngn2 transgene. Results from RT-qPCR for mRNA levels demonstrated expression of Cas9 comparable to that of GAPDH during iPSC differentiation to neurons (FIG. 2B), which was encouraging in comparison to results obtained from Cas9 integrated randomly using transposons as seen previously.

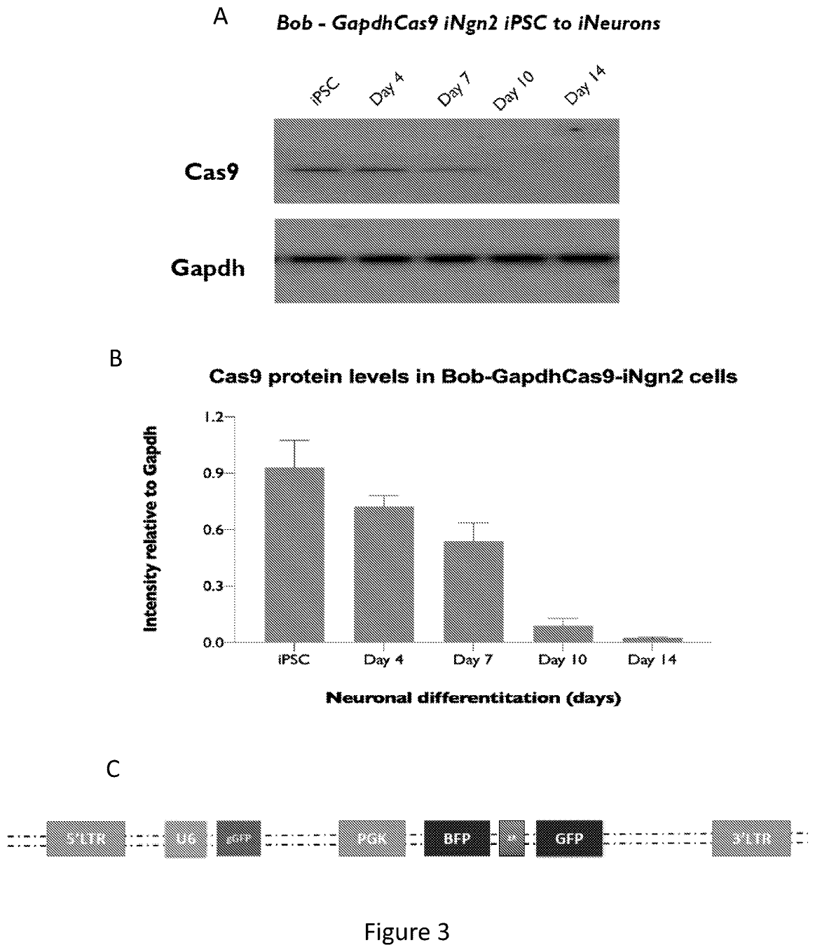

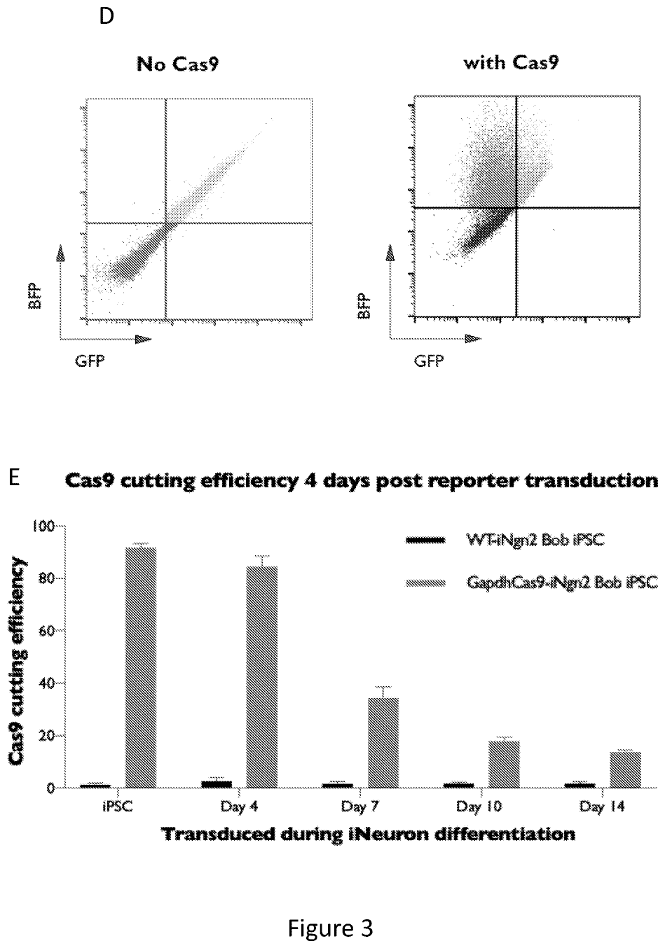

Cas9 protein levels were determined using Western blotting (FIGS. 3A and 3B) and Cas9 nuclease activity was determined using a fluorescence reporter construct (FIGS. 3C-3E). Despite encouraging evidence at mRNA level, the cells lines showed loss of Cas9 protein and therefore loss of Cas9 activity after 4 days in differentiation media. Thus, despite successful knock-in at endogenous GAPDH gene, the housekeeping gene's promoter was unable to maintain constitutive expression of Cas9 protein during differentiation of iPSCs to neuronal cell types.

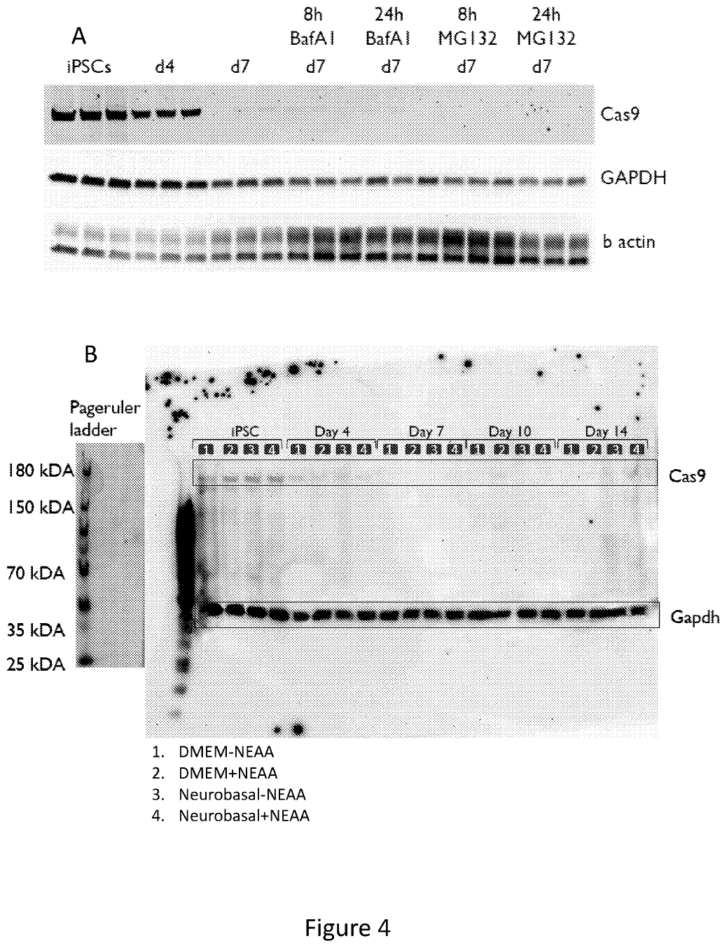

To determine whether Cas9 silencing at protein levels was the result of protein degradation through either proteasomes or the autophagy pathway in differentiated neurons, the inventors blocked proteasome degradation using MG132 inhibitor and blocked the autophagy-lysosome pathway using Bafilomycin A1 (BafA1). Experiments on Day 7 neurons, where Cas9 levels appeared to be reduced by 60% compared to Day 4, showed that blocking these protein degradation pathways fails to rescue Cas9 levels (FIG. 4A).

Given that Cas9 levels appeared to drop dramatically between Day 4 and Day 7 of cortical neuronal differentiation, which coincided with a change in differentiation media according to the cortical neuron generation protocol, the inventors additionally tested alternative protocols during differentiation to determine if Cas9 levels dropped in the presence of media or if it could be rescued by retaining supplements used from Day 0 to Day 4 of the protocol. These experiments demonstrated that Cas9 silencing could not be rescued by altering the media composition (FIG. 4B).

### Cas9 Codon Optimisation

The presence of Cas9 mRNA, but lack of Cas9 protein suggests that Cas9 transcription is uncoupled from Cas9 translation. With Cas9 silencing evident particularly during Day 4 to Day 7 of neuronal differentiation, the inventors considered the neuronal phenotype of cells to be hindering Cas9 expression. Considerable evidence demonstrates that synonymous codon choices in natural mRNAs have evolved in response to diverse selective pressures at both the RNA and protein levels. The inventors therefore hypothesized that Cas9 may require further codon optimization to be functional in differentiated cell types.

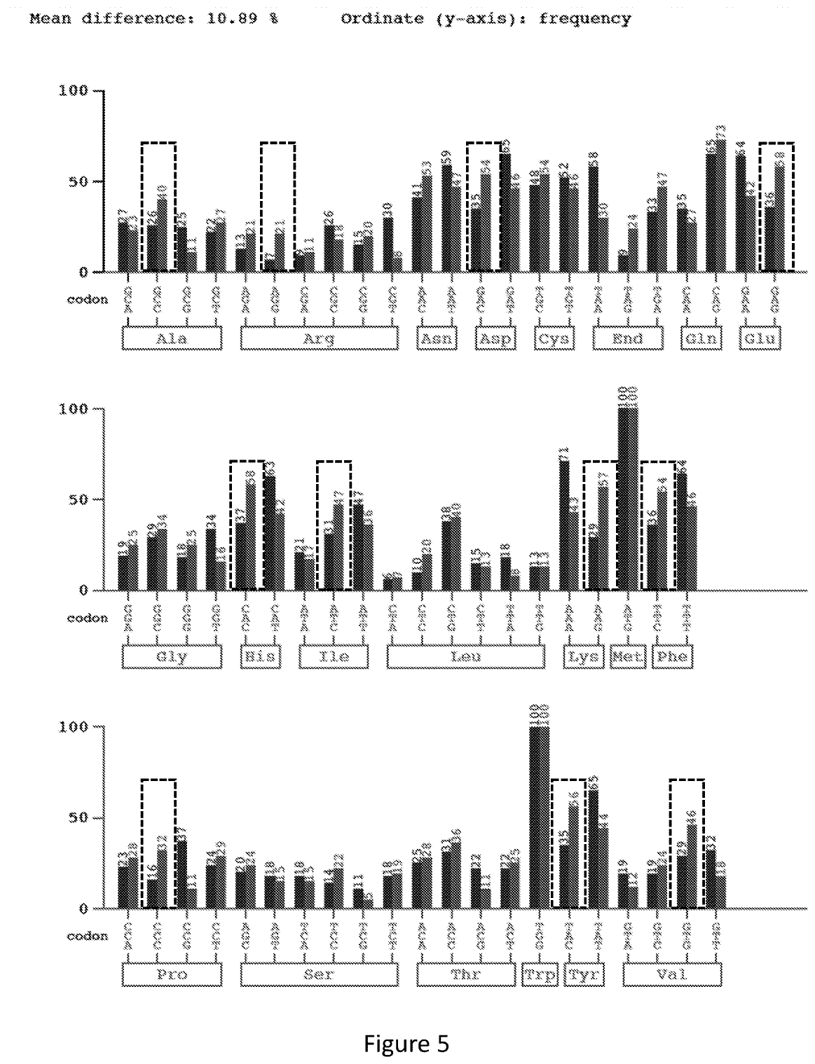

Generic codon usage frequencies of E. coli and humans (obtained from Codon Usage Database by Kazusa (Nakamura, Y. et al. Nucleic acids research 2000 28(1):292)—available at https://www.kazusa.or.jp/codon/) were compared (FIG. 5). The comparison showed that codon usage between the two organisms is fairly comparable. However amino acids such as Asp, Arg, His, Val, and others that have been highlighted using dashed boxes, show significant differences in their use in humans compared to bacteria. These small differences amount to substantial changes in protein translation and the effect is compounded in large proteins such as Cas9 which has over 1000 codons.

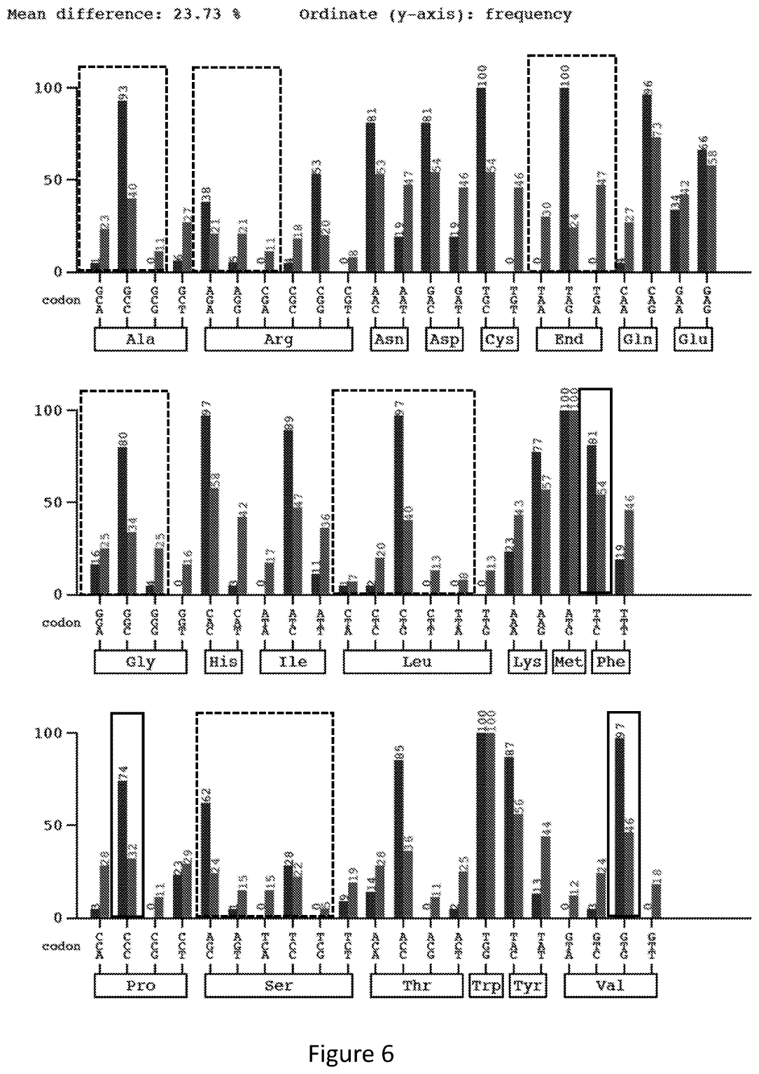

The existing Cas9 (old-Cas9) sequence (SEQ ID NO: 2) is optimized for expression in human cells based on the existing gold standard method based on human codon usage frequency (FIG. 6).

The inventors sought to determine whether differentiated neurons that are post-mitotic in nature exhibit codon biases that differ from human generic codon biases by determining the codon usage frequency of the highly expressed neuronal marker tubulin III (Tuj1). The inventors analysed the codon distribution of an established protein coding transcript of tubulin III (Ensembl Transcript: TUBB3-208 ENST00000555576.5; SEQ ID NO: 8) using the codon calculator tool available at https://www.biologicscorp.com/tools/CodonUsageCalculator/. The codon usage frequency of tubulin III was compared to human generic codon usage (FIG. 7).

The codon usage frequency of tubulin III showed that tubulin III's codon preference is different to human generic codon usage. Key differences are highlighted by dashed boxes (no usage) and triangles (high usage) (FIG. 7).

Using the codon usage frequency of tubulin III, a novel codon optimised Cas9 variant with altered codons was generated (CodOpt-Cas9). The DNA sequence of the codon optimised Cas9 is provided below with codons that have been altered highlighted in bold:

(SEQ ID NO: 1)
ATG GAC AAG AAG TAC TCT ATC GGC CTG GAC ATC GGC ACC AAC AGC GTG GGC
TGG GCC GTC ATC ACC GAC GAG TAC AAG GTG CCT TCT AAG AAG TTC AAG GTG
CTG GGC AAC ACC GAC CGC CAT TCT ATC AAG AAG AAC CTG ATC GGC GCC CTG
CTG TTC GAC TCT GGC GAG ACC GCC GAG GCC ACC AGA CTG AAG CGG ACC GCC
CGA CGC CGA TAC ACC AGA CGG AAG AAC AGA ATC TGC TAC CTT CAG GAG ATC
TTC AGC AAC GAG ATG GCC AAG GTG GAC GAC TCT TTC TTC CAT CGC CTG GAG
GAG AGC TTC CTG GTG GAG GAG GAC AAG AAG CAT GAG CGC CAT CCT ATC TTC
GGC AAC ATC GTG GAC GAG GTG GCC TAC CAT GAG AAG TAC CCT ACC ATC TAC
CAT CTG AGG AAG AAG CTG GTG GAC TCT ACG GAC AAG GCC GAC CTG AGA CTT
ATC TAC CTG GCC CTG GCC CAT ATG ATC AAG TTC CGG GGC CAT TTC CTC ATC
GAG GGC GAC CTC AAC CCC GAC AAC AGC GAC GTG GAC AAG CTG TTC ATC CAG
TTG GTG CAG ACC TAC AAC CAG CTT TTC GAG GAG AAC CCC ATC AAC GCC TCT
GGC GTG GAC GCC AAG GCC ATC CTG AGT GCC CGC CTG TCT AAG AGC CGC AGA
CTT GAG AAC CTG ATC GCC CAG CTG CCC GGC GAG AAG AAG AAC GGC CTG TTC
GGC AAC CTT ATC GCC CTG TCT CTG GGC CTT ACC CCT AAC TTC AAG TCT AAC
TTC GAC CTG GCC GAG GAC GCC AAG CTG CAG CTT AGC AAG GAC ACC TAC GAC
GAC GAC TTG GAC AAC CTG CTT GCC CAG ATC GGC GAC CAG TAC GCC GAC CTG
TTC CTG GCC GCC AAG AAC TTG AGC GAC GCC ATC CTG CTT AGC GAC ATC CTG
AGA GTC AAC ACC GAG ATC ACC AAG GCC CCT CTG TCT GCC AGC ATG ATC AAG
CGG TAC GAC GAG CAT CAC CAG GAC CTG ACC CTG TTG AAG GCC CTC GTG CGA
CAG CAG CTG CCT GAG AAG TAC AAG GAG ATC TTC TTT GAC CAG AGC AAG AAC
GGC TAC GCC GGC TAC ATC GAC GGC GGC GCC TCT CAG GAG GAG TTC TAC AAG
TTC ATC AAG CCC ATC CTG GAG AAG ATG GAC GGC ACC GAG GAG CTT CTG GTC
AAG CTG AAC AGG GAG GAC CTG CTT AGG AAG CAG CGC ACC TTC GAC AAC GGC
TCA ATC CCT CAT CAG ATC CAC CTG GGC GAG TTG CAT GCC ATC CTC AGA CGC
CAG GAG GAC TTC TAC CCC TTC CTG AAG GAC AAC AGG GAG AAG ATC GAG AAG
ATC CTG ACC TTC CGA ATC CCC TAC TAC GTG GGC CCT CTG GCC CGA GGC AAC
TCT CGA TTC GCC TGG ATG ACC CGC AAG TCT GAG GAG ACC ATC ACC CCT TGG
AAC TTC GAG GAG GTC GTG GAC AAG GGC GCC TCT GCC CAG TCA TTC ATC GAG
CGG ATG ACC AAC TTC GAC AAG AAC CTG CCC AAC GAG AAG GTG CTG CCT AAG
CAT TCT TTG CTG TAC GAG TAC TTC ACC GTG TAC AAC GAG CTG ACC AAG GTG
AAG TAC GTG ACC GAG GGC ATG CGC AAG CCT GCC TTC CTG TCT GGC GAG CAG
AAG AAG GCC ATC GTG GAC CTG TTG TTC AAG ACC AAC CGG AAG GTG ACC GTG
AAG CAG CTG AAG GAG GAC TAC TTC AAG AAG ATC GAG TGC TTC GAC TCT GTG
GAG ATC AGC GGC GTG GAG GAC CGC TTC AAC GCC TCT CTG GGC ACC TAC CAT
GAC CTG TTG AAG ATC ATC AAG GAC AAG GAC TTC CTG GAC AAC GAG GAG AAC
GAG GAC ATC CTG GAG GAC ATC GTG CTG ACC TTG ACC CTG TTC GAG GAC CGG
GAG ATG ATC GAG GAG CGG CTG AAG ACC TAC GCC CAT CTG TTC GAC GAC AAG
GTG ATG AAG CAG CTG AAG CGG AGA AGG TAC ACC GGC TGG GGC AGA CTG TCT
AGA AAG CTG ATC AAC GGC ATC CGC GAC AAG CAG TCT GGC AAG ACC ATC CTG
GAC TTC CTG AAG TCT GAC GGC TTC GCC AAC CGG AAC TTC ATG CAG CTG ATC
CAT GAC GAC TCT CTG ACC TTC AAG GAG GAC ATC CAG AAG GCC CAG GTG TCT
GGC CAG GGC GAC TCT CTG CAT GAG CAT ATC GCC AAC CTG GCC GGC TCT CCC
GCC ATC AAG AAG GGC ATC CTG CAG ACC GTG AAG GTG GTC GAC GAG CTG GTG
AAG GTC ATG GGC AGG CAT AAG CCC GAG AAC ATC GTG ATC GAG ATG GCC CGC
GAG AAC CAG ACC ACC CAG AAG GGC CAG AAG AAC TCT CGG GAG AGA ATG AAG
AGG ATC GAG GAG GGC ATC AAG GAG CTG GGC TCT CAG ATC CTG AAG GAG CAT
CCT GTG GAG AAC ACC CAG CTG CAG AAC GAG AAG CTG TAC CTG TAC TAC CTG
CAG AAC GGG CGG GAC ATG TAC GTG GAC CAG GAG CTG GAC ATC AAC AGA CTC
TCT GAC TAC GAC GTT GAC CAT ATC GTG CCT CAG AGC TTC CTG AAG GAC GAC
TCT ATC GAC AAC AAG GTG CTG ACC CGC TCT GAC AAG AAC CGG GGC AAG TCT
GAC AAC GTG CCT TCT GAG GAG GTG GTG AAG AAG ATG AAG AAC TAC TGG CGC
CAG CTG CTT AAC GCC AAG CTG ATC ACC CAG AGA AAG TTC GAC AAC CTG ACC
AAG GCC GAG CGA GGC GGC CTC TCT GAG CTG GAC AAG GCC GGC TTC ATC AAG
AGA CAG CTG GTG GAG ACC AGA CAG ATC ACC AAG CAT GTG GCC CAG ATC CTG
GAC TCT AGA ATG AAC ACC AAG TAC GAC GAG AAC GAC AAG CTG ATC CGG GAG
GTG AAG GTG ATC ACC CTG AAG TCT AAG CTG GTC AGC GAC TTC CGC AAG GAC
TTC CAG TTC TAC AAG GTG AGA GAG ATC AAC AAC TAC CAT CAC GCC CAT GAC
GCC TAC CTG AAC GCC GTG GTC GGC ACC GCC TTG ATC AAG AAG TAC CCT AAG
CTG GAG TCT GAG TTC GTG TAC GGC GAC TAC AAG GTG TAC GAC GTG AGA AAG
ATG ATC GCC AAG TCT GAG CAG GAG ATC GGC AAG GCC ACC GCC AAG TAC TTC
TTC TAC TCT AAC ATC ATG AAC TTC TTC AAG ACC GAG ATC ACC CTG GCC AAC
GGC GAG ATC AGA AAG CGG CCC CTG ATC GAG ACC AAC GGC GAG ACC GGC GAG
ATC GTG TGG GAC AAG GGC AGA GAC TTC GCC ACC GTC AGA AAG GTC CTG TCT
ATG CCC CAG GTG AAC ATC GTG AAG AAG ACC GAG GTG CAG ACC GGC GGC TTC
TCT AAG GAG TCT ATC CTG CCC AAG CGG AAC AGC GAC AAG CTG ATC GCC AGA
AAG AAG GAC TGG GAC CCC AAG AAG TAC GGC GGC TTC GAC TCT CCC ACC GTG
GCC TAC TCT GTC CTG GTG GTC GCC AAG GTC GAG AAG GGC AAG TCT AAG AAG
CTG AAG TCT GTG AAG GAG CTG CTC GGC ATC ACC ATC ATG GAG AGA AGC TCT
TTC GAG AAG AAC CCT ATC GAC TTC CTG GAG GCC AAG GGC TAC AAG GAG GTG
AAG AAG GAC CTG ATC ATC AAG CTG CCC AAG TAC TCT CTG TTC GAG CTG GAG
AAC GGC CGG AAG AGA ATG CTG GCC TCT GCC GGC GAG CTG CAG AAG GGC AAC
GAG CTG GCC TTG CCT TCT AAG TAC GTG AAC TTC TTG TAC CTG GCC TCT CAC
TAC GAG AAG CTG AAG GGC TCT CCC GAG GAC AAC GAG CAG AAG CAG CTG TTC
GTG GAG CAG CAT AAG CAT TAC CTG GAC GAG ATC ATC GAG CAG ATC AGC GAG
TTC TCT AAG CGG GTG ATC CTG GCC GAC GCC AAC CTG GAC AAG GTC CTG TCT
GCC TAC AAC AAG CAT AGA GAC AAG CCC ATC AGA GAG CAG GCC GAG AAC ATC
ATC CAC CTG TTC ACC CTG ACC AAC CTG GGC GCC CCC GCC GCC TTC AAG TAC
TTC GAC ACC ACC ATC GAC AGA AAG CGG TAC ACC AGC ACC AAG GAG GTG CTC
GAC GCC ACC CTG ATC CAT CAG TCT ATC ACC GGC CTG TAC GAG ACC AGA ATC
GAC CTG AGC CAG CTG GGC GGC GAC TGA

To ensure that only the codons but not the amino acid (protein) sequence of Cas9 has been altered, the inventors verified the protein sequences resulting from both variants of Cas9 using ClustalW protein alignment tool.

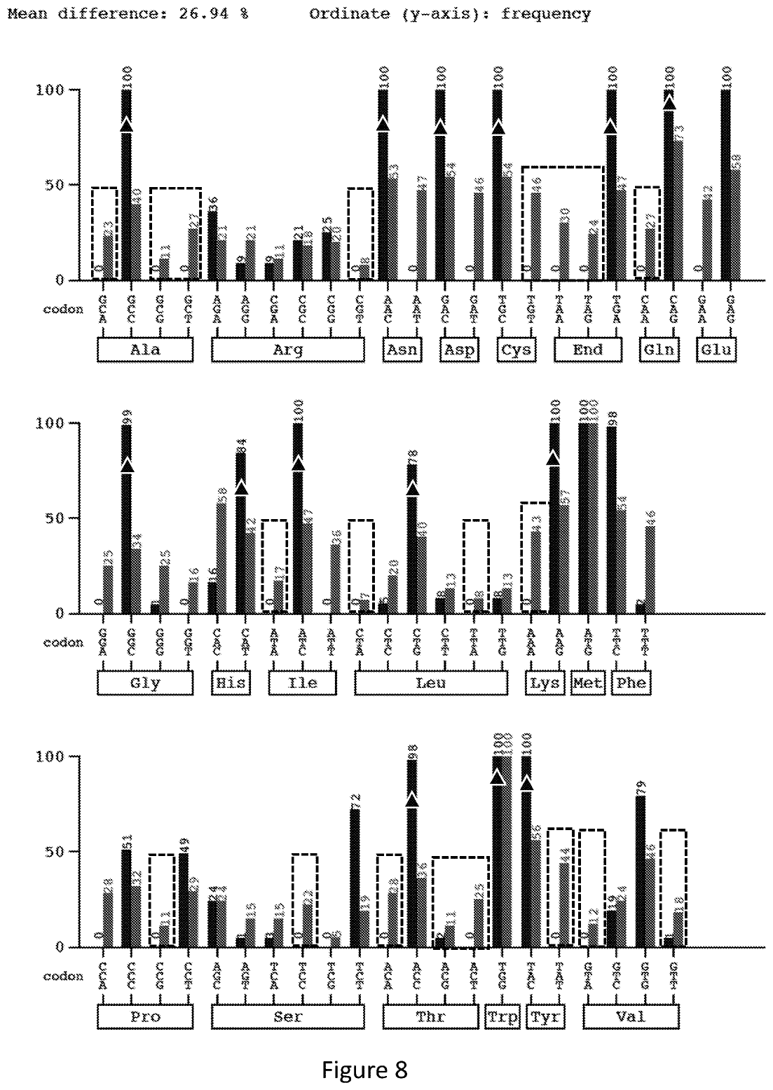

The codon usage frequency of CodOpt-Cas9 was compared to human generic codon usage (FIG. 8). This comparison demonstrated that codon optimising Cas9 using the codon biases of tubulin III resulted in a sequence having substantially different codon usage frequencies compared to human generic codon usage.

### CodOpt-Cas9 Expression and Activity

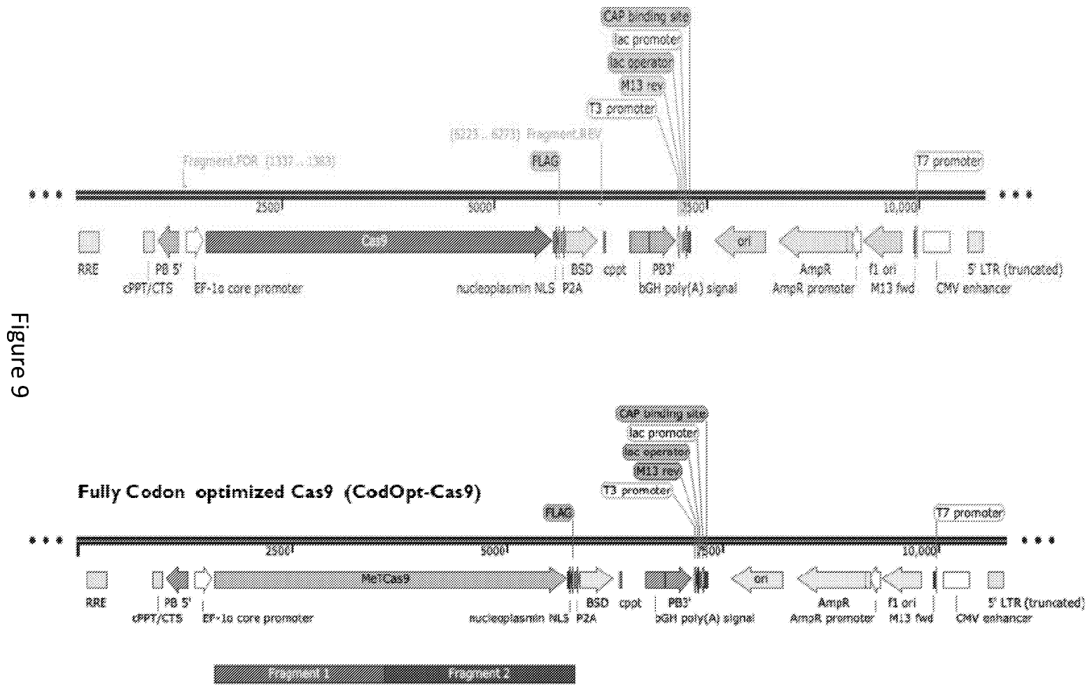

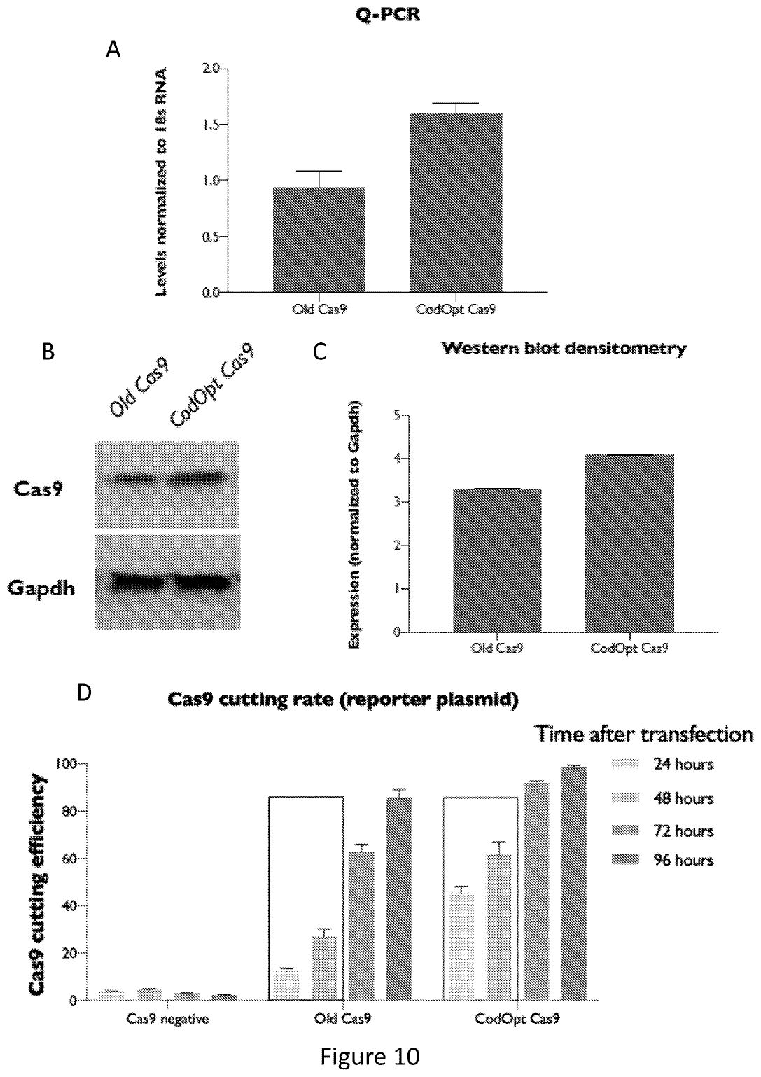

The inventors cloned the codon optimized Cas9 into an expression construct that is directly comparable to the old-Cas9 expression construct (FIG. 9). Initially, the inventors tested and compared old-Cas9 and CodOpt-Cas9 expression in HEK293 cells. Advantageously, these experiments demonstrated that CodOpt-Cas9 had increased expression at both mRNA and protein levels compared to the old-Cas9 (FIG. 10).

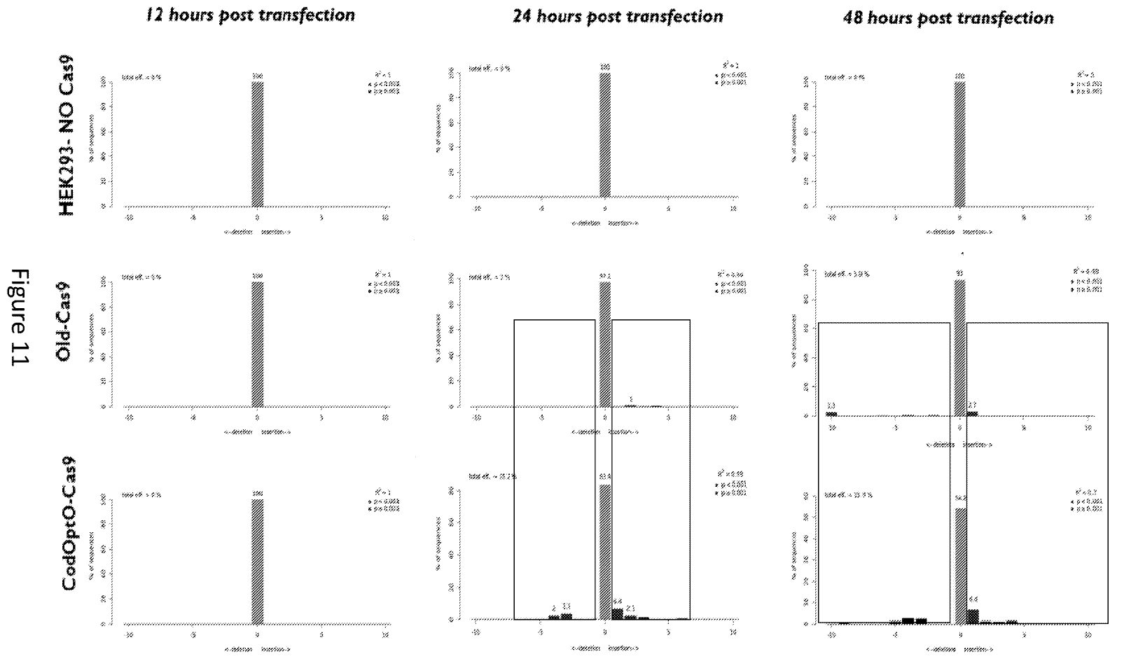

HEK293 cells harbouring each of these two variants of Cas9 were used to perform a Cas9 activity assay using a fluorescence reporter plasmid. The results for these Cas9 cutting assays demonstrate that CodOpt-Cas9 displays a higher nuclease activity and starts editing much faster than old-Cas9 (FIG. 10D). This faster cutting efficiency could also be observed through formation of indels when Cas9 was guided to edit a non-essential gene (ST6GALNAC6) in the genome (FIG. 11). These results indicate that CodOpt-Cas9 achieves higher expression than old-Cas9 and exhibits faster and more efficient cutting.

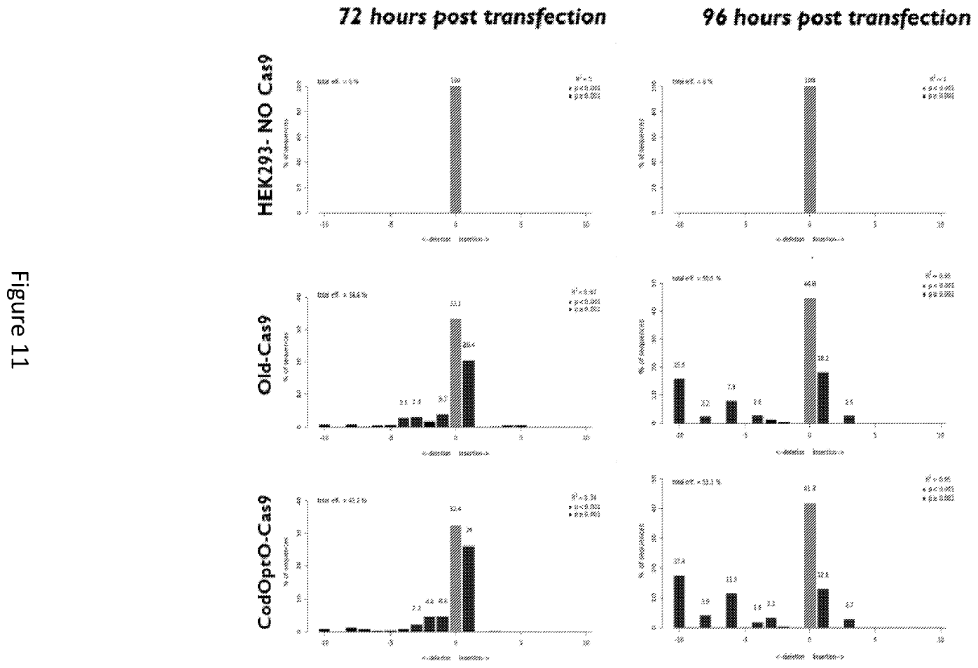

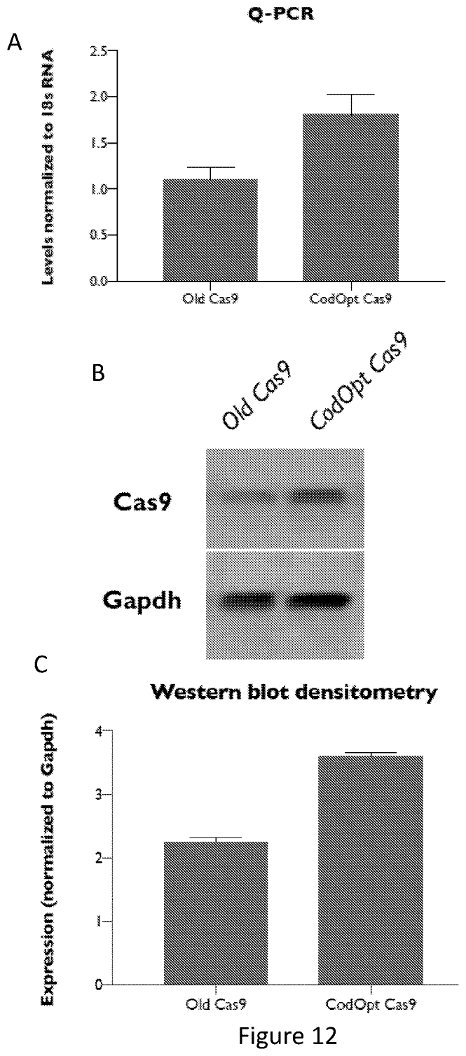

Next, the inventors attempted to express CodOpt-Cas9 in Bob-iNgn2 iPSCs. A CodOpt-Cas9 line was generated using PiggyBac transposase. Cas9 expression was checked at both mRNA and protein levels. Similar to HEK293 cells, iPSCs harbouring CodOpt-Cas9 exhibited high levels of Cas9 mRNA and protein (FIG. 12).

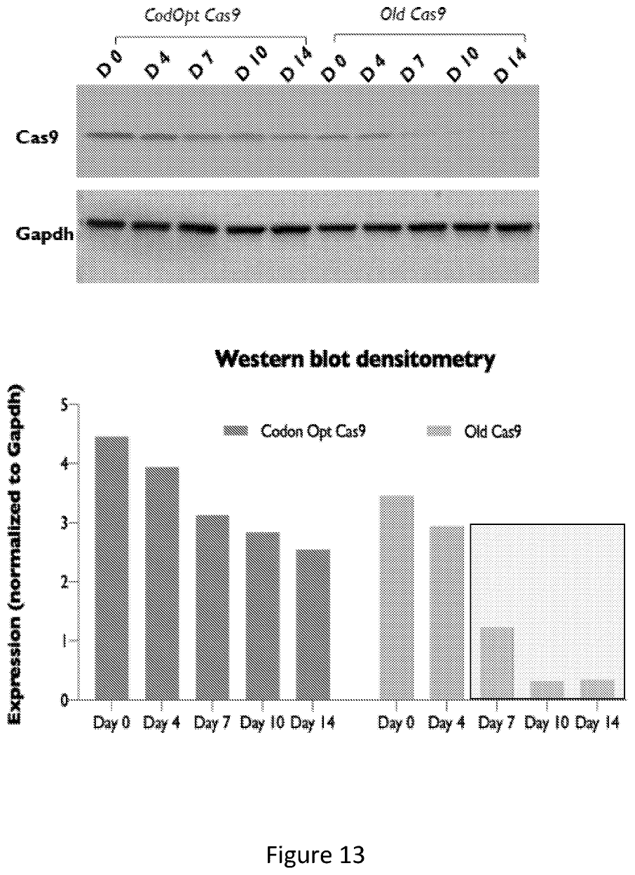

Bob-iNgn2 iPSCs containing either CodOpt-Cas9 or old-Cas9 were then differentiated to cortical neurons. Western blotting for protein levels of Cas9 showed that Cas9 could be easily detected in differentiated neuronal cells expressing CodOpt-Cas9 (FIG. 13). However, as observed in previous experiments, cells expressing old-Cas9 showed a sharp decrease in Cas9 levels as cells entered a more neuronal phenotype (FIG. 13).

These results indicate that optimising the codon usage of Cas9 to mirror the codon usage of a highly expressed neuronal marker protein, tubulin III, significantly improves the expression of Cas9 in iPSCs and in iPSCs derived neurons. Advantageously, Cas9 expression was sustained throughout differentiation to neurons which significantly improves the potential research applications of both iPSC derived cell lines and the CRISPR-Cas9 system (FIG. 13).

### Codon Optimisation as a Tool to Control Levels of Expression

Codon usage has recently been spotlighted as a key determinant of translation elongation rates and co-translational protein folding, with preferred codons enhancing translational efficiency and folding fidelity. The unequal usage of synonymous codons, referred as codon bias and the universal nature of this bias, from yeast to humans, suggests the existence of a secondary code within the more familiar genetic code. This secondary code is emerging as a major regulator of translational speed and co-translational protein folding and thereby a significant determinant of the cellular levels of specific proteins.

Based on the observation that CodOpt-Cas9 achieved better expression than old-Cas9 in HEK293 cells and iPSCs at both the mRNA and protein level, the inventors tested whether levels of Cas9 could be tuned through partial codon optimization. A Cas9 variant was produced wherein the first 606 amino acid codons were optimized based on tubulin III codon usage, while the remaining codons were unaltered. This version of Cas9, which encodes a protein wherein the N-terminal region is codon optimised, is represented by SEQ ID NO: 3, and is referred to herein as NOpt-Cas9.

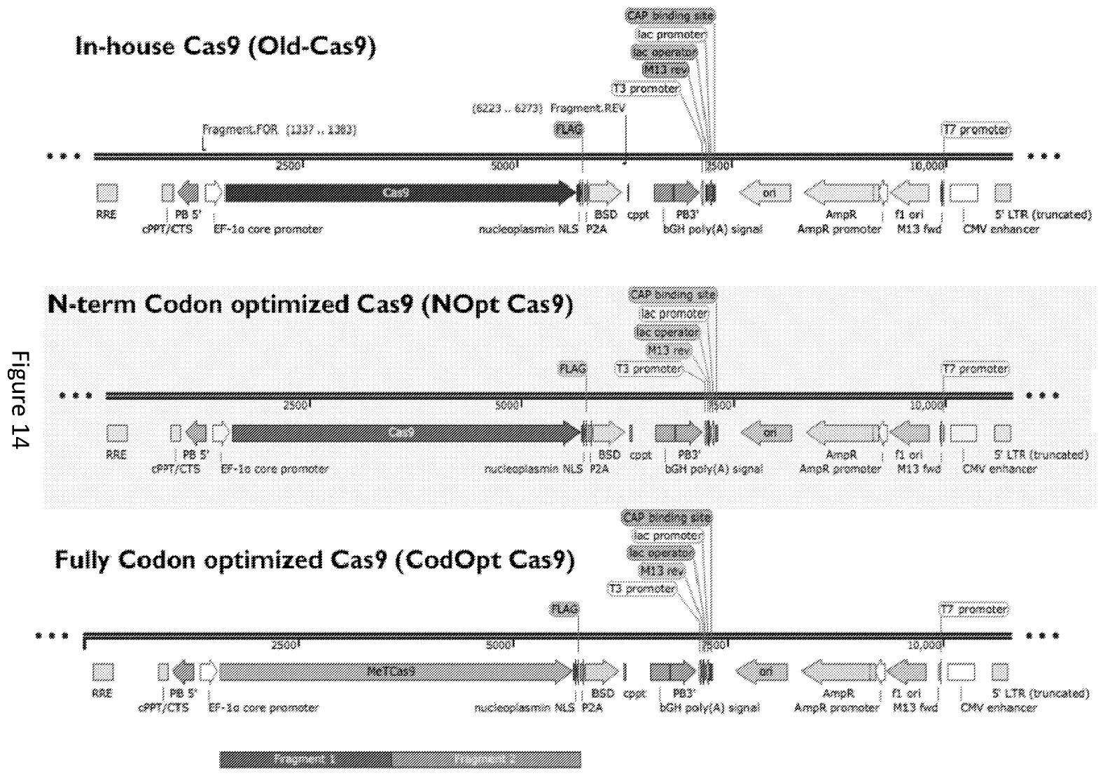

Bob iPSC cell lines comprising old-Cas9, CodOpt-Cas9 and NOpt-Cas9 were generated using PiggyBac integration (FIG. 14) and then differentiated to neurons. Cas9 cutting efficiency was determined in the iPSC stage and through various stages of neuron differentiation.

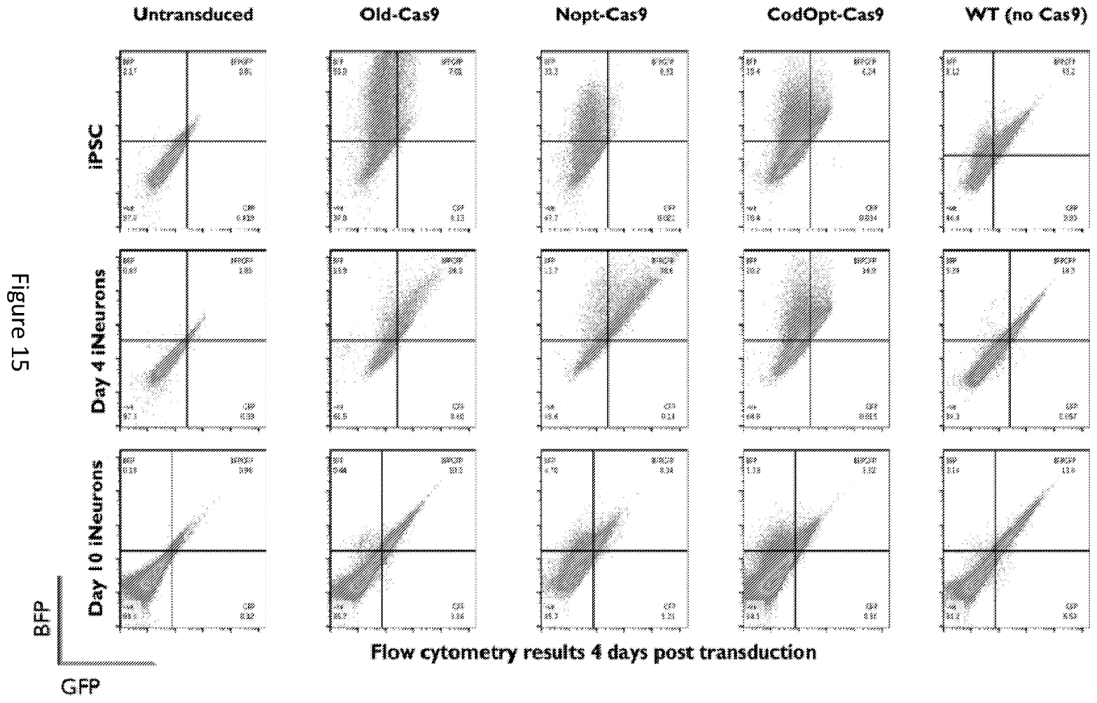

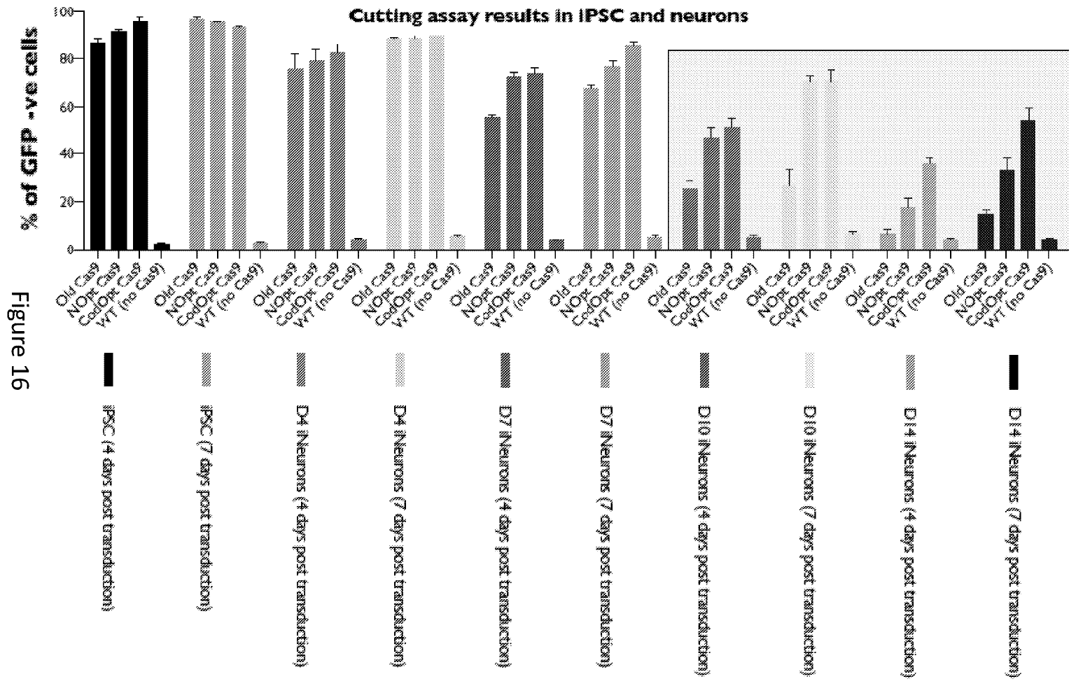

NOpt-Cas9 and CodOpt-Cas9 were found to have better cutting efficiency than the Old Cas9 as cells progress to neuronal fate (FIG. 15). Interestingly, experiments performed in more differentiated cells (day 10 and day 14 neurons) demonstrated that the cutting efficiency of NOpt-Cas9 dropped slightly in comparison to CodOpt-Cas9. Despite this drop in the editing efficiency, NOpt-Cas9 did exhibit higher cutting than old-Cas9 at these time points (FIGS. 15 and 16). Cutting efficiencies were assessed 4 days and 7 days after cell transductions.

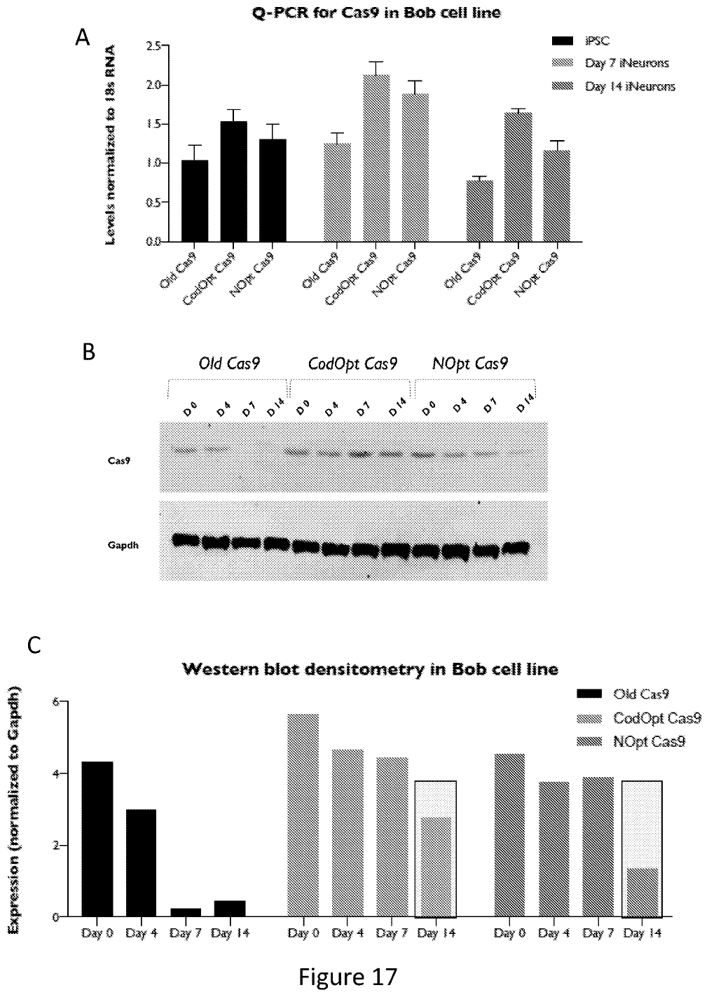

The inventors also assessed Cas9 expression at mRNA and protein levels to determine how partial optimization affects transcription and translation. Both complete and partial codon optimization of Cas9 results in increased mRNA levels and sustained expression during differentiation (FIG. 17A). It was interesting to note that in cell lines containing NOpt-Cas9, the levels of Cas9 decreased significantly post day 7 in neurons (FIGS. 17B and C; boxes highlight comparison). While this is reflected by reduced editing efficiency by NOpt-Cas9, it suggests that the altered codons contribute to sustained protein expression as the neurons mature in vitro.

Cas9 Expression in Non-Neuronal iPSC Derived Cell Types

Similar to iPSC derived neurons, robust and sustained expression of Cas9 has not previously been achieved in other iPSC derived cells types such as hepatocytes and macrophages. The inability to perform a CRISPR-Cas9 genome wide screen therefore limits the use of these cell lines to their progenitor state similar to the limitations observed in performing a Cas9 screen in differentiated neurons. The inventors therefore set out to determine if Cas9 expression could be achieved when the CodOpt-Cas9 iPSC line was differentiated to other cell types.

Hepatocytes were derived from iPSCs based on the protocol established by (Hannan et al. Nature protocols. 8, 430-437 (2013)). Similar to differentiating neurons, Cas9 levels have been observed to drop sharply after Day 7 of differentiation as the cells undergo multiple morphological changes before committing to epithelial lineage.

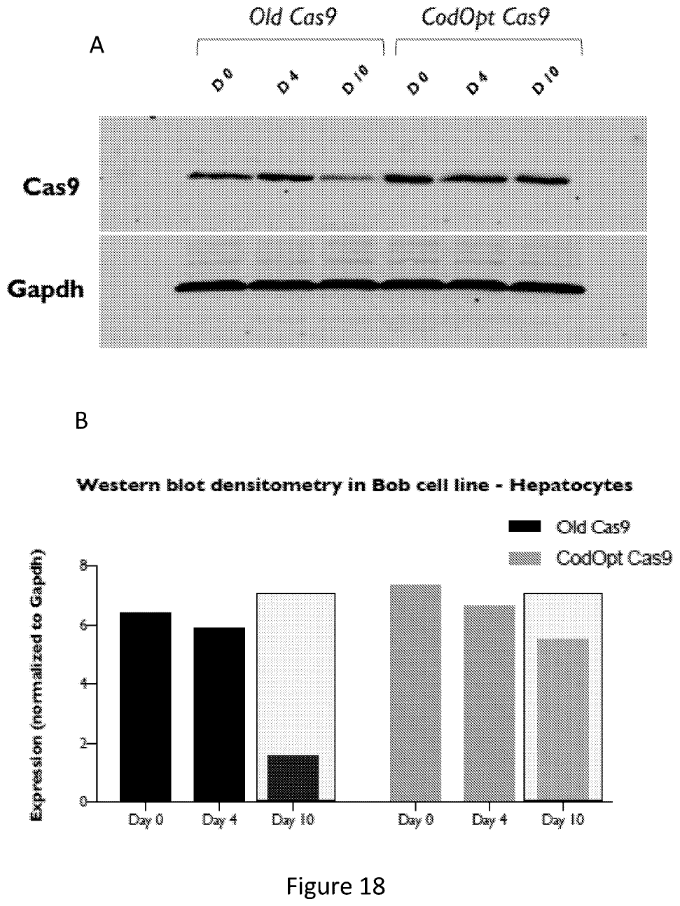

Bob iPSC cells harbouring either old-Cas9 or CodOpt-Cas9 were differentiated into hepatocytes and cell pellets were collected on Days 0, 4 and 10 of differentiation. Western blotting revealed that CodOpt-Cas9 levels were significantly higher than levels of old-Cas9 in iPSC derived hepatocyte like cells, specifically post Day 7 (FIG. 18).

These results demonstrate that CodOpt-Cas9 is able to achieve and maintain high expression levels in iPSC derived cells other than neurons. Advantageously, these results demonstrate that significant improvements in expression of a target nucleic acid may be enjoyed across a variety of cell types, including cells that do not normally express the gene encoding the highly expressed protein on which codon optimization is based.

### SUMMARY

These results indicate that codon optimizing a target nucleic acid using the codon biases of a gene encoding a highly expression protein significantly improves the expression of that nucleic acid in a range of cell types, even those that do not express the highly expressed gene. In addition, target nucleic acids can be partially codon optimized to regulate the level of expression. Thus, the methods described herein can be used as a solution to overcome Cas9 silencing and allow CRISPR-Cas9 genome-wide screens to be performed in various cell lines, including differentiated cell types.

### Example 2

### Codon Optimisation of Cas12a and Cas13Rx

In addition to Cas9, Cas12a and Cas13Rx have emerged as promising tools for gene editing. These CRISPR Cas proteins have been used for editing DNA and RNA respectively, thereby increasing the potential of gene-editing technology considerably. The inventors analysed the existing variants of Cas12a and Cas13Rx to determine if codon optimization had been adequately performed for human mammalian cells.

### Codon Optimised Cas12a

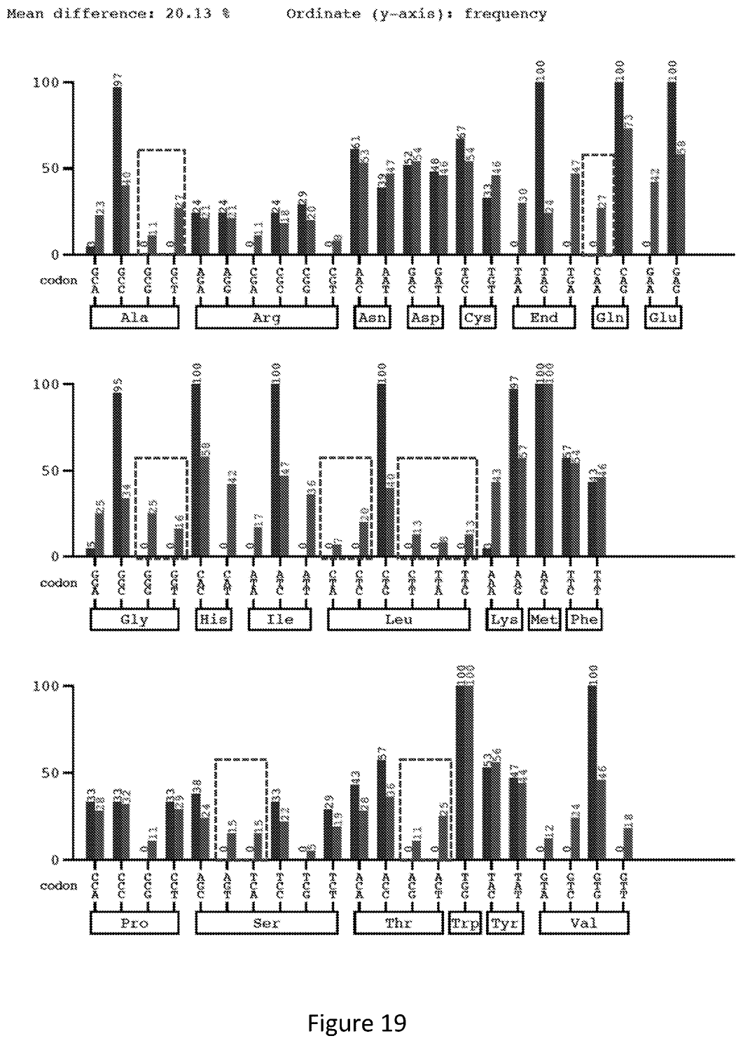

Codon usage for the existing variant of the Cas12a was based on the existing gold standard with optimisation patterns similar to those observed in old-Cas9 (FIG. 19). The starting Cas12a sequence was obtained from addgene plasmid IDs: 160573 and 78744 and is represented by SEQ ID NO: 6:

(SEQ ID NO: 6)
ATGAGCAAGCTGGAGAAGTTTACAAACTGCTACTCCCTGTCTAAGACCCT
GAGGTTCAAGGCCATCCCTGTGGGCAAGACCCAGGAGAACATCGACAATA
AGCGGCTGCTGGTGGAGGACGAGAAGAGAGCCGAGGATTATAAGGGCGTG
AAGAAGCTGCTGGATCGCTACTATCTGTCTTTTATCAACGACGTGCTGCA
CAGCATCAAGCTGAAGAATCTGAACAATTACATCAGCCTGTTCCGGAAGA
AAACCAGAACCGAGAAGGAGAATAAGGAGCTGGAGAACCTGGAGATCAAT
CTGCGGAAGGAGATCGCCAAGGCCTTCAAGGGCAACGAGGGCTACAAGTC
CCTGTTTAAGAAGGATATCATCGAGACAATCCTGCCAGAGTTCCTGGACG
ATAAGGACGAGATCGCCCTGGTGAACAGCTTCAATGGCTTTACCACAGCC
TTCACCGGCTTCTTTGATAACAGAGAGAATATGTTTTCCGAGGAGGCCAA
GAGCACATCCATCGCCTTCAGGTGTATCAACGAGAATCTGACCCGCTACA
TCTCTAATATGGACATCTTCGAGAAGGTGGACGCCATCTTTGATAAGCAC
GAGGTGCAGGAGATCAAGGAGAAGATCCTGAACAGCGACTATGATGTGGA
GGATTTCTTTGAGGGCGAGTTCTTTAACTTTGTGCTGACACAGGAGGGCA
TCGACGTGTATAACGCCATCATCGGCGGCTTCGTGACCGAGAGCGGCGAG
AAGATCAAGGGCCTGAACGAGTACATCAACCTGTATAATCAGAAAACCAA
GCAGAAGCTGCCTAAGTTTAAGCCACTGTATAAGCAGGTGCTGAGCGATC
GGGAGTCTCTGAGCTTCTACGGCGAGGGCTATACATCCGATGAGGAGGTG
CTGGAGGTGTTTAGAAACACCCTGAACAAGAACAGCGAGATCTTCAGCTC
CATCAAGAAGCTGGAGAAGCTGTTCAAGAATTTTGACGAGTACTCTAGCG
CCGGCATCTTTGTGAAGAACGGCCCCGCCATCAGCACAATCTCCAAGGAT
ATCTTCGGCGAGTGGAACGTGATCCGGGACAAGTGGAATGCCGAGTATGA
CGATATCCACCTGAAGAAGAAGGCCGTGGTGACCGAGAAGTACGAGGACG
ATCGGAGAAAGTCCTTCAAGAAGATCGGCTCCTTTTCTCTGGAGCAGCTG
CAGGAGTACGCCGACGCCGATCTGTCTGTGGTGGAGAAGCTGAAGGAGAT
CATCATCCAGAAGGTGGATGAGATCTACAAGGTGTATGGCTCCTCTGAGA
AGCTGTTCGACGCCGATTTTGTGCTGGAGAAGAGCCTGAAGAAGAACGAC
GCCGTGGTGGCCATCATGAAGGACCTGCTGGATTCTGTGAAGAGCTTCGA
GAATTACATCAAGGCCTTCTTTGGCGAGGGCAAGGAGACAAACAGGGACG
AGTCCTTCTATGGCGATTTTGTGCTGGCCTACGACATCCTGCTGAAGGTG
GACCACATCTACGATGCCATCCGCAATTATGTGACCCAGAAGCCCTACTC
TAAGGATAAGTTCAAGCTGTATTTTCAGAACCCTCAGTTCATGGGCGGCT
GGGACAAGGATAAGGAGACAGACTATCGGGCCACCATCCTGAGATACGGC
TCCAAGTACTATCTGGCCATCATGGATAAGAAGTACGCCAAGTGCCTGCA
GAAGATCGACAAGGACGATGTGAACGGCAATTACGAGAAGATCAACTATA
AGCTGCTGCCCGGCCCTAATAAGATGCTGCCAAAGGTGTTCTTTTCTAAG
AAGTGGATGGCCTACTATAACCCCAGCGAGGACATCCAGAAGATCTACAA
GAATGGCACATTCAAGAAGGGCGATATGTTTAACCTGAATGACTGTCACA
AGCTGATCGACTTCTTTAAGGATAGCATCTCCCGGTATCCAAAGTGGTCC
AATGCCTACGATTTCAACTTTTCTGAGACAGAGAAGTATAAGGACATCGC
CGGCTTTTACAGAGAGGTGGAGGAGCAGGGCTATAAGGTGAGCTTCGAGT
CTGCCAGCAAGAAGGAGGTGGATAAGCTGGTGGAGGAGGGCAAGCTGTAT
ATGTTCCAGATCTATAACAAGGACTTTTCCGATAAGTCTCACGGCACACC
CAATCTGCACACCATGTACTTCAAGCTGCTGTTTGACGAGAACAATCACG
GACAGATCAGGCTGAGCGGAGGAGCAGAGCTGTTCATGAGGCGCGCCTCC
CTGAAGAAGGAGGAGCTGGTGGTGCACCCAGCCAACTCCCCTATCGCCAA
CAAGAATCCAGATAATCCCAAGAAAACCACAACCCTGTCCTACGACGTGT
ATAAGGATAAGAGGTTTTCTGAGGACCAGTACGAGCTGCACATCCCAATC
GCCATCAATAAGTGCCCCAAGAACATCTTCAAGATCAATACAGAGGTGCG
CGTGCTGCTGAAGCACGACGATAACCCCTATGTGATCGGCATCGATAGGG
GCGAGCGCAATCTGCTGTATATCGTGGTGGTGGACGGCAAGGGCAACATC
GTGGAGCAGTATTCCCTGAACGAGATCATCAACAACTTCAACGGCATCAG
GATCAAGACAGATTACCACTCTCTGCTGGACAAGAAGGAGAAGGAGAGGT
TCGAGGCCCGCCAGAACTGGACCTCCATCGAGAATATCAAGGAGCTGAAG
GCCGGCTATATCTCTCAGGTGGTGCACAAGATCTGCGAGCTGGTGGAGAA
GTACGATGCCGTGATCGCCCTGGAGGACCTGAACTCTGGCTTTAAGAATA
GCCGCGTGAAGGTGGAGAAGCAGGTGTATCAGAAGTTCGAGAAGATGCTG
ATCGATAAGCTGAACTACATGGTGGACAAGAAGTCTAATCCTTGTGCAAC
AGGCGGCGCCCTGAAGGGCTATCAGATCACCAATAAGTTCGAGAGCTTTA
AGTCCATGTCTACCCAGAACGGCTTCATCTTTTACATCCCTGCCTGGCTG
ACATCCAAGATCGATCCATCTACCGGCTTTGTGAACCTGCTGAAAACCAA
GTATACCAGCATCGCCGATTCCAAGAAGTTCATCAGCTCCTTTGACAGGA
TCATGTACGTGCCCGAGGAGGATCTGTTCGAGTTTGCCCTGGACTATAAG
AACTTCTCTCGCACAGACGCCGATTACATCAAGAAGTGGAAGCTGTACTC
CTACGGCAACCGGATCAGAATCTTCCGGAATCCTAAGAAGAACAACGTGT
TCGACTGGGAGGAGGTGTGCCTGACCAGCGCCTATAAGGAGCTGTTCAAC
AAGTACGGCATCAATTATCAGCAGGGCGATATCAGAGCCCTGCTGTGCGA
GCAGTCCGACAAGGCCTTCTACTCTAGCTTTATGGCCCTGATGAGCCTGA
TGCTGCAGATGCGGAACAGCATCACAGGCCGCACCGACGTGGATTTTCTG
ATCAGCCCTGTGAAGAACTCCGACGGCATCTTCTACGATAGCCGGAACTA
TGAGGCCCAGGAGAATGCCATCCTGCCAAAGAACGCCGACGCCAATGGCG
CCTATAACATCGCCAGAAAGGTGCTGTGGGCCATCGGCCAGTTCAAGAAG
GCCGAGGACGAGAAGCTGGATAAGGTGAAGATCGCCATCTCTAACAAGGA
GTGGCTGGAGTACGCCCAGACCAGCGTGAAGCACTAG

As described above, the inventors codon optimized Cas12a sequence to match codon usage of tubulin III (FIG. 7). The codon optimised Cas12a (CodOpt-Cas12a) DNA sequence is represented by SEQ ID NO: 4 wherein altered codons are highlighted in bold:

(SEQ ID NO: 4)
ATG AGT AAG CTG GAG AAG TTC ACC AAC TGC TAC AGC CTG AGC AAG ACC CTG
AGG TTT AAG GCC ATC CCT GTG GGC AAG ACC CAG GAG AAC ATC GAC AAC AAG
CGA CTC CTG GTG GAG GAC GAG AAG AGG GCC GAG GAC TAC AAG GGC GTC AAG
AAG CTG CTT GAC CGC TAC TAC CTG AGT TTC ATC AAC GAC GTG CTC CAT AGC
ATC AAG CTG AAG AAC CTT AAC AAC TAC ATC AGC CTG TTT CGG AAG AAG ACC
CGG ACC GAG AAG GAG AAT AAG GAG CTT GAG AAC CTG GAG ATC AAC CTC CGG
AAG GAG ATC GCC AAG GCC TTC AAG GGC AAC GAG GGC TAC AAG TCC CTG TTC
AAG AAG GAC ATC ATA GAG ACC ATC CTG CCC GAG TTC CTT GAC GAC AAG GAC
GAG ATC GCC CTG GTG AAC AGC TTC AAC GGC TTC ACC ACC GCC TTC ACC GGC
TTC TTC GAC AAC CGG GAG AAC ATG TTT AGC GAG GAG GCC AAG TCT ACC AGC
ATC GCC TTC AGG TGC ATC AAC GAG AAC CTT ACT CGG TAC ATC AGC AAC ATG
GAC ATC TTC GAG AAG GTG GAC GCG ATC TTC GAC AAG CAT GAG GTG CAG GAG
ATC AAG GAG AAG ATC CTC AAC AGC GAC TAC GAC GTC GAG GAC TTC TTC GAG
GGG GAG TTC TTC AAC TTC GTG CTT ACC CAG GAA GGC ATC GAC GTG TAC AAC
GCC ATC ATC GGC GGC TTC GTG ACC GAG TCT GGC GAG AAG ATC AAG GGC CTG
AAC GAG TAC ATC AAT CTC TAC AAT CAG AAG ACC AAA CAG AAG CTT CCC AAG
TTC AAA CCC CTG TAC AAG CAG GTG CTG TCT GAC CGG GAG TCT CTT AGC TTC
TAC GGC GAG GGA TAC ACC TCT GAC GAG GAG GTG CTG GAG GTA TTC CGG AAC
ACC CTG AAT AAG AAC AGT GAG ATC TTC AGC TCT ATC AAG AAA CTG GAG AAG
CTT TTC AAG AAT TTT GAC GAG TAC AGC AGT GCT GGC ATC TTC GTG AAA AAC
GGC CCA GCC ATC AGT ACC ATC TCT AAG GAC ATC TTC GGC GAG TGG AAC GTG
ATC AGG GAC AAG TGG AAC GCC GAG TAC GAC GAC ATC CAC CTT AAG AAG AAG
GCA GTC GTG ACC GAG AAG TAC GAG GAC GAC AGA CGG AAG TCT TTC AAG AAG
ATC GGA AGC TTC AGC TTG GAG CAG CTC CAA GAG TAC GCA GAC GCT GAC CTG
TCC GTG GTG GAG AAG CTG AAG GAG ATT ATT ATC CAG AAG GTG GAC GAG ATT
TAC AAG GTG TAC GGC TCT AGC GAG AAG CTT TTC GAC GCC GAC TTC GTG CTG
GAG AAA TCT CTG AAG AAA AAC GAC GCC GTG GTG GCC ATT ATG AAG GAC CTG
CTG GAC TCT GTG AAG AGC TTC GAG AAC TAC ATC AAG GCC TTC TTC GGC GAA
GGA AAG GAG ACC AAC AGA GAC GAG AGC TTC TAC GGC GAC TTC GTG CTG GCC
TAC GAC ATC CTG CTG AAG GTG GAC CAC ATT TAC GAC GCC ATT AGA AAC TAC
GTG ACC CAG AAG CCT TAC AGC AAG GAC AAA TTC AAG CTT TAC TTC CAG AAC
CCC CAG TTC ATG GGG GGC TGG GAC AAG GAC AAG GAG ACC GAC TAC AGA GCC
ACC ATC CTT AGA TAC GGA TCT AAG TAC TAC CTT GCC ATC ATG GAC AAG AAG
TAC GCC AAG TGC CTG CAG AAG ATT GAC AAG GAC GAC GTG AAC GGA AAC TAC
GAG AAG ATT AAC TAC AAG CTG CTG CCC GGC CCT AAC AAG ATG CTT CCC AAG
GTG TTC TTC AGC AAG AAG TGG ATG GCC TAC TAC AAC CCT AGT GAG GAC ATT
CAG AAG ATC TAC AAG AAT GGC ACC TTC AAG AAG GGC GAC ATG TTC AAC CTT
AAC GAC TGC CAC AAG CTG ATC GAC TTC TTT AAG GAC AGC ATC AGT AGA TAC
CCC AAG TGG TCC AAC GCC TAC GAC TTC AAC TTC TCT GAG ACA GAG AAG TAT
AAG GAC ATT GOT GGT TTT TAC AGG GAG GTG GAG GAG CAG GGC TAC AAG GTG
AGC TTC GAG TCT GCC AGC AAG AAG GAG GTG GAC AAA CTG GTG GAG GAG GGC
AAG CTG TAC ATG TTT CAA ATT TAC AAT AAG GAC TTC AGC GAC AAG AGC CAC
GGC ACT CCT AAT CTG CAC ACC ATG TAC TTC AAA CTG CTT TTC GAC GAG AAC
AAT CAT GGC CAG ATC AGA CTG TCC GGC GGC GCC GAG TTG TTC ATG AGA AGA
GCC AGC CTG AAG AAG GAG GAG CTG GTG GTG CAC CCC GCC AAT TCT CCC ATC
GCT AAC AAG AAC CCC GAC AAC CCC AAG AAG ACT ACC ACC CTT AGC TAC GAC
GTA TAC AAG GAC AAG CGG TTT AGC GAG GAC CAG TAC GAG CTG CAC ATC CCC
ATC GCC ATC AAC AAG TGC CCG AAG AAT ATT TTC AAG ATC AAC ACT GAG GTG
AGA GTC CTG CTG AAG CAC GAC GAC AAC CCC TAC GTG ATC GGC ATC GAC AGA
GGC GAG AGA AAC CTC CTG TAC ATC GTG GTG GTG GAC GGC AAG GGC AAT ATC
GTG GAG CAG TAC AGC CTT AAC GAG ATT ATC AAC AAC TTC AAC GGC ATC AGA
ATT AAG ACC GAC TAC CAC TCC CTG CTG GAC AAG AAG GAA AAG GAG AGA TTC
GAG GCC AGG CAG AAC TGG ACA AGC ATT GAG AAC ATC AAG GAG CTG AAG GCC
GGC TAC ATC AGC CAA GTT GTG CAC AAG ATT TGC GAG CTG GTG GAG AAA TAC
GAC GCC GTG ATC GCC TTG GAG GAC CTC AAC AGC GGC TTC AAG AAC TCT CGG
GTG AAG GTG GAG AAG CAG GTG TAC CAG AAG TTC GAG AAG ATG CTG ATT GAC
AAG CTG AAC TAT ATG GTG GAC AAG AAG AGC AAC CCC TGC GCC ACA GGC GGC
GCT CTG AAG GGC TAC CAA ATC ACC AAC AAG TTC GAG AGC TTC AAG TCA ATG
TCT ACC CAG AAC GGC TTC ATC TTC TAC ATC CCT GCC TGG CTT ACC TCC AAG
ATC GAT CCG AGC ACC GGC TTT GTG AAT TTG CTT AAG ACT AAG TAC ACT TCT
ATC GCC GAC TCC AAA AAG TTC ATT AGC TCT TTC GAC AGA ATC ATG TAT GTG
CCC GAA GAG GAC CTG TTC GAA TTT GCC CTC GAC TAC AAG AAT TTC TCC AGG
ACT GAC GCT GAC TAT ATC AAG AAG TGG AAG CTG TAC AGC TAT GGC AAC AGA
ATC CGA ATC TTC CGC AAC CCA AAG AAG AAC AAT GTC TTC GAT TGG GAG GAG
GTG TGC TTG ACT AGC GCC TAC AAG GAG CTG TTC AAC AAG TAT GGC ATT AAC
TAT CAG CAA GGC GAC ATC CGG GCA CTG CTG TGT GAG CAA TCT GAC AAA GCC
TTT TAC AGC TCT TTT ATG GCT CTT ATG TCT CTC ATG TTG CAG ATG AGA AAC
AGC ATC ACC GGC AGA ACT GAC GTG GAC TTC CTC ATT TCT CCC GTG AAG AAC
TCC GAC GGC ATC TTC TAC GAC TCT AGA AAC TAC GAA GCC CAG GAG AAC GCC
ATC CTG CCC AAA AAC GCC GAC GCC AAC GGC GCC TAC AAC ATC GCC AGA AAG
GTG CTG TGG GCC ATC GGG CAG TTC AAG AAA GCC GAG GAC GAG AAG CTT GAC
AAA GTG AAG ATC GCC ATC AGC AAC AAG GAG TGG CTG GAG TAC GCC CAG ACC
AGC GTG AAG CAC TGA

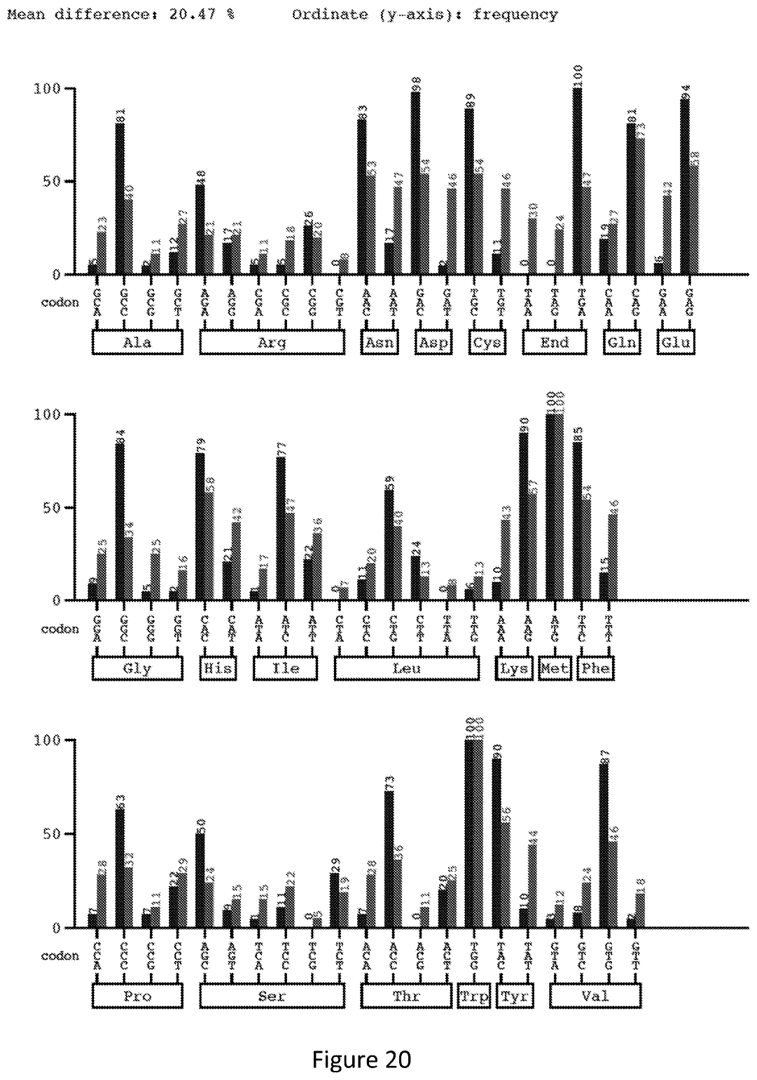

The codon usage frequency of CodOpt-Cas12a (FIG. 20) is similar to that of tubulin III (FIG. 7).

### Codon Optimised Cas13Rx

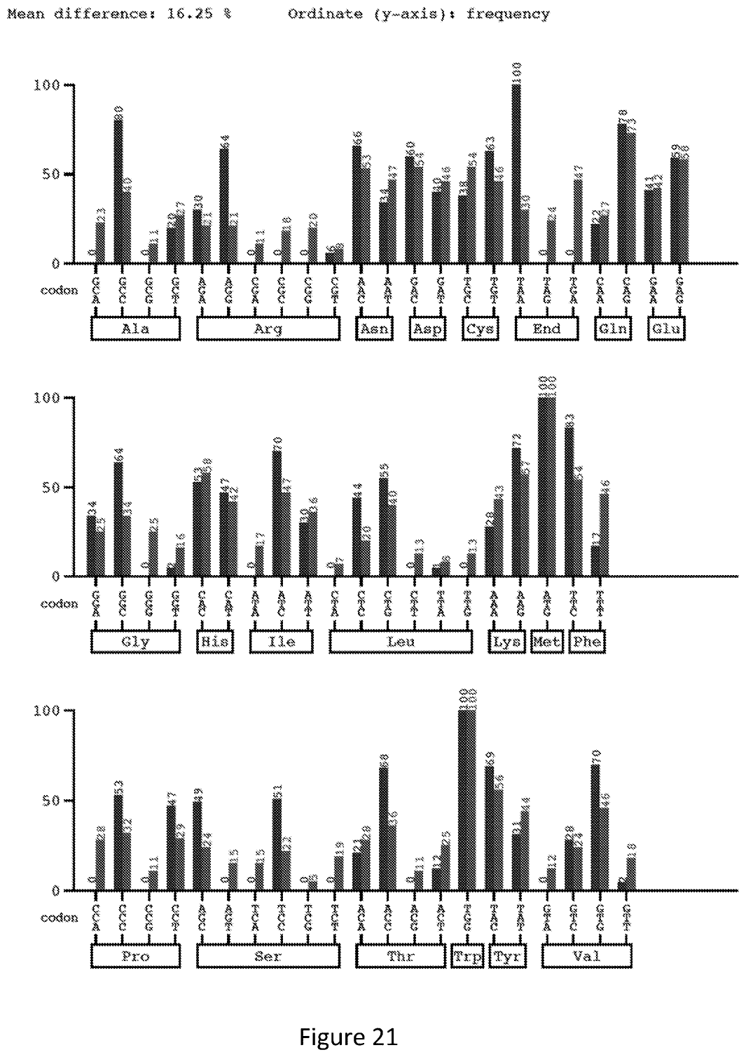

A similar approach was undertaken for Cas13Rx. Codon usage for the existing variant of the Cas13Rx was based on the existing gold standard with optimisation patterns similar to those observed in old-Cas9 (FIG. 21). The starting Cas13Rx sequence was obtained from addgene plasmid ID: 141320 and is represented by SEQ ID NO: 7:

(SEQ ID NO: 7)
ATGAGCGAGGCCAGCATCGAAAAAAAAAAGTCCTTCGCCAAGGGCATGGG
CGTGAAGTCCACACTCGTGTCCGGCTCCAAAGTGTACATGACAACCTTCG
CCGAAGGCAGCGACGCCAGGCTGGAAAAGATCGTGGAGGGCGACAGCATC
AGGAGCGTGAATGAGGGCGAGGCCTTCAGCGCTGAAATGGCCGATAAAAA
CGCCGGCTATAAGATCGGCAACGCCAAATTCAGCCATCCTAAGGGCTACG
CCGTGGTGGCTAACAACCCTCTGTATACAGGACCCGTCCAGCAGGATATG
CTCGGCCTGAAGGAAACTCTGGAAAAGAGGTACTTCGGCGAGAGCGCTGA
TGGCAATGACAATATTTGTATCCAGGTGATCCATAACATCCTGGACATTG
AAAAAATCCTCGCCGAATACATTACCAACGCCGCCTACGCCGTCAACAAT
ATCTCCGGCCTGGATAAGGACATTATTGGATTCGGCAAGTTCTCCACAGT
GTATACCTACGACGAATTCAAAGACCCCGAGCACCATAGGGCCGCTTTCA
ACAATAACGATAAGCTCATCAACGCCATCAAGGCCCAGTATGACGAGTTC
GACAACTTCCTCGATAACCCCAGACTCGGCTATTTCGGCCAGGCCTTTTT
CAGCAAGGAGGGCAGAAATTACATCATCAATTACGGCAACGAATGCTATG
ACATTCTGGCCCTCCTGAGCGGACTGAGGCACTGGGTGGTCCATAACAAC
GAAGAAGAGTCCAGGATCTCCAGGACCTGGCTCTACAACCTCGATAAGAA
CCTCGACAACGAATACATCTCCACCCTCAACTACCTCTACGACAGGATCA
CCAATGAGCTGACCAACTCCTTCTCCAAGAACTCCGCCGCCAACGTGAAC
TATATTGCCGAAACTCTGGGAATCAACCCTGCCGAATTCGCCGAACAATA
TTTCAGATTCAGCATTATGAAAGAGCAGAAAAACCTCGGATTCAATATCA
CCAAGCTCAGGGAAGTGATGCTGGACAGGAAGGATATGTCCGAGATCAGG
AAAAATCATAAGGTGTTCGACTCCATCAGGACCAAGGTCTACACCATGAT
GGACTTTGTGATTTATAGGTATTACATCGAAGAGGATGCCAAGGTGGCTG
CCGCCAATAAGTCCCTCCCCGATAATGAGAAGTCCCTGAGCGAGAAGGAT
ATCTTTGTGATTAACCTGAGGGGCTCCTTCAACGACGACCAGAAGGATGC
CCTCTACTACGATGAAGCTAATAGAATTTGGAGAAAGCTCGAAAATATCA
TGCACAACATCAAGGAATTTAGGGGAAACAAGACAAGAGAGTATAAGAAG
AAGGACGCCCCTAGACTGCCCAGAATCCTGCCCGCTGGCCGTGATGTTTC
CGCCTTCAGCAAACTCATGTATGCCCTGACCATGTTCCTGGATGGCAAGG
AGATCAACGACCTCCTGACCACCCTGATTAATAAATTCGATAACATCCAG
AGCTTCCTGAAGGTGATGCCTCTCATCGGAGTCAACGCTAAGTTCGTGGA
GGAATACGCCTTTTTCAAAGACTCCGCCAAGATCGCCGATGAGCTGAGGC
TGATCAAGTCCTTCGCTAGAATGGGAGAACCTATTGCCGATGCCAGGAGG
GCCATGTATATCGACGCCATCCGTATTTTAGGAACCAACCTGTCCTATGA
TGAGCTCAAGGCCCTCGCCGACACCTTTTCCCTGGACGAGAACGGAAACA
AGCTCAAGAAAGGCAAGCACGGCATGAGAAATTTCATTATTAATAACGTG
ATCAGCAATAAAAGGTTCCACTACCTGATCAGATACGGTGATCCTGCCCA
CCTCCATGAGATCGCCAAAAACGAGGCCGTGGTGAAGTTCGTGCTCGGCA
GGATCGCTGACATCCAGAAAAAACAGGGCCAGAACGGCAAGAACCAGATC
GACAGGTACTACGAAACTTGTATCGGAAAGGATAAGGGCAAGAGCGTGAG
CGAAAAGGTGGACGCTCTCACAAAGATCATCACCGGAATGAACTACGACC
AATTCGACAAGAAAAGGAGCGTCATTGAGGACACCGGCAGGGAAAACGCC
GAGAGGGAGAAGTTTAAAAAGATCATCAGCCTGTACCTCACCGTGATCTA
CCACATCCTCAAGAATATTGTCAATATCAACGCCAGGTACGTCATCGGAT
TCCATTGCGTCGAGCGTGATGCTCAACTGTACAAGGAGAAAGGCTACGAC
ATCAATCTCAAGAAACTGGAAGAGAAGGGATTCAGCTCCGTCACCAAGCT
CTGCGCTGGCATTGATGAAACTGCCCCCGATAAGAGAAAGGACGTGGAAA
AGGAGATGGCTGAAAGAGCCAAGGAGAGCATTGACAGCCTCGAGAGCGCC
AACCCCAAGCTGTATGCCAATTACATCAAATACAGCGACGAGAAGAAAGC
CGAGGAGTTCACCAGGCAGATTAACAGGGAGAAGGCCAAAACCGCCCTGA
ACGCCTACCTGAGGAACACCAAGTGGAATGTGATCATCAGGGAGGACCTC
CTGAGAATTGACAACAAGACATGTACCCTGTTCAGAAACAAGGCCGTCCA
CCTGGAAGTGGCCAGGTATGTCCACGCCTATATCAACGACATTGCCGAGG
TCAATTCCTACTTCCAACTGTACCATTACATCATGCAGAGAATTATCATG
AATGAGAGGTACGAGAAAAGCAGCGGAAAGGTGTCCGAGTACTTCGACGC
TGTGAATGACGAGAAGAAGTACAACGATAGGCTCCTGAAACTGCTGTGTG
TGCCTTTCGGCTACTGTATCCCCAGGTTTAAGAACCTGAGCATCGAGGCC
CTGTTCGATAGGAACGAGGCCGCCAAGTTCGACAAGGAGAAAAAGAAGGT
GTCCGGCAATTCCGGATCCGGATAA

The Cas13Rx sequence was codon optimised using the codon biases of tubulin III (FIG. 7). The DNA sequence of codon optimised Cas13Rx (CodOpt-Cas13Rx) is represented by SEQ ID NO: 5, wherein altered codons are highlighted in bold:

(SEQ ID NO: 5)
ATG AGC GAG GCC AGC ATC GAG AAG AAG AAA TCT TTC GCC AAG GGC ATG GGC
GTG AAG AGC ACC CTG GTG TCT GGC AGC AAG GTG TAC ATG ACC ACC TTC GCC
GAG GGC TCT GAC GCC CGG CTG GAG AAG ATA GTT GAG GGC GAC AGC ATC CGG
AGC GTG AAC GAG GGC GAG GCC TTC TCA GCC GAG ATG GCC GAC AAG AAC GCC
GGC TAC AAG ATT GGG AAC GCG AAG TTT AGT CAT CCC AAG GGC TAC GCC GTG
GTG GCC AAC AAC CCC CTG TAC ACC GGC CCC GTG CAG CAG GAC ATG CTG GGC
CTG AAG GAG ACC CTG GAG AAG AGG TAC TTC GGC GAG TCT GCC GAC GGC AAC
GAC AAC ATC TGC ATC CAG GTG ATC CAC AAC ATC CTG GAC ATC GAG AAG ATC
CTG GCC GAG TAC ATC ACC AAC GCC GCC TAC GCC GTG AAC AAC ATC AGC GGC
CTG GAC AAG GAC ATT ATC GGC TTT GGC AAG TTT TCT ACC GTG TAC ACC TAC
GAC GAG TTC AAA GAC CCT GAA CAT CAT CGG GCC GCC TTC AAC AAC AAC GAT
AAG CTG ATT AAC GCC ATC AAG GCC CAG TAC GAC GAG TTC GAC AAC TTC CTG
GAC AAC CCA CGA CTG GGC TAC TTT GGC CAG GCT TTC TTC AGC AAG GAG GGA
AGA AAC TAC ATC ATC AAC TAC GGA AAC GAG TGC TAT GAC ATT CTC GCC CTC
CTG TCT GGC CTG AGA CAC TGG GTC GTA CAC AAC AAC GAG GAG GAG TCT CGG
ATT AGC AGA ACC TGG CTG TAC AAC CTG GAT AAA AAC CTC GAC AAC GAG TAC
ATC TCT ACC CTT AAC TAC CTG TAC GAC AGA ATC ACC AAC GAG CTC ACC AAT
TCT TTC TCT AAG AAC TCT GCC GCC AAC GTG AAC TAC ATT GCC GAG ACC CTG
GGG ATT AAC CCC GCC GAG TTC GCC GAG CAG TAC TTC AGA TTC AGC ATT ATG
AAG GAG CAG AAG AAC CTG GGC TTT AAC ATC ACC AAG CTG AGA GAG GTG ATG
CTG GAC AGG AAG GAC ATG AGC GAG ATC CGA AAG AAC CAT AAG GTG TTC GAC
AGC ATC AGG ACC AAG GTG TAC ACC ATG ATG GAC TTC GTC ATC TAC AGG TAC
TAC ATC GAG GAG GAC GCC AAG GTG GCT GCG GCA AAC AAG AGC CTG CCT GAT
AAC GAG AAG AGC CTG TCT GAG AAG GAC ATC TTC GTG ATC AAT CTG AGA GGT
TCT TTC AAC GAC GAC CAA AAG GAC GCC CTG TAC TAT GAC GAA GCC AAC AGG
ATT TGG CGA AAG CTG GAG AAC ATC ATG CAC AAC ATC AAG GAG TTC AGG GGC
AAT AAG ACA CGC GAG TAC AAG AAG AAG GAC GCC CCC AGA CTG CCC AGA ATT
CTG CCC GCC GGC AGG GAT GTG AGC GCC TTC TCT AAG CTG ATG TAT GCC CTG
ACC ATG TTT CTG GAC GGC AAA GAG ATC AAC GAC CTG TTG ACC ACC TTG ATC
AAC AAA TTT GAC AAC ATC CAG AGC TTC CTG AAG GTG ATG CCC TTG ATC GGC
GTG AAC GCC AAG TTC GTG GAG GAG TAC GCC TTC TTC AAA GAC TCT GCC AAG
ATT GCC GAC GAA CTG AGA CTG ATC AAG TCT TTC GCC AGG ATG GGA GAG CCC
ATC GCC GAT GCC AGG AGG GCC ATG TAC ATC GAT GCC ATC CGG ATC CTG GGC
ACC AAC CTG TCT TAC GAC GAG CTG AAA GCC CTG GCC GAC ACC TTT TCC CTG
GAC GAG AAC GGC AAC AAG CTT AAG AAG GGC AAG CAC GGC ATG AGA AAC TTC
ATC ATC AAC AAC GTG ATC AGC AAC AAG AGG TTC CAT TAC CTG ATC AGG TAC
GGC GAC CCC GCC CAT CTG CAC GAG ATT GCC AAG AAC GAA GCC GTG GTG AAG
TTC GTG CTG GGC CGG ATT GCT GAC ATC CAG AAG AAG CAA GGC CAG AAC GGC
AAG AAC CAG ATC GAC AGG TAC TAC GAA ACC TGT ATT GGC AAG GAC AAG GGC
AAG AGC GTG TCT GAG AAG GTG GAC GCC CTC ACC AAG ATC ATT ACC GGC ATG
AAC TAC GAC CAG TTC GAC AAG AAG AGG TCT GTG ATT GAA GAC ACC GGA CGG
GAG AAC GCC GAG AGA GAA AAG TTC AAG AAG ATT ATC AGC TTG TAC CTG ACC
GTG ATT TAC CAT ATC CTG AAG AAC ATC GTG AAC ATC AAC GCC CGG TAC GTG
ATC GGC TTC CAC TGC GTG GAG CGG GAC GCC CAG CTG TAC AAG GAG AAG GGC
TAC GAT ATC AAT CTG AAA AAG CTG GAG GAG AAG GGC TTC TCC AGC GTG ACC
AAG CTG TGC GCC GGC ATC GAC GAG ACC GCC CCC GAC AAG CGG AAA GAC GTG
GAG AAG GAG ATG GCC GAG AGG GCC AAG GAG TCT ATC GAC TCT CTG GAG TCT
GCC AAC CCC AAG CTT TAT GCG AAT TAC ATC AAG TAC AGC GAC GAG AAA AAG
GCC GAG GAG TTT ACC AGG CAG ATC AAT CGG GAG AAG GCC AAA ACC GCC CTG
AAC GCC TAC CTG CGC AAC ACC AAG TGG AAC GTG ATT ATC CGG GAG GAC CTG
CTG CGG ATT GAC AAC AAG ACC TGC ACC TTG TTC AGG AAC AAG GCC GTG CAT
CTG GAG GTG GCC AGG TAC GTG CAC GCC TAC ATT AAC GAC ATC GCC GAG GTG
AAT TCC TAC TTT CAG CTG TAC CAC TAC ATA ATG CAA AGA ATC ATC ATG AAC
GAA AGG TAC GAG AAG AGC AGC GGC AAG GTG AGC GAG TAC TTC GAC GCC GTG
AAC GAC GAG AAG AAA TAC AAC GAT CGG CTC CTG AAG CTG CTG TGT GTG CCC
TTT GGC TAC TGC ATC CCT AGA TTC AAG AAC CTT TCT ATC GAG GCC CTG TTC
GAC CGG AAC GAG GCC GCC AAG TTT GAT AAG GAG AAA AAG AAG GTG AGG GGC
AAC AGC GGC AGC GGC TGA

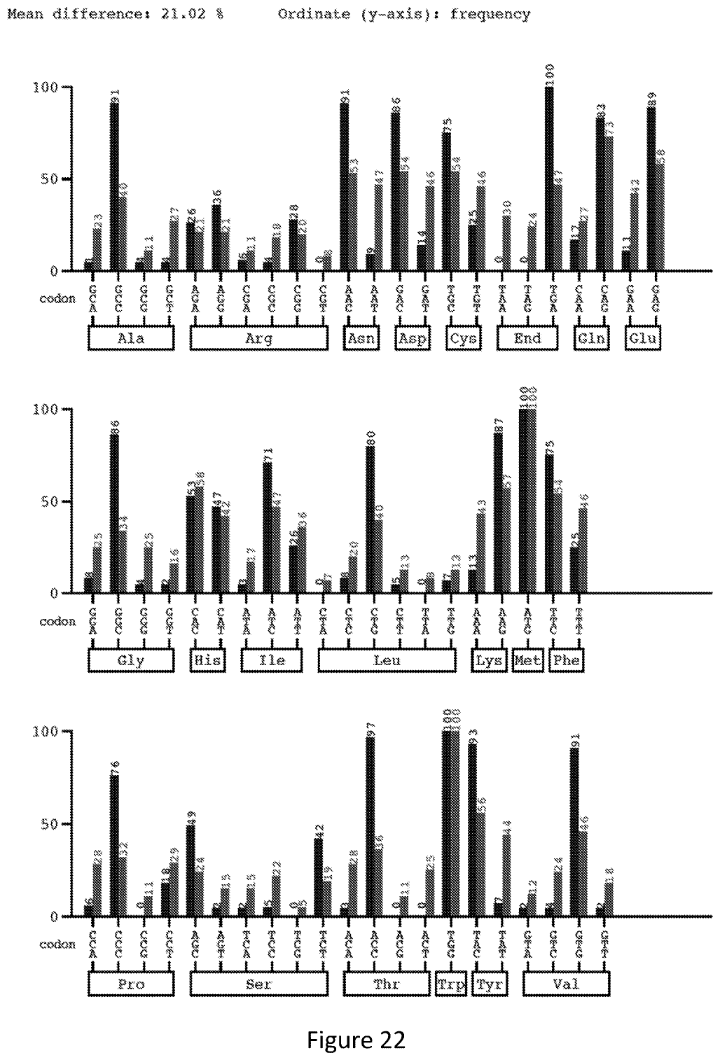

The codon usage frequency of CodOpt-Cas13Rx (FIG. 22) is similar to that of tubulin III (FIG. 7).

Based on the promising results presented herein in relation to Cas9, the inventors postulate that other codon optimised genes, such as the codon optimized variants of Cas12a and Cas13Rx described herein, would be beneficial for carrying out genome editing in various iPSC derived cell types, e.g. neurons and hepatocytes.

### Example 3

### Codon Optimisation of L-Lactate Dehydrogenase

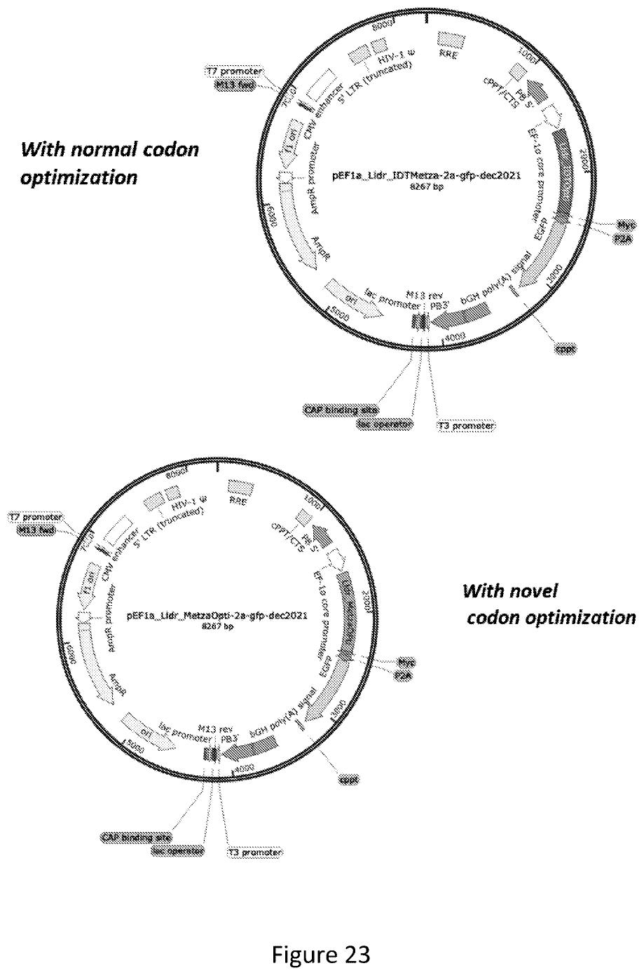

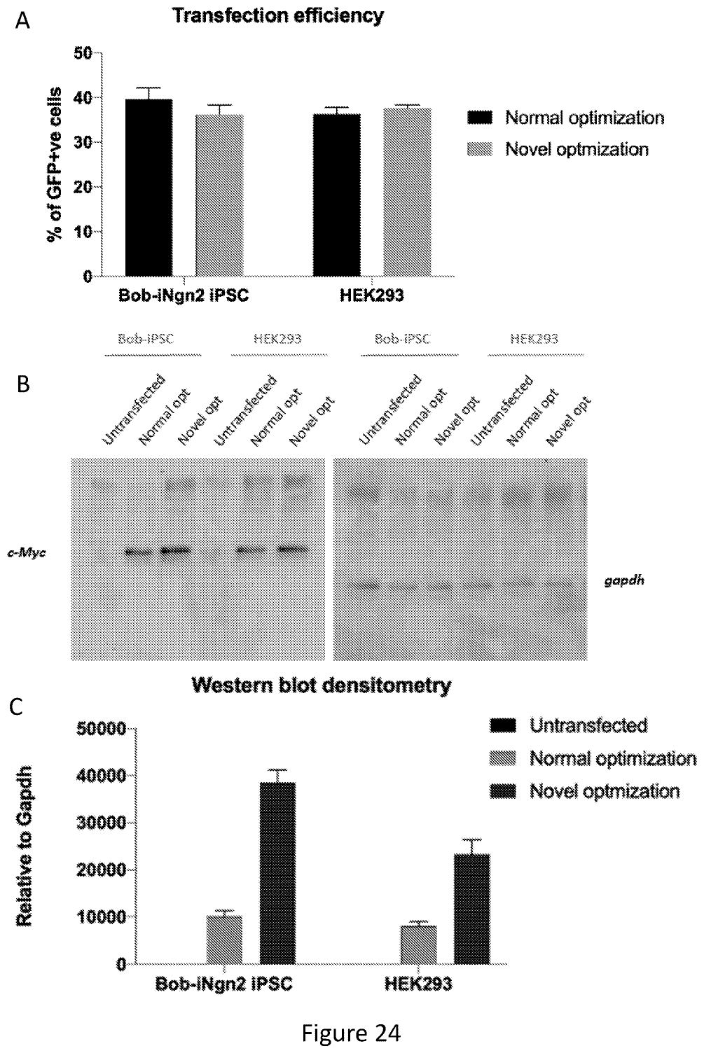

The inventors next sought to confirm that the novel codon optimization technique could be applied to other bacterial derived genes. LIdr from E. coli (which constitutes the L-lactate dehydrogenase operon elements) was codon optimised using the existing gold standard method based on human codon usage frequency (denoted in FIGS. 23 and 24 as “normal optimization”) and using the codon biases of tubulin III as described herein (denoted in FIGS. 23 and 24 as “novel optimization”) and used to construct two plasmids. Both plasmids also harboured eGFP fluorescent reporter to enable assessment of transfection efficiency.

HEK293 cells and iPSCs were transfected with either the plasmid carrying the gold standard (normal) optimised gene or the plasmid carrying the tubulin III (novel) optimised gene. Transfection efficiency was measured 3 days post transfection using flow cytometry (CytoFLEX, Beckman Coulter Life Sciences, Indianapolis US). Cell pellets were collected 5 days post transfection for purposes of Western blotting to determine expression levels of the LIdr gene using the c-myc tagged antibody.

The starting E. coli LIDr sequence is represented by SEQ ID NO: 9:

(SEQ ID NO: 9)
ATGATTGTTTTACCCAGACGCCTGTCAGACGAGGTTGCCGATCGTGTGCG
GGCGCTGATTGATGAAAAAAACCTGGAAGCGGGCATGAAGTTGCCCGCTG
AGCGCCAACTGGCGATGCAACTCGGCGTATCACGTAATTCACTGCGCGAG
GCGCTGGCAAAACTGGTGAGTGAAGGCGTGCTGCTCAGTCGACGCGGCGG
CGGGACGTTTATTCGCTGGCGTCATGACACATGGTCGGAGCAAAACATCG
TCCAGCCGCTAAAAACACTGATGGCCGATGATCCGGATTACAGTTTCGAT
ATTCTGGAAGCCCGCTACGCCATTGAAGCCAGCACCGCATGGCATGCGGC
AATGCGCGCCACACCTGGCGACAAAGAAAAGATTCAGCTTTGCTTTGAAG
CAACGCTAAGTGAAGACCCGGATATCGCCTCACAAGCGGACGTTCGTTTT
CATCTGGCGATTGCCGAAGCCTCACATAACATCGTGCTGCTGCAAACCAT
GCGCGGTTTCTTCGATGTCCTGCAATCCTCAGTGAAGCATAGCCGTCAGC
GGATGTATCTGGTGCCACCGGTTTTTTCACAACTGACCGAACAACATCAG
GCTGTCATTGACGCCATTTTTGCCGGTGATGCTGACGGGGCGCGTAAAGC
AATGATGGCGCACCTTAGTTTTGTTCACACCACCATGAAACGATTCGATG
AAGATCAGGCTCGCCACGCACGGATTACCCGCCTGCCCGGTGAGCATAAT
GAGCATTCGAGGGAGAAAAACGCATGA

The LIDr sequence codon optimised based on human codon usage frequency is represented by SEQ ID NO: 10:

(SEQ ID NO: 10)
ATG ATA GTA TTG CCC CGA CGA CTT AGT GAC GAG GTC
GCA GAT CGA GTC AGA GCC CTT ATT GAT GAG AAA AAC
CTT GAA GCA GGA ATG AAG CTT CCC GCA GAA CGG CAG
CTC GCG ATG CAA CTT GGG GTG TCC CGC AAC TCC TTG
CGC GAA GCA CTC GCG AAA CTG GTG AGC GAA GGT GTG
CTC TTG AGT CGC AGG GGC GGT GGT ACA TTC ATC AGG
TGG AGA CAT GAC ACG TGG TCA GAG CAA AAC ATT GTT
CAA CCT CTC AAA ACT CTC ATG GCA GAT GAT CCT GAC
TAT TCA TTT GAC ATT CTC GAG GCC CGG TAC GCC ATA
GAG GCG AGC ACT GCG TGG CAT GCC GCC ATG CGA GCC
ACG CCG GGC GAT AAG GAG AAG ATA CAA CTC TGC TTC
GAG GCC ACC CTG TCA GAG GAT CCT GAC ATT GCG AGT
CAG GCA GAT GTT CGA TTC CAC CTC GCA ATA GCA GAA
GCC TCT CAC AAC ATC GTC CTG TTG CAG ACT ATG CGC
GGA TTT TTT GAT GTC TTG CAA TCC AGC GTC AAA CAC
TCA CGC CAA AGG ATG TAC TTG GTC CCA CCT GTG TTC
TCC CAA CTG ACT GAG CAG CAC CAA GCT GTA ATC GAC
GCA ATT TTT GCG GGC GAC GCT GAT GGT GCA AGG AAG
GCA ATG ATG GCT CAT CTT AGC TTT GTC CAC ACA ACT
ATG AAG AGA TTT GAT GAA GAC CAA GCA AGG CAT GCG
AGA ATA ACA AGG CTG CCT GGA GAA CAC AAT GAA CAC
AGT AGA GAA AAA AAT GCT TGA

The LIDr sequence codon optimised using the codon biases of tubulin III is represented by SEQ ID NO: 11, wherein altered codons are highlighted in bold:

(SEQ ID NO: 11)
ATG ATC GTG CTC CCC AGA AGG CTG TCC GAC GAG GTG
GCC GAC AGA GTC AGA GCC CTG ATC GAC GAG AAG AAC
CTG GAG GCC GGC ATG AAG CTG CCC GCC GAG CGA CAG
CTG GCC ATG CAG CTG GGC GTG AGC AGA AAC AGC CTG
CGC GAG GCC CTG GCC AAG CTC GTG TCT GAG GGC GTC
CTG CTG TCT AGA AGA GGA GGC GGA ACC TTC ATC CGC
TGG AGA CAC GAC ACC TGG AGC GAG CAA AAT ATC GTG
CAG CCT CTG AAG ACC CTG ATG GCG GAC GAC CCC GAC
TAT AGC TTC GAC ATA CTG GAG GCC AGG TAC GCC ATT
GAA GCA TCC ACC GCG TGG CAC GCC GCT ATG AGG GCC
ACC CCC GGA GAC AAG GAG AAG ATC CAG CTG TGC TTC
GAG GCC ACT CTG AGC GAG GAC CCT GAC ATT GCC AGC
CAG GCC GAC GTG AGG TTC CAC CTG GCC ATC GCT GAG
GCC AGC CAC AAC ATC GTG CTG CTG CAG ACC ATG AGA
GGC TTC TTC GAC GTC CTG CAG AGC AGC GTG AAG CAC
TCA AGA CAG AGA ATG TAC CTC GTC CCC CCT GTG TTC
TCC CAG TTG ACA GAG CAG CAC CAG GCC GTG ATA GAC
GCT ATC TTT GCC GGA GAT GCC GAC GGC GCC AGA AAG
GCC ATG ATG GCC CAC CTG AGC TTC GTG CAT ACC ACC
ATG AAG CGC TTC GAC GAG GAC CAG GCT AGA CAC GCC
AGA ATC ACC AGA CTG CCC GGC GAG CAC AAC GAG CAC
TCC AGA GAG AAG AAC GCC TGA

### Results

No significant differences were identified in the transfection efficiencies of iPSC and HEK293 cells by the two plasmids (FIG. 24A). Western blotting demonstrated that the novel optimization approach based on the codon bias of tubulin III resulted in increased expression of LIDr as compared to the normal gold standard optimisation approach in both iPSC and HEK293 cells (FIGS. 24B and 24C).

LIdr gene expression was robustly increased through the tubulin III codon bias based (novel) method of codon optimization in both HEK293 cells and iPSC. These experiments demonstrate that this novel method of codon optimization is beneficial in boosting and protecting target gene expression in iPSC derived cell types and that it is ideally suited to regulating gene expression in target cell types.

These results demonstrate that the codon optimization approach described herein circumvents gene silencing through iPSC differentiation and also boosts transcription and translation of target genes in desired cell types.

### Materials and Methods

### Constructs

All constructs were designed on the backbone generated by Metzakopian et al. Sci Rep. 2017 22; 7(1):2244. These constructs harbour both PiggyBac inverted terminal repeats to enable transposase-mediated genomic integration (PB transposon) and HIV-1 long terminal repeats to allow lentiviral genomic integration (pKLV-PB-backbone). Any novel construct generated, was done so using pre-synthesized geneblocks (IDT) that were integrated into the backbone using Gibson Assembly. The three Cas9 variants used are driven by EF1A promoter and harboured Blasticidine antibiotic resistance. Genomic-loci targeting constructs were generated using Gibson Assembly through PCR fragments amplified from existing plasmids/extracted genomic DNA. Schematics of each construct generated and used in this work are provided in the figures. When stable integration by transposition of the transgene was required, a plasmid encoding PiggyBac transposase (HyPBase (Yusa et al. PNAS 2011 108(4): 1531-1536)) was co-transfected.

### Cell Culture

All materials and plasticware for routine cell culture purposes were obtained from Sigma unless mentioned otherwise.

### HEK293 Cells

HEK293 cells were routinely cultured in Dulbecco's Modified Essential Media (Gibco) supplemented with penicillin (100 U/ml), streptomycin (100 μg/ml), L-glutamine (2 mM) and 15% Fetal Bovine Serum. Cells were split regularly when 70% confluence was reached using Trypsin-EDTA solution (Sigma) and seeding back 1-10th of the population into a new dish.

Bob-iNgn2-opti-ox IPS cells

TRE-inducible Ngn2 driven Bob iPS cells were a kind gift from Dr. Mark Kotter. Bob-iNgn2-iPSCs were cultured and maintained as per established protocols (Pawlowski et al. Stem Cell Reports 2017 8(4):803-812). In brief, iPS cells were maintained, on vitronectin-coated plates, in TeSR E8 complete media on with supplement (Stem Cell). Upon reaching 70% confluence, iPSCs were allowed to detach using 0.5 mM EDTA solution in PBS. After incubation for 5 mins, cells were triturated and seeded back (¼^{th} to ⅙^{th}). When gene-targeting/transfections was required to be performed, cells were brought into single cell suspension using Accutase (Stem Cell) for 5 mins. Suspended cells were spun down, counted and seeded back as per required numbers in E8 media (with Rock inhibitor) on vitronectin coated plates.

### Bob-iNgn2-Opti-Ox Differentiation to Cortical Neurons

To induce cortical neuron differentiation iPSCs were brought to single cell suspension and seeded at a density of 25k cells/cm² on geltrex coated plates. The following day, cells received differentiation media comprising DMEM/F12 (Gibco), N2 supplement (1×), L-glutamine (1×), non-essential amino acids (1×), 2-Mercaptoethanol (5 uM), Pen-Strep (1×) and Doxycycline (1 μg/ml) for 2 consecutive days. From Day 3, cells received differentiation media comprising-Neurobasal (Gibco), B27 supplement (1×), L-glutamine (1×), 2-Mercaptoethanol (5 uM), Pen-Strep (1×), Doxycycline (1 μg/ml), NT3 (4 μg/ml) and BDNF (100 μg/ml). Media was changed every day until day 6 of differentiation and thereafter every other day until the end of experiment.

### Lentivirus Production

Lentivirus was produced in the HEK293 FT cell line, either using the ViraPower Lentiviral Expression System (Invitrogen) according to manufacturer's instruction, or using the lentivirus packaging plasmid psPAX2 (Addgene, Plasmid #12260) and the pMD2.G envelope plasmid containing VSV-G (Addgene, Plasmid #12259) as described in (Dull et. al. J Virol 1998, Cribbs et. al. BMC Biotechnol 2013). HEK293 FT cells were cultured in DMEM supplemented with 10% FBS (Gibco) and grown on 0.02% gelatin (Sigma) coated plates. Viral production was performed in Opti-Mem (Gibco) using established protocols. Virus from the media was harvested 3 days post transfection. The supernatant was passed through a 45 uM PVDF filter and the virus was thereafter pelleted by spinning at 6000 g for 18 hrs at 4° C. The next day virus pellets were dissolved in PBS, aliquoted and stored at −80° C.

### Plasmid Transfections

HEK293 cells and Bob-iNgn2-iPS cells were grown to 70% confluence in 6-well plates. Cells were dissociated with either Trypsin/EDTA or Accutase respectively and re-suspended in media for reverse transfections (approx. 1×10⁶ cells in 250 ul per transfection). All cells were transfected with 200 ng PiggyBac transposase together with 1000 ng of Cas9 construct. Transfections were performed, using Lipofectamine LTX (Invitrogen) for HEK293 cells or Lipofectamine-STEM (Invitrogen) for Bob-iNgn2-iPSC, according to manufacturer's instructions. Media was replaced after 24 h. Stably-transfected cell lines were generated by selection with Blasticidine (10 μg/ml) for at least 10 days post-transfection. Where gRNA plasmids or reporter plasmids were to be transfected, selection was omitted on these.

### Lentivirus Transductions

All transductions were performed on single cell suspended cells at 37° C. in media containing the lentivirus and polybrene (4 μg/ml) (Sigma). Cells were incubated overnight at 37° C. and media was replaced the next day.

### Flow Cytometry Analysis

All cells including non-transfected controls were harvested at regular time intervals-mainly day 4 and day 7 post transfection, and were analysed for BFP/GFP fluorescence in a flow cytometer (CytoFLEX, Beckman Coulter Life Sciences, Indianapolis US).

### Codon Optimization

Codon optimization of Cas9 was performed to reflect codon usage of that of a neuronal pan marker—Tubulin III. Codon usage analysis was carried out for Cas9, Cas12a and Cas13Rx using tools available at https://www.biologicscorp.com/tools/CodonUsageCalculator/

Codons of the target nucleic acid sequence (Cas9/Cas12a/Cas13Rx) were manually scrutinized and changed to codons that were preferred by the reference nucleic acid sequence (Tubulin III) if necessary. Codons were preferentially changed to the highly preferred codon for each amino acid. When multiple codons were to be changed within a sequence of 60 bases, a distribution reflecting codons in the reference sequence was attempted to be achieved. A distribution of nucleotides A, T, G and C was also considered for every 300 bases as sequences having a GC-content of >60% can be difficult to synthesise. Therefore codons rich in A and T were introduced, when necessary and when applicable, for amino acid coded by 3 or more synonymous codons.

### Western Blotting

Cell lysates from either HEK293 cells or Bob-iNgn2-iPSC and neurons were collected post PBS wash during various time points of the experiment. Whole cell protein was extracted using RIPA buffer (SIGMA) supplemented with 1×PIC. Protein amounts were determined using a Bradford assay and 30 μg of lysates were subjected to electrophoresis on 4-15% Mini-PROTEAN® TGX™ Precast Protein Gels (Biorad). Proteins were transferred onto PVDF membranes (Millipore) using Turboblot system (Biorad). Transferred proteins were then immunoblotted for Cas9 ((7A9-3A3) Mouse mAb #14697, dilution 1:800) and Gapdh (Sigma, #G8795, dilution 1:4000).

### Quantitative RT-PCR (RT-qPCR)

Total RNA was extracted using the RNeasy Mini Kit (Qiagen) according to manufacturer's instructions. First strand cDNA was synthesized using qScript cDNA Supermix (Quantabio) according to manufacturer's protocol. All qPCR studies were performed using Sybr green primers designed to amplify CDS of the gene of interest. qPCR runs were performed on QuantStudio Real-Time PCR System (Applied Biosystems). Samples were run in triplicate, from 3 independent experiments, for both gene of interest and house-keeping genes (18S RNA). Expression levels were normalized to 18s RNA.

### Graphical Representation

All graphical representations were generated using the GraphPad Prism 7 software.

SEQ ID NO: 8-Ensembl Transcript TUBB3-208 ENST00000555576.5:

(SEQ ID NO: 8)
ATGAGGGAGATCGTGCACATCCAGGCCGGCCAGTGCGGCAACCAGATCGG
GGCCAAGTTCTGGGAAGTCATCAGTGATGAGCATGGCATCGACCCCAGCG
GCAACTACGTGGGCGACTCGGACTTGCAGCTGGAGCGGATCAGCGTCTAC
TACAACGAGGCCTCTTCTCACAAGTACGTGCCTCGAGCCATTCTGGTGGA
CCTGGAACCCGGAACCATGGACAGTGTCCGCTCAGGGGCCTTTGGACATC
TCTTCAGGCCTGACAATTTCATCTTTGGTCCACATCTGCTTTGA

1. A method for codon optimising a target nucleic acid sequence for expression in a host cell comprising altering the codon usage frequency of the target nucleic acid sequence based on the codon usage frequency of a gene encoding a protein that is highly expressed in the host cell or the codon usage frequency of a gene encoding a protein that is highly expressed in a cell from the same species as the host cell.

2. The method of claim 1, wherein the method comprises substituting one or more non-preferred codons within the target nucleic acid sequence with preferred synonymous codons, wherein:
(a) non-preferred codons are codons used with low frequency by the gene encoding the highly expressed protein; and
(b) preferred codons are codons used with high frequency by the gene encoding the highly expressed protein.

3. The method according to claim 2, wherein non-preferred codons are codons used with lower frequency by the gene encoding the highly expressed protein than would be expected if each synonymous codon was used at random.

4. The method according to claim 2 or claim 3, wherein non-preferred codons are used by the gene encoding the highly expressed protein with a frequency of less than 50%, less than 45%, less than 40%, less than 35%, less than 33%, less than 30%, less than 25%, less than 20%, less than 16%, less than 15%, less than 10%, less than 5%, or 0%.

5. The method according to any one of claims 2-4, wherein preferred codons are codons used with higher frequency by the gene encoding the highly expressed protein than would be expected if each synonymous codon was used at random.

6. The method according to any one of claims 2-5, wherein preferred codons are used by the gene encoding the highly expressed protein with a frequency of at least 50%, at least 55%, at least 60%, at least 65%, at least 70%, at least 75%, at least 80%, at least 85%, at least 90%, at least 95%, or 100%.

7. The method according to any one of claims 2-6, wherein the method comprises replacing at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of non-preferred codons within the target nucleic acid with preferred synonymous codons.

8. The method according to any one of claims 2-7, wherein the method comprises replacing all non-preferred codons within the target nucleic acid that are used with a frequency of 0% by the gene encoding the highly expressed protein with a preferred synonymous codon.

9. The method according to any one of claims 2-8, wherein the method comprises replacing all non-preferred codons with a preferred synonymous codon in a region of the target nucleic acid that encodes the N-terminal region of a protein.

10. The method according to claim 9, wherein the method comprises replacing all non-preferred codons with a preferred synonymous codon in the 5′ region of the target nucleic acid, optionally the first at least 100, at least 200, at least 300, at least 400, at least 500, at least 600, at least 700, at least 800, at least 900, or at least 1000 codons starting from the 5′ end of the target nucleic acid.

11. The method according to any preceding claim, wherein the protein that is highly expressed is a housekeeping protein or a cell marker protein.

12. The method according to claim 11, wherein the protein that is highly expressed is selected from GAPDH, β-tubulin, β-actin, and tubulin III.

13. The method according to claim 12, wherein the protein that is highly expressed is tubulin III.

14. The method according to claim 13, wherein the one or more non-preferred codons are selected from: alanine codons GCA, GCG and GCT; arginine codons AGA and CGT; cysteine codon TGT; glutamine codon CAA; isoleucine codon ATA; leucine codons CTA and TTA; lysine codon AAA; proline codon CCG; serine codon TCC; threonine codons ACA, ACG and ACT; tyrosine codon TAT; valine codons GTA and GTT; and stop codons TAA and TAG.

15. The method according to claim 13 or claim 14, wherein the one or more non-preferred codons are selected from: asparagine codon AAT; aspartic acid codon GAT; glutamic acid codon GAA; glycine codons GGA, GGG and GGT; histidine codon CAC; isoleucine codon ATT; leucine codons CTC, CTT and TTG; phenylalanine codon TTT; proline codon CCA; serine codons TCA and TCG; and valine codon GTC.

16. The method according to any one of claims 13-15, wherein preferred codons are selected from: alanine codon GCC; cysteine codon TGC; glutamine codon CAG; lysine codon AAG; threonine codon ACC; tyrosine codon TAC; and the stop codon TGA.

17. The method according to any one of claims 13-16, wherein preferred codons are selected from: arginine codons AGG, CGA, CGC and CGG; asparagine codon AAC; aspartic acid codon GAC; glutamic acid codon GAG; glycine codon GGC; histidine codon CAT; isoleucine codon ATC; leucine codon CTG; phenylalanine codon TTC; proline codons CCC and CCT; serine codons AGC, AGT and TCT; and valine codon GTG.

18. The method according to any preceding claim, wherein the host cell is selected from a human cell, a bacterial cell, a yeast cell and a fungal cell.

19. The method according to claim 18, wherein the host cell is a human cell.

20. The method according to claim 19, wherein the host cell is a HEK293 cell.

21. The method according to claim 19, wherein the host cell is a human induced pluripotent stem cell (iPSC).

22. The method according to any one of claims 19-21, wherein the host cell is a differentiated cell derived from an iPSC, optionally wherein the host cell is selected from an iPSC derived neuron such as a cortical neuron, dopaminergic neuron or a motor neuron, an iPSC derived macrophage, an iPSC derived cardiomyocytes, and an iPSC derived hepatocyte.

23. The method according to any preceding claim, wherein the target nucleic acid encodes a Cas protein, optionally wherein the Cas protein is selected from Cas9, Cas12a and Cas13Rx.

24. A nucleic acid comprising a nucleic acid sequence that has been codon optimised by the method of any one of claims 1-23.

25. A codon optimised nucleic acid for improved expression in a host cell wherein the codon usage frequency of the nucleic acid corresponds to the codon usage frequency of a gene encoding a protein that is highly expressed by the host cell or the codon usage frequency of a gene encoding a protein that is highly expressed in a cell from the same species as the host cell.

26. The codon optimised nucleic acid according to claim 25, wherein the codon optimised nucleic acid comprises a lower frequency of non-preferred codons than a non-optimised nucleic acid sequence encoding the same amino acid sequence.

27. The codon optimised nucleic acid according to claim 25 or claim 26, wherein the codon optimised nucleic acid comprises a higher frequency of preferred codons than a non-optimised nucleic acid sequence encoding the same amino acid sequence.

28. A nucleic acid encoding Cas9 and comprising a nucleic acid sequence having at least 70%, at least 75%, at least 80%, at least 85%, at least 90%, at least 95% or 100% sequence identity to SEQ ID NO: 1 or SEQ ID NO: 3.

29. A nucleic acid encoding Cas12a and comprising a nucleic acid sequence having at least 70%, at least 75%, at least 80%, at least 85%, at least 90%, at least 95% or 100% sequence identity to SEQ ID NO: 4.

30. A nucleic acid encoding Cas13Rx and comprising a nucleic acid sequence having at least 70%, at least 75%, at least 80%, at least 85%, at least 90%, at least 95% or 100% sequence identity to SEQ ID NO: 5.

31. A vector comprising a nucleic acid according to any one of claims 24-30.

32. A host cell comprising a nucleic acid according to any one of claims 24-30 or a vector according to claim 31.

33. The codon optimised nucleic acid sequence according to any one of claims 24-27 or the host cell comprising a nucleic acid according to claim 32, wherein the host cell is selected from a human cell, a bacterial cell, a yeast cell and a fungal cell.

34. The method according to claim 33, wherein the host cell is a human cell.

35. The method according to claim 34, wherein the host cell is a HEK293 cell.

36. The method according to claim 34, wherein the host cell is a human induced pluripotent stem cell (iPSC).

37. The method according to any one of claims 33-36, wherein the host cell is a differentiated cell derived from an iPSC, optionally wherein the host cell is selected from an iPSC derived neuron such as a cortical neuron, dopaminergic neuron or a motor neuron, an iPSC derived macrophage, an iPSC derived cardiomyocytes, and an iPSC derived hepatocyte.
